(19) United States
(12) Patent Application Publication
(10) Pub. No.: US 2009/0191599 A1
(43) Pub. Date: Jul. 30, 2009

(54) ENGINEERED LIGHT-HARVESTING ORGANISMS

(75) Inventors: Eric James Devroe, Malden, MA (US); David Arthur Berry, Brookline, MA (US); Noubar Boghos Afeyan, Lexington, MA (US); Dan Eric Robertson, Belmont, MA (US); Frank Anthony Skraly, Watertown, MA (US); Christian Perry Ridley, Acton, MA (US)

Correspondence Address: FENWICK & WEST LLP, SILICON VALLEY CENTER, 801 CALIFORNIA STREET, MOUNTAIN VIEW, CA, 94041, US

(73) Assignee: Joule Biotechnologies, Inc., Cambridge, MA (US)

(21) Appl. No.: 12/208,300

(22) Filed: Sep. 10, 2008

Related U.S. Application Data
(60) Provisional application No. 60/971,224, filed on Sep. 10, 2007.
(60) Provisional application No. 61/076,083, filed on Jun. 26, 2008.
(60) Provisional application No. 61/076,096, filed on Jun. 26, 2008.
(60) Provisional application No. 61/079,679, filed on Jul. 10, 2008.
(60) Provisional application No. 61/079,683, filed on Jul. 10, 2008.

(51) Int. Cl. C12P 19/04; C12N 1/21; C12P 5/00

(52) U.S. Cl. 435/101; 435/252.33; 435/166

(57) ABSTRACT

The present disclosure identifies pathways and mechanisms to confer photoautotrophic properties to a heterotrophic organism. The resultant engineered cell or organism will uniquely enable efficient conversion of carbon dioxide and light into biomass and carbon-based products of interest.

### CROSS REFERENCE TO RELATED APPLICATIONS

This application claims priority from U.S. Provisional Applications 60/971,224, filed on Sep. 10, 2007; 61/076,083 filed on Jun. 26, 2008; 61/076,096, filed on Jun. 26, 2008; 61/079,679, filed Jul. 10, 2008; and 61/079,683 filed Jul. 10, 2008, the disclosure of each of which is incorporated by reference herein for all purposes.

### REFERENCE TO SEQUENCE LISTING

This application is filed with an electronically submitted Sequence Listing, herein incorporated by reference in its entirety.

### FIELD

The present disclosure relates to identification of pathways and mechanisms to confer photoautotrophic properties to a heterotrophic organism and in particular to engineering the resultant synthetophototrophic organism to uniquely enable efficient conversion of carbon dioxide and light into biomass and carbon-based products of interest.

### BACKGROUND

Photosynthesis is a process by which biological entities utilize sunlight and CO₂ to produce sugars for energy. Photosynthesis, as naturally evolved, is an extremely complex system with numerous and poorly understood feedback loops, control mechanisms, and process inefficiencies. This complicated system presents likely insurmountable obstacles to either one-factor-at-a-time or global optimization approaches [Nedbal L, Cerven Ã J, Rascher U, Schmidt H. E-photosynthesis: a comprehensive modeling approach to understand chlorophyll fluorescence transients and other complex dynamic features of photosynthesis in fluctuating light. Photosynth Res. 2007 July; 93(1-3):223-34; Salvucci M E, Crafts-Brandner S J. Inhibition of photosynthesis by heat stress: the activation state of Rubisco as a limiting factor in photosynthesis. Physiol Plant. 2004 February; 120(2):179-186; Greene D N, Whitney S M, Matsumura I. Artificially evolved Synechococcus PCC6301 Rubisco variants exhibit improvements in folding and catalytic efficiency. Biochem J. 2007 Jun. 15; 404(3):517-24].

Existing photoautotrophic organisms (i.e., plants, algae, and photosynthetic bacteria) are poorly suited for industrial bioprocessing. In particular, said organisms have a slow doubling time (3-72 hrs) compared to industrialized heterotrophic organisms such as Escherichia coli (20 minutes). In addition, techniques for genetic manipulation (knockout, over-expression of transgenes via integration or episomic plasmid propagation) are inefficient, time-consuming, laborious, or non-existent.

### SUMMARY

Given these shortcomings, the present disclosure identifies pathways and mechanisms to confer photoautotrophic properties to a heterotrophic organism. The resultant engineered synthetophototrophic cell or organism will uniquely enable efficient conversion of carbon dioxide and light into biomass and carbon-based products of interest. In certain aspects, the present invention provides an engineered cell comprising at least two engineered nucleic acids, wherein at least one engineered nucleic acid is selected from a group consisting of a light capture nucleic acid, a carbon dioxide fixation pathway nucleic acid, a NADH pathway nucleic acid, and a NADPH pathway nucleic acid; and wherein a second engineered nucleic acid is selected from a distinct member of said group (i.e., if a first nucleic acid is a light capture nucleic acid, then at least one other nucleic acid must be a carbon dioxide fixation pathway nucleic acid, a NADH pathway nucleic acid, or a NADPH pathway nucleic acid). In a related embodiment, the cell is light dependent or fixes carbon. In yet another related embodiment, the cell has engineered phototrophic activity. In still another related embodiment, said cell is synthetophototrophic or fixed carbon or both. In yet another related embodiment, the cell is photoautotrophic in the presence of light and heterotrophic in the absence of light. In certain related embodiments, at least one engineered nucleic acid in the cell encodes proteorhodopsin. The invention also provides, in related embodiments, an engineered cell where the cell is a microorganism selected from the group consisting of Acetobacter aceti, Bacillus subtilis, Clostridium ljungdahlii, Clostridium thermocellum, Escherichia coli, Penicillium chrysogenum, Pichia pastoris, Saccharomyces cerevisiae, Schizosaccharomyces pombe, Pseudomonas fluorescens and Zymomonas mobilis.

In related embodiment, at least one of the engineered nucleic acids in the engineered cell is an exogenous nucleic acid. In other embodiments, at least one of the engineered nucleic acids is a modified endogenous gene. In certain aspects, the present invention provides an engineered cell comprising at least three engineered nucleic acids, wherein at least one engineered nucleic acid is selected from a group consisting of a light capture nucleic acid, a carbon dioxide fixation pathway nucleic acid, a NADH pathway nucleic acid, and a NADPH pathway nucleic acid; and wherein a second engineered nucleic acid is selected from a distinct member of said group; and wherein a third engineered nucleic acid is an additional modified endogenous gene, e.g., a gene from one of the above-mentioned four groups. In a related embodiment, said engineered nucleic acids are selected from at least three members of the group consisting of a light capture nucleic acid, a carbon dioxide fixation pathway nucleic acid, a NADH pathway nucleic acid, and a NADPH pathway nucleic acid. In yet another related embodiment, the cell of the invention comprises at least one engineered light capture nucleic acid, at least one engineered carbon dioxide fixation pathway nucleic acid, at least one engineered NADH pathway nucleic acid, and at least one engineered NADPH pathway nucleic acid. In yet another embodiment, the engineered cell of the invention comprises at least one engineered light capture nucleic acid and at least one engineered carbon dioxide fixation pathway nucleic acid.

In related embodiments of the engineered cell of the invention, at least one engineered nucleic acid is a light capture nucleic acid selected from the group consisting of proteorhodopsin, bacteriorhodopsin, deltarhodopsin, xanthorhodopsin, Leptosphaeria maculans opsin, isopentenyl-diphosphate delta-isomerase, 15,15′-beta-carotene dioxygenase, lycopene cyclase, phytoene synthase, phytoene dehydrogenase, geranylgeranyl pyrophosphate synthetase, beta-carotene ketolase, photosystem P840 reaction center large subunit, pscA, photosystem P840 reaction center iron-sulfur protein, pscB, photosystem P840 reaction center cytochrome c-551, pscC, photosystem P840 reaction center protein, pscD, bacteriochlorophyl a binding protein, Fenna-Mathews-Olson protein, FMO, Photosystem I P700 chlorophyll A apoproptein A1, psaA, Photosystem I P700 chlorophyll A apoproptein A2, psaB, Photosystem I iron-sulfur center subunit VII, psaC, Photosystem I reaction center subunit II, psaD, Photosystem I reaction centre subunit IV PsaE, Photosystem I reaction centre subunit IX PsaJ, Photosystem I reaction centre subunit III precursor (PSI-F), Photosystem I reaction centre subunit XII PsaM, Photosystem I reaction center subunit PsaK, Photosystem I assembly protein, Photosystem I subunit VIII PsaI, Photosystem I reaction centre subunit XI PsaL, Photosystem II protein X PsbX, Photosystem II reaction center D1, Photosystem II manganese-stabilizing protein PsbO, Photosystem II 10 kDa phosphoprotein PsbH, Photosystem II reaction center N protein PsbN, Photosystem II protein PsbI, Photosystem II protein PsbK, Photosystem II stability/assembly factor, Cytochrome b559 alpha subunit PsbE, Cytochrome b559 beta chain PsbF, Photosystem II protein L PsbL, Photosystem II protein J PsbJ, PucC protein, Photosystem II reaction center T PsbT, Photosystem II chlorophyll a-binding protein CP47 homolog, Photosystem II protein M PsbM, Photosystem II protein Psb27, Photosystem II protein Y PsbY, Photosystem II reaction centre W protein, Photosystem II protein P PsbP, Flavodoxin, IsiB, Photosystem II reaction center D2, Photosystem II chlorophyll a-binding protein CP43 homolog, and a Homolog of PsbF protein. In a related embodiment, the cell generates proton motive force, wherein the proton motive force promotes the growth of said cell in a light-dependent manner. In related embodiments, the growth of the engineered cell is in the presence of salt. In certain embodiments, the proton motive force is generated by proteorhodopsin. In yet other related embodiments, the engineered cell further comprises engineered rbcL nucleic acid, engineered rbcS nucleic acid, and engineered phosphoribulokinase.

In certain embodiments of the engineered cell of the invention, the at least one engineered nucleic acid is a carbon dioxide fixation pathway nucleic acid selected from the group consisting of a functional hydroxyproprionate cycle nucleic acid, a reductive TCA cycle nucleic acid, a reductive acetyl coenzyme A pathway nucleic acid, a reductive pentose phosphate cycle nucleic acid, a glyoxylate shunt pathway nucleic acid, a Calvin cycle nucleic acid and a gluconeogenesis pathway nucleic acid. In related embodiments, the at least one engineered nucleic acid is a carbon dioxide fixation pathway nucleic acid selected from the group consisting of acetyl-CoA carboxylase (subunit alpha), acetyl-CoA carboxylase (subunit beta), biotin-carboxyl carrier protein (accB), biotin-carboxylase, malonyl-CoA reductase, 3-hydroxypropionyl-CoA synthase, propionyl-CoA carboxylase (subunit alpha), propionyl-CoA carboxylase (subunit beta), methylmalonyl-CoA epimerase, methylmalonyl-CoA mutase, succinyl-CoA:L-malate CoA transferase (subunit alpha), succinyl-CoA:L-malate CoA transferase (subunit beta), fumarate reductase—frdA—flavoprotein subunit, fumarate reductase iron-sulfur subunit-frdb, g15 subunit [fumarate reductase subunit c], g13 subunit [fumarate reductase subunit D], fumarate hydratase—class I aerobic (fumA), L-malyl-CoA lyase, ATP-citrate lyase, subunit 1, ATP-citrate lyase, subunit 2, citryl-CoA synthase (large subunit, citryl-CoA synthase (small subunit), citryl-CoA ligase, malate dehydrogenase, fumarase hydratase (aerobic isozyme, fumA), succinate dehydrogenase (flavoprotein subunit—SdhA), SdhB iron-sulfur subunit, SdhC membrane anchor subunit, SdhD membrane anchor subunit, succinyl-CoA synthetase subunit alpha (sucD), succinyl-CoA synthetase subunit beta (sucC), alpha-ketoglutarate subunit alpha-korA, alpha-ketoglutarate subunit beta-korB, isocitrate dehydrogenase—NADP dependent, isocitrate dehydrogenase—NAD dependent Subunit 1, isocitrate dehydrogenase—NAD depend. Subunit 2, aconitate hydratase 1 (acnA), aconitate hydratase 2 (acnB), pyruvate synthase, subunit A porA, pyruvate synthase, subunit B porB, pyruvate synthase, subunit C porC, pyruvate synthase, subunit D porD, phosphoenolpyruvate synthase—ppsA, PEP carboxylase, ppC, NADP-dependent formate dehydrogenase—subunit A Mt-fdhA, NADP-dependent formate dehydrogenase—subunit B Mt-fdhB, formate tetrahydrofolate ligase, methenyltetrahydrofolate cyclohydrolase, methylene tetrahydrofolate reductase, metF, 5-methyltetrahydrofolate corrinoid/iron sulfur protein methyltransferase, acsE, carbon monoxide dehydrogenase/acetyl-CoA synthase—subunit alpha, carbon monoxide dehydrogenase/acetyl-CoA synthase—subunit beta, malate synthase—aceB, isocitrate lyase—aceA, malate dehydrogenase, pyruvate carboxylase, phosphoenolpyruvate carboxykinase, fructose-1,6-bisphosphatase, glucose-6-phosphatase—dog1, pyruvate ferredoxin:oxidoreductase with pyruvate synthase activity, fructose-1,6-bisphosphatase (FBPase) and sedoheptulose-1,7-bisphosphatase (SBPase), bifunctional, cbbF, glyceraldehyde-3-phosphate dehydrogenase (GAPDH), cbbG, phosphoribulokinase (PRK), cbbP, CP12, transketolase, cbbT, fructose 1,6-bisphosphate aldolase, cbbA, pentose-5-phosphate-3-epimerase, cbbE, ribose 5-phosphate isomerase, phosphoglycerate kinase, triosephosphate isomerase, tpiA, Ribulose-1,5-bisphosphate carbyxlase/oxygenase (RubisCo)—small subunit—cbbS, Ribulose-1,5-bisphosphate carbyxlase/oxygenase (RubisCo)—large subunit cbbL, Rubisco activase, rbcL, rbcS, Salinibacter fructose-bisphosphate aldolase, Synechococcus sp. 7002 fructose-bisphosphate aldolase (class I), Synechococcus elongatus PCC 7942 sedoheptulose-1,7-bisphosphatase, and T. elongatus BP-1 sedoheptulose-1,7-bisphosphatase. In other related embodiments, the at least one engineered nucleic acid is a codon-optimized carbon dioxide fixation pathway nucleic acid selected from the group consisting of Salinibacter fructose-bisphosphate aldolase, Synechococcus sp. 7002 fructose-bisphosphate aldolase (class I), Synechococcus elongatus PCC 7942 sedoheptulose-1,7-bisphosphatase, and T. elongatus BP-1 sedoheptulose-1,7-bisphosphatase. In a related embodiment, the cell generates proton motive force, wherein the proton motive force promotes the growth of said cell in a light-dependent manner. In another related embodiment, the growth of the engineered cell is in the presence of salt. In certain embodiments, the proton motive force is generated by proteorhodopsin. In yet other related embodiments, the engineered cell further comprises engineered rbcL nucleic acid, engineered rbcS nucleic acid, and engineered phosphoribulokinase. In yet another related embodiment, the carbon dioxide fixation pathway nucleic acid comprised by the engineered cell is a Woods-Ljungdahl pathway nucleic acid. In still another related embodiment, the cell further comprises an engineered glyoxylate shunt pathway nucleic acid and an exogenous gluconeogenesis pathway nucleic acid.

In another embodiment of the engineered light-capturing cell of the invention, at one least one engineered nucleic acid is a NADH pathway nucleic acid selected from the group consisting of soluble pyridine nucleotide transhydrogenase—udhA, membrane-bound pyridine nucleotide transhydrogenase—pntAB, NAD+-dependent isocitrate dehydrogenase—idh, NAD+-dependent isocitrate dehydrogenase—idh2, malate dehydrogenase, and NADH:ubiquinone oxidoreductase—OPERON (a-n). In a related embodiment, the at least one engineered nucleic acid is an endogenous NADH pathway nucleic acid selected from the group consisting of a nuo gene, a ndh gene, cytochrome bo, and cytochrome bd. In yet another related embodiment, the endogenous NADH pathway nucleic acid comprises a deletion or modification that disrupts said pathway. In another embodiment, the engineered cell of the invention comprises at least two engineered NADH pathway nucleic acids, wherein said at least two engineered NADH pathway nucleic acids include a soluble pyridine nucleotide dehydrogenase and a NAD⁺-dependent isocitrate dehydrogenase.

In another embodiment of the light-capturing cell of the invention, at least one engineered nucleic acid is a NADPH pathway nucleic acid selected from the group consisting of glucose-6-phosphate dehydrogenase, zwf 6-phosphogluconolactonase -pgi, 6-phosphogluconate dehydrogenase, gnd, NADP-dependent isocitrate dehydrogenase, NADP-dependent malic enzyme, soluble pyridine nucleotide transhydrogenase—udhA, or membrane-bound pyridine nucleotide transhydrogenase, subunit alpha, pntA and subunit beta, pntB. In a related embodiment, the engineered cell comprises at least two engineered NADPH pathway nucleic acids, wherein said at least two NADPH pathway nucleic acids include a soluble nucleotide dehydrogenase and a glucose-6-phosphate dehydrogenase. In yet another embodiment, one or more acetyl-CoA flux nucleic acids in the engineered cell are expressed or inhibited.

In other aspects, the present invention provides a host cell, wherein said host cell is engineered to capture light and fix carbon dioxide. In preferred embodiments, the present invention provides a host cell generating proton motive force, wherein said proton motive force promotes light-dependent growth of said cell. In related embodiments, the light-dependent growth of cell is in the presence of salt. The salt concentration in some embodiments is about 0.3 M. In some embodiments, the salt concentration is at least 0.3 M, e.g., between 0.3 M and 0.5 M.

In further aspects, the present invention provides a method for producing biological sugars, hydrocarbon products, solid forms of carbon, fuels, biofuels or pharmaceutical agents comprising culturing an engineered cell in the presence of CO2 and light under conditions sufficient to produce the carbon products and collecting or separating the carbon.

### BRIEF DESCRIPTION OF THE FIGURES

FIG. 1 shows typical inputs and outputs corresponding to an oxygenic photosynthetic organism. The engineered light-harvesting organisms in the present invention utilize the same inputs and intermediates, though oxygen output formation is optional.

FIG. 2 depicts the capture of light via a light-driven proton pump, such as proteorhodopsin. After Walter J M, Greenfield D, Bustamante C, Liphardt J. “Light-powering Escherichia coli with proteorhodopsin.” PNAS (2007). 104(7):2408-2412.

FIG. 3 illustrates absorption spectra of two different proteorhodopsin pumps expressed in E. coli and the spectrum exhibited by human rhodopsins.

FIG. 4 depicts expression of proteorhodopsin in E. coli BL21 DE(3). (A) Duplicate cultures of JCC349 induced with 0.1 mM IPTG in the presence or absence of 20 μM trans-retinal (B) Visible scan of the JCC349 culture incubated with retinal using the retinal-minus strain as the blank.

FIG. 5 represents growth for JCC349 in 0.3 M sodium chloride under green light. (A) Green LED array and aquarium setup (B) Bubble tubes of duplicate culture of JCC349 incubated in M9 media or in M9 media supplemented with 0.3M sodium chloride either under illumination by the green LED array or in the dark (C) Bubble tubes of duplicate culture of JCC349 incubated in M9 media supplemented with 0.3M sodium chloride either under illumination by the green LED array or in the dark (D) Pellets from 5 mls of cultures after resuspension in 1 ml Milli-Q water (1,2=M9 media in light; 3,4=M9/0.3M NaCl in light; 5,6=M9 media in dark; 7,8=M9/0.3M NaCl in dark).

FIG. 6 shows a graphical representation of overnight growth of JCC308-309 and JCC311-312 in M9/0.2% L-arabinose. (A) Growth in culture tubes while induced with IPTG (B) Overnight growth of JCC308 and JCC311 in bubble tubes (bt) and culture tubes (ct) while induced with IPTG.

FIG. 7 shows the results of co-expression of proteorhodopsin with prkA and RUBISCO genes. (A) Duplicate culture of JCC351 induced with 0.1 mM IPTG in the presence or absence of 20 μM trans-retinal (B) Growth of JCC 349 and JCC351-352 in bubble tubes while induced with IPTG (C) Growth of JCC 349 and JCC351-352 in culture tubes with and without 20 μM trans-retinal (D) Growth of JCC351 and JCC352 in bubble tubes (bt) and culture tubes (ct).

FIG. 8 is a schematic representation of glycogen biosynthesis after ¹³C incorporation into 3-phosphoglycerate catalyzed by RUBSICO. “*” indicates ¹³C label. Unshaded arrow indicates non-biosynthetic acid glycogen hydrolysis product glucose. Biosynthetic scheme indicates product if both 3-phosphoglyceraldehyde and dihydroxyacetone-phosphate (DHAP) are labeled. Since both labeled and non-labeled 3-phosphoglyceraldehyde are biosynthesized, four populations of glucose are anticipated as product [C-3, C-4 labeled]: [C-3 labeled]: [C-4 labeled]: [neither labeled] in a 1:1:1:1 ratio.

FIG. 9 shows a pathway for CO₂ assimilation in Crenarchaeota via 3-hydroxypropionate (3-HPA) cycle. After Hallam S J, Mincer T J, Schleper C, Preston C M, Roberts K, Richardson P M, DeLong. Pathways of carbon assimilation and ammonia oxidation suggested by environmental genomic analyses of marine Crenarchaeota. PLoS Biol. 2006 April; 4(4):e95.

FIG. 10 depicts a pathway for CO₂ fixation by Chloroflexus aurantiacus via 3-hydroxypropionate (3-HPA) cycle. After Herter S, Farfsing J, Gad'On N, Rieder C, Eisenreich W, Bacher A, Fuchs G. Autotrophic CO(2) fixation by Chloroflexus aurantiacus: study of glyoxylate formation and assimilation via the 3-hydroxypropionate cycle. J. Bacteriol. 2001 July; 183(14):4305-16.

FIG. 11 depicts a pathway for CO₂ assimilation via reductive acetyl-CoA pathway (Woods-Ljungdahl Pathway).

FIG. 12 depicts a pathway for CO₂ assimilation via reductive tricarboxylic acid (rTCA) cycle.

FIG. 13 depicts a pathway for gluconeogenesis.

FIG. 14 depicts an altered pathway for gluconeogenesis employing pyruvate:ferredoxin oxidoreductase (PFOR) to obtain pyruvate.

FIG. 15 illustrates the generation of inputs for gluconeogenesis using the glyoxylate shunt.

FIG. 16 illustrates the production of NADPH via the pentose phosphate pathway.

FIG. 17 illustrates the production of NADH by Rhodobacter sphaeroides based on denitrification.

FIG. 18 illustrates the generation of ATP and NADPH by Rhodobacter.

FIG. 19 illustrates comparative electron flow in anoxygenic photosynthetic bacteria.

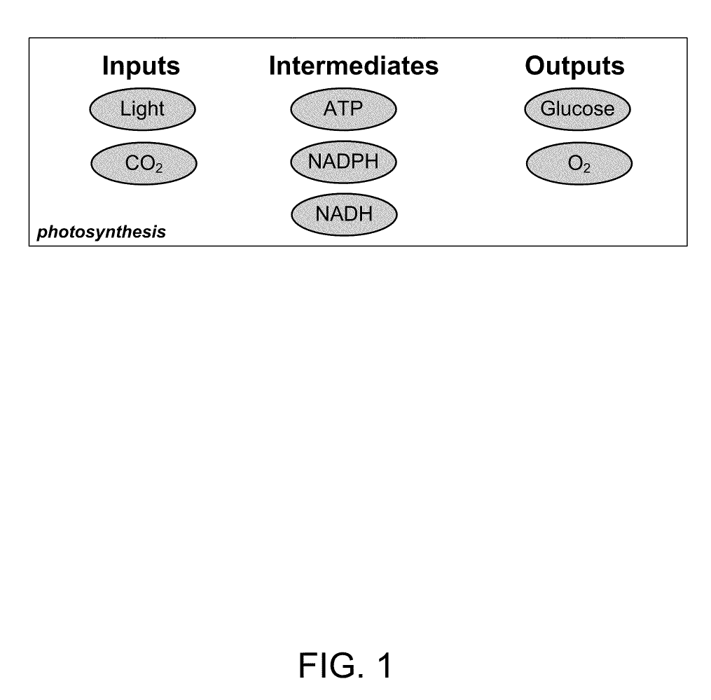

### ABBREVIATIONS AND TERMS

The following explanations of terms and methods are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. For example, reference to “comprising a cell” includes one or a plurality of such cells, and reference to “comprising the thioesterase” includes reference to one or more thioesterase peptides and equivalents thereof known to those of ordinary skill in the art, and so forth. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

Accession Numbers The accession numbers throughout this description are derived from various public databases, including NCBI database (National Center for Biotechnology Information) maintained by the National Institute of Health, U.S.A; TIGR (The Institute for Genomic Research; http://www.tigr.org/db.shtml); the KEGG database (Kyoto Encyclopedia of Genes and Genomes; http://www.genome.ad.jp/kegg/); and, in the case of Prochlorococcus accession numbers, from CyanoBase (http://bacteria.kazusa.or.jp/cyanobase/). The accession numbers from NCBI are as provided in the database on Sep. 4, 2007.

Enzyme Classification Numbers (EC): The EC numbers provided throughout this description are derived from the KEGG Ligand database, maintained by the Kyoto Encyclopedia of Genes and Genomics, sponsored in part by the University of Tokyo. The EC numbers are as provided in the database on Sep. 4, 2007.

DNA: Deoxyribonucleic acid. DNA is a long chain polymer which includes the genetic material of most living organisms (some viruses have genes including ribonucleic acid, RNA). The repeating units in DNA polymers are four different nucleotides, each of which includes one of the four bases, adenine, guanine, cytosine and thymine bound to a deoxyribose sugar to which a phosphate group is attached.

Amino acid: An organic compound containing an amino group (NH2), a carboxylic acid group (COOH), and any of various side groups, especially any of the 20 compounds that have the basic formula NH2CHRCOOH, and that link together by peptide bonds to form proteins or that function as chemical messengers and as intermediates in metabolism. The arrangement of amino acids in a peptide is coded for by triplets of nucleotides or “codons” in DNA molecules. The term codon is also used for the corresponding (and complementary) sequences of three nucleotides in the mRNA into which the DNA sequence is transcribed.

Endogenous: As used herein with reference to a nucleic acid molecule and a particular cell or microorganism refers to a nucleic acid sequence or peptide that is in the cell and was not introduced into the cell using recombinant engineering techniques. For example, a gene that was present in the cell when the cell was originally isolated from nature. A gene is still considered endogenous if the control sequences (e.g., promoter or enhancer sequences that activate transcription or translation) have been altered through recombinant techniques.

Exogenous: As used herein with reference to a nucleic acid molecule and a particular cell or microorganism refers to a nucleic acid sequence or peptide that was not present in the cell when the cell was originally isolated from nature. For example, a nucleic acid that originated in a different microorganism and was engineered into an alternate cell using recombinant DNA techniques or other methods is an endogenous nucleic acid.

Expression: The process by which a gene's coded information is converted into the structures and functions of a cell, such as a protein, transfer RNA, or ribosomal RNA. Expressed genes include those that are transcribed into mRNA and then translated into protein and those that are transcribed into RNA but not translated into protein (for example, transfer and ribosomal RNAs).

Overexpression: When a gene is caused to be transcribed at an elevated rate compared to the endogenous transcription rate for that gene. In some examples, overexpression additionally includes an elevated rate of translation of the gene compared to the endogenous translation rate for that gene. Methods of testing for overexpression are well known in the art. For example, transcribed RNA levels can be assessed using reverse transcriptase polymerase chain reaction (RT-PCR) and protein levels can be assessed using sodium dodecyl sulfate polyacrylamide gel elecrophoresis (SDS-PAGE) analysis. Furthermore, a gene is considered to be overexpressed when it exhibits elevated activity compared to its endogenous activity, which may occur, for example, through reduction in concentration or activity of its inhibitor, or via expression of a mutant version with elevated activity. In preferred embodiments, when the host cell encodes an endogenous gene with a desired biochemical activity, it is useful to overexpress an exogenous gene, which allows for more explicit regulatory control in the fermentation and a means to potentially mitigate the effects of central metabolism regulation, which is focused around the native genes explicity.

Downregulation: When a gene is caused to be transcribed at a reduced rate compared to the endogenous gene transcription rate for that gene. In some examples, downregulation additionally includes a reduced level of translation of the gene compared to the endogenous translation rate for that gene. Methods of testing for downregulation are well known to those in the art, for example the transcribed RNA levels can be assessed using RT-PCR and proteins levels can be assessed using SDS-PAGE analysis.

Knock-out: A gene whose level of expression or activity has been reduced to zero. In some examples, a gene is knocked-out via deletion of some or all of its coding sequence. In other examples, a gene is knocked-out via introduction of one or more nucleotides into its open-reading frame, which results in translation of a non-sense or otherwise non-functional protein product.

Autotroph: Autotrophs (or autotrophic organisms) are organisms that produce complex organic compounds from simple inorganic molecules and an external source of energy, such as light (photoautotroph) or chemical reactions of inorganic compounds.

Heterotroph: Heterotrophs (or heterotrophic organisms) are organisms that, unlike autotrophs, cannot derive energy directly from light or from inorganic chemicals, and so must feed on organic carbon substrates. They obtain chemical energy by breaking down the organic molecules they consume. Heterotrophs include animals, fungi, and numerous types of bacteria.

Synthetophototroph: A natively heterotrophic organism that through recombinant DNA techniques has been engineered to express endogenous and exogenous biosynthetic pathways which allow it to grow in an autotrophic manner.

Hydrocarbon: generally refers to a chemical compound that consists of the elements carbon (C), optionally oxygen (O), and hydrogen (H).

Biosynthetic pathway: Also referred to as “metabolic pathway,” refers to a set of anabolic or catabolic biochemical reactions for converting (transmuting) one chemical species into another. For example, a hydrocarbon biosynthetic pathway refers to the set of biochemical reactions that convert inputs and/or metabolites to hydrocarbon product-like intermediates and then to hydrocarbons or hydrocarbon products. Anabolic pathways involve constructing a larger molecule from smaller molecules, a process requiring energy. Catabolic pathways involve the breaking down of larger molecules, often accompanied by the release of energy.

Cellulose: Cellulose [(C₆H₁₀O₅)ₙ] is a long-chain polysaccharide polymer of beta-glucose. It forms the primary structural component of plants and is not digestible by humans. Cellulose is a common material in plant cell walls and was first noted as such in 1838. It occurs naturally in almost pure form only in cotton fiber; in combination with lignin and any hemicellulose, it is found in all plant material.

Surfactants: Surfactants are substances capable of reducing the surface tension of a liquid in which they are dissolved. They are typically composed of a water-soluble head and a hydrocarbon chain or tail. The water soluble group is hydrophilic and can be either ionic or nonionic, and the hydrocarbon chain is hydrophobic.

Biofuel: A biofuel is any fuel that derives from a biological source.

Engineered nucleic acid: An “engineered nucleic acid” is a nucleic acid molecule that includes at least one difference from a naturally-occurring nucleic acid molecule. An engineered nucleic acid includes all exogenous modified and unmodified heterologous sequences (i.e., sequences derived from an organism or cell other than that harboring the engineered nucleic acid) as well as endogenous genes, operons, coding sequences, or non-coding sequences, that have been modified, mutated, or that include deletions or insertions as compared to a naturally-occurring sequence. Engineered nucleic acids also include all sequences, regardless of origin, that are linked to an inducible promoter or to another control sequence with which they are not naturally associated.

Light capture nucleic acid: A “light capture nucleic acid” refers to a nucleic acid that alone or in combination with another nucleic acid encodes one or more proteins that convert light energy (i.e. photons) into chemical energy such as a proton gradient, reducing power, or a molecule containing at least one high-energy phosphate bond such as ATP or GTP. Examples of a light capture nucleic acid include nucleic acids encoding light-activated proton pumps such as rhodopsin, xanthorhodopsin, proteorhodopsin and bacteriorhodopsin.

Carbon dioxide fixation pathway nucleic acid: A “carbon dioxide fixation pathway nucleic acid” refers to a nucleic acid that alone or in combination with another nucleic acid encodes a protein that enables autotrophic carbon fixation. Examples of a carbon dioxide fixation pathway nucleic acid includes nucleic acids encoding propionyl-CoA carboxylase, pyruvate synthase, and formate dehydrogenase.

NADH pathway nucleic acid: A “NADH pathway nucleic acid” refers to a nucleic acid that alone or in combination with another nucleic acid encodes a protein to maintain an appropriately balanced supply of reduced NAD for carrying out carbon fixation.

NADPH pathway nucleic acid: A “NADPH pathway nucleic acid” refers to a nucleic acid that alone or in combination with another nucleic acid encodes a protein to maintain an appropriately balanced supply of reduced NADPH for carrying out carbon fixation.

Acetyl-CoA flux nucleic acid: An “acetyl-CoA flux nucleic acid” refers to a nucleic acid that alone or in combination with another nucleic acid encodes a protein whose overexpression, downregulation, or inhibition results in an increase in acetyl-CoA produced over a unit of time. Example nucleic acids that may be overexpressed include pantothenate kinase and pyruvate dehydrogenase. Nucleic acids that may be downregulated, inhibited, or knocked-out include acyl coenzyme A dehydrogenase, biosynthetic glycerol 3-phosphate dehydrogenase, and lactate dehydrogenase.

### DETAILED DESCRIPTION OF THE INVENTION

E. coli Bacterial Strains and Propagation

The non-pathogenic lab adapted E. coli strains K-12 serves as the parental strain for subsequent genetic manipulation (available via The Coli Genetic Stock Center (CGSC) at Yale University). Alternately E. coli strains W or B can be used. Commercially-available derivatives, containing the T7 RNA polymerase gene under control of the lacUV5 promoter such as BL21(DE3) [F⁻ ompT hsdS (r_{B}⁻m_{B}⁻) gal dcm λDE3; Novagen, Madison Wis.] are useful for driving recombinant protein expression encoded on plasmids containing the T7 RNA polymerase promoter.

Light is delivered through a variety of mechanisms, including natural illumination (sunlight), standard incandescent, fluorescent, or halogen bulbs, or via propagation in specially-designed illuminated growth chambers (for example Model LI15 Illuminated Growth Chamber (Sheldon Manufacturing, Inc. Cornelius, Oreg.). For experiments requiring specific wavelengths and/or intensities, light is distributed via light emitting diodes (LEDs), in which wavelength spectra and intensity can be carefully controlled (Philips).

Carbon dioxide is supplied via inclusion of solid media supplements (i.e., sodium bicarbonate) or as a gas via its distribution into the growth incubator. Most experiments are performed using concentrated carbon dioxide gas, at concentrations between 10 and 30%, which is directly bubbled into the growth media at velocities sufficient to provide mixing for the organisms. When concentrated carbon dioxide gas is utilized, the gas originates in pure form from commercially-available cylinders, or preferentially from concentrated sources including offgas from coal plants, refineries, cement production facilities, natural gas facilities, breweries, and others.

### Plasmids

Plasmids relevant to genetic engineering typically include at least two functional elements 1) an origin of replication enabling propagation of the DNA sequence in the host organism, and 2) a selective marker (for example an antibiotic resistance marker conferring resistance to ampicillin, kanamycin, zeocin, chloramphenicol, tetracycline, spectinomycin, and the like). Plasmids are often referred to as “cloning vectors” when their primary purpose is to enable propagation of a desired heterologous DNA insert. Plasmids can also include cis-acting regulatory sequences to direct transcription and translation of heterologous DNA inserts (for example, promoters, transcription terminators, ribosome binding sites). Such plasmids are frequently referred to as “expression vectors.”

Table 1, below, lists preferred genes of interest to enable conversion of a heterotrophic organism into a photoautotroph.

TABLE 1
Overexpression genes of interest

| | | | Exemplary Gene | | Locus/ | |
|---|---|---|---|---|---|---|
| Module | Pathway/Module | EC (if relevant) | Name | Organism | Accession | Alternates |
| Light | Light PMF | | Proteorhodopsin | Uncultured | ABL60988 | Alternatives include |
| capture | | | | marine bacterium | | the HOT 0 ml gene |
| | | | | HF10_19P19 | | (AF349978), the |
| | | | | | | HOT 75m4 gene |
| | | | | | | (AF349981), the |
| | | | | | | palE6 gene |
| | | | | | | (AF350002), and |
| | | | | | | the SAR86 gene |
| | | | | | | from eBAC31A08 |
| | | | | | | (AAG10475). |
| Light | light PMF | | Bacteriorhodopsin | Halobacterium | NP_280292 | Alternatives include |
| capture | | | | species NRC-1 | | the Halobacterium |
| | | | | | | salinarum gene |
| | | | | | | (V00474) |
| Light | light PMF | | deltarhodopsin | Haloterrigena sp | AB009620 | Alternatives include |
| capture | | | | arg-4 | | the variant |
| | | | | | | described in Kamo N |
| | | | | | | et al, BBRC |
| | | | | | | 2006, from |
| | | | | | | Haloterrigena |
| | | | | | | turkmenica, which |
| | | | | | | differs only in 2 |
| | | | | | | positions compared |
| | | | | | | to AB009620 |
| Light | light PMF | | xanthorhodopsin | Salinibacter | ABC44767 | |
| capture | | | | ruber DSM | | |
| | | | | 13855 | | |
| Light | light PMF | | Opsin | Leptosphaeria | AAG01180 | |
| capture | | | | maculans | | |
| Light | Retinal biosynthesis | 5.3.3.2 | Isopentenyl- | Uncultured | ABL60982 | Alternatives include |
| capture | | | diphosphate delta- | marine bacterium | | E. coli (JW2857) |
| | | | isomerase | HF10_19P19 | | and Rhodococcus |
| | | | | | | capsulatus |
| | | | | | | (CAA77535.1) |
| Light | Retinal biosynthesis | 1.14.99.36 | 15,15′-beta- | Uncultured | ABL60983 | Homo sapiens |
| capture | | | carotene | marine bacterium | | (AAG15380) and |
| | | | dioxygenase | HF10_19P19 | | Mus musculus |
| | | | | | | (AJ278064) |
| Light | Retinal biosynthesis | | Lycopene cyclase | Uncultured | ABL60984 | cruA gene from |
| capture | | | | marine bacterium | | Synechococcus sp |
| | | | | HF10_19P19 | | PCC 7002 |
| | | | | | | (EF529626) and |
| | | | | | | cruP from same |
| | | | | | | species |
| | | | | | | (EF529627), and |
| | | | | | | crtY from |
| | | | | | | Streptomyces |
| | | | | | | coelicolor |
| | | | | | | (SCJ12.03, or |
| | | | | | | NC_003888.3) |
| Light | Retinal biosynthesis | 2.5.1.32 | Phytoene synthase | Uncultured | ABL60985 | Streptomyces |
| capture | | | | marine bacterium | | coelicolor A3(2) |
| | | | | HF10_19P19 | | [locus SCO0187] or |
| | | | | | | Prochlorococcus |
| | | | | | | marinus crtB |
| | | | | | | [Pro0166 or |
| | | | | | | NC_005042.1] |
| Light | Retinal biosynthesis | | Phytoene | Uncultured | ABL60986 | Prochlorococcus |
| capture | | | dehydrogenase | marine bacterium | | marinus [Pro0167] |
| | | | | HF10_19P19 | | or |
| | | | | | | Thermosynechococcus |
| | | | | | | elongatus BP-1 |
| | | | | | | [tll1561] |
| Light | Retinal biosynthesis | | Geranylgeranyl | Uncultured | ABL60987 | Rhodobacter |
| capture | | | pyrophosphate | marine bacterium | | sphaeroides 2.4.1 |
| | | | synthetase | HF10_19P19 | | crtE gene |
| | | | | | | [RSP_0265] and |
| | | | | | | Arabidopsis |
| | | | | | | thaliana GGPS3 |
| | | | | | | [AT3G14550] |
| Light | Salinixanthin | | beta-carotene | Salinibacter | SRU_1502 | Other crtO genes |
| capture | | | ketolase | ruber DSM | | include |
| | | | | 13855 | | Rhodococcus |
| | | | | | | erythropolis |
| | | | | | | (AY705709), |
| | | | | | | Deinococcus |
| | | | | | | radiodurans R1 |
| | | | | | | (NP_293819).), and |
| | | | | | | Gloeobacter |
| | | | | | | violaceus PCC 7421 |
| | | | | | | [gvip239]. |
| Light | Green-sulfur | | photosystem P840 | Chlorobium | CT2020 | |
| capture | photosystem I | | reaction center large | tepidum | | |
| | | | subunit, pscA | | | |
| Light | Green-sulfur | | photosystem P840 | Chlorobium | CT2019 | |
| capture | photosystem I | | reaction center iron- | tepidum | | |
| | | | sulfur protein, pscB | | | |
| Light | Green-sulfur | | photosystem P840 | Chlorobium | CT1639 | |
| capture | photosystem I | | reaction center | tepidum | | |
| | | | cytochrome c-551, | | | |
| | | | pscC | | | |
| Light | Green-sulfur | | photosystem P840 | Chlorobium | CT0641 | |
| capture | photosystem I | | reaction center | tepidum | | |
| | | | protein, pscD | | | |
| Light | Green-sulfur | | bacteriochlorophyl | Chlorobium | CT1499 | |
| capture | photosystem I | | a binding protein, | tepidum | | |
| | | | Fenna-Mathews- | | | |
| | | | Olson protein, FMO | | | |
| Light | Cyanobacteria | | Photosystem I P700 | Prochlorococcus | Pro1672 | |
| capture | photosystem I | | chlorophyll A | marinus | | |
| | | | apoproptein A1, | | | |
| | | | psaA | | | |
| Light | Cyanobacteria | | Photosystem I P700 | Prochlorococcus | Pro1673 | |
| capture | photosystem I | | chlorophyll A | marinus | | |
| | | | apoproptein A2, | | | |
| | | | psaB | | | |
| Light | Cyanobacteria | | Photosystem I iron- | Prochlorococcus | Pro1767 | |
| capture | photosystem I | | sulfur center | marinus | | |
| | | | subunity VII, psaC | | | |
| Light | Cyanobacteria | | Photosystem I | Prochlorococcus | Pro1733 | |
| capture | photosystem I | | reaction center | marinus | | |
| | | | subunit II, psaD | | | |
| Light | Cyanobacteria | | Photosystem I | Prochlorococcus | Pro0371 | |
| capture | photosystem I | | reaction centre | marinus | | |
| | | | subunit IV PsaE | | | |
| Light | Cyanobacteria | | Photosystem I | Prochlorococcus | Pro0466 | |
| capture | photosystem I | | reaction centre | marinus | | |
| | | | subunit IX PsaJ | | | |
| Light | Cyanobacteria | | Photosystem I | Prochlorococcus | Pro0467 | |
| capture | photosystem I | | reaction centre | marinus | | |
| | | | subunit III precursor | | | |
| | | | (PSI-F | | | |
| Light | Cyanobacteria | | Photosystem I | Prochlorococcus | Pro0541 | |
| capture | photosystem I | | reaction centre | marinus | | |
| | | | subunit XII PsaM | | | |
| Light | Cyanobacteria | | Photosystem I | Prochlorococcus | Pro0929 | |
| capture | photosystem I | | reaction center | marinus | | |
| | | | subunit PsaK | | | |
| Light | Cyanobacteria | | Photosystem I | Prochlorococcus | Pro1253 | |
| capture | photosystem I | | assembly protein | marinus | | |
| Light | Cyanobacteria | | Photosystem I | Prochlorococcus | Pro1678 | |
| capture | photosystem I | | subunit VIII PsaI | marinus | | |
| Light | Cyanobacteria | | Photosystem I | Prochlorococcus | Pro1679 | |
| capture | photosystem I | | reaction centre | marinus | | |
| | | | subunit XI PsaL | | | |
| Light | Cyanobacteria | | Photosystem II | Prochlorococcus | Pro0076 | |
| capture | photosystem II | | protein X PsbX | marinus | | |
| Light | Cyanobacteria | | Photosystem II | Prochlorococcus | Pro0252 | |
| capture | photosystem II | | reaction center D1 | marinus | | |
| Light | Cyanobacteria | | Photosystem II | Prochlorococcus | Pro0257 | |
| capture | photosystem II | | manganese- | marinus | | |
| | | | stabilizing protein | | | |
| | | | PsbO | | | |
| Light | Cyanobacteria | | Photosystem II 10 kDa | Prochlorococcus | Pro0283 | |
| capture | photosystem II | | phosphoprotein | marinus | | |
| | | | PsbH | | | |
| Light | Cyanobacteria | | Photosystem II | Prochlorococcus | Pro0284 | |
| capture | photosystem II | | reaction center N | marinus | | |
| | | | protein PsbN | | | |
| Light | Cyanobacteria | | Photosystem II | Prochlorococcus | Pro0285 | |
| capture | photosystem II | | protein PsbI | marinus | | |
| Light | Cyanobacteria | | Photosystem II | Prochlorococcus | Pro0304 | |
| capture | photosystem II | | protein PsbK | marinus | | |
| Light | Cyanobacteria | | Photosystem II | Prochlorococcus | Pro0327 | |
| capture | photosystem II | | stability/assembly | marinus | | |
| | | | factor | | | |
| Light | Cyanobacteria | | Cytochrome b559 | Prochlorococcus | Pro0328 | |
| capture | photosystem II | | alpha subunit PsbE | marinus | | |
| Light | Cyanobacteria | | Cytochrome b559 | Prochlorococcus | Pro0329 | |
| capture | photosystem II | | beta chain PsbF | marinus | | |
| Light | Cyanobacteria | | Photosystem II | Prochlorococcus | Pro0330 | |
| capture | photosystem II | | protein L PsbL | marinus | | |
| Light | Cyanobacteria | | Photosystem II | Prochlorococcus | Pro0331 | |
| capture | photosystem II | | protein J PsbJ | marinus | | |
| Light | Cyanobacteria | | Possible PucC | Prochlorococcus | Pro0346 | |
| capture | photosystem II | | protein | marinus | | |
| Light | Cyanobacteria | | Photosystem II | Prochlorococcus | Pro0353 | |
| capture | photosystem II | | reaction center T | marinus | | |
| | | | PsbT | | | |
| Light | Cyanobacteria | | Photosystem II | Prochlorococcus | Pro0354 | |
| capture | photosystem II | | chlorophyll | marinus | | |
| | | | a-binding protein | | | |
| | | | CP47 homolog | | | |
| Light | Cyanobacteria | | Photosystem II | Prochlorococcus | Pro0357 | |
| capture | photosystem II | | protein M PsbM | marinus | | |
| Light | Cyanobacteria | | Photosystem II | Prochlorococcus | Pro0507 | |
| capture | photosystem II | | protein Psb27 | marinus | | |
| Light | Cyanobacteria | | Photosystem II | Prochlorococcus | Pro0586 | |
| capture | photosystem II | | protein Y PsbY | marinus | | |
| Light | Cyanobacteria | | Photosystem II | Prochlorococcus | Pro0771 | |
| capture | photosystem II | | reaction centre W | marinus | | |
| | | | protein | | | |
| Light | Cyanobacteria | | Photosystem II | Prochlorococcus | Pro1097 | |
| capture | photosystem II | | protein P PsbP | marinus | | |
| Light | Cyanobacteria | | Flavodoxin, IsiB | Prochlorococcus | Pro1164 | |
| capture | photosystem II | | | marinus | | |
| Light | Cyanobacteria | | Photosystem II | Prochlorococcus | Pro1254 | |
| capture | photosystem II | | reaction center D2 | marinus | | |
| Light | Cyanobacteria | | Photosystem II | Prochlorococcus | Pro1255 | |
| capture | photosystem II | | chlorophyll a- | marinus | | |
| | | | binding protein | | | |
| | | | CP43 homolog | | | |
| Light | Cyanobacteria | | Homolog of PsbF | Prochlorococcus | Pro1494 | |
| capture | photosystem II | | protein | marinus | | |
| Carbon | 3-Hydroxypropionate | 6.4.1.2 | Acetyl-CoA | Escherichia coli | AAA70370 | Homo sapiens |
| Fixation | cycle | | carboxylase | | | [ACACA, |
| | | | (subunit alpha) | | | NC000017.9] |
| Carbon | 3-Hydroxypropionate | 6.4.1.2 | Acetyl-CoA | Escherichia coli | AAA23807 | Arabidopsis |
| Fixation | cycle | | carboxylase | | | thaliana |
| | | | (subunit beta) | | | [AtCg00500] |
| Carbon | 3-Hydroxypropionate | 6.4.1.2 | Biotin-carboxyl | Escherichia coli | JW3223 | Bacillus halodurans |
| Fixation | cycle | | carrier protein | | | [BH1132], Vibrio |
| | | | (accB) | | | cholerae |
| | | | | | | [EAZ76879.1 or |
| | | | | | | A5E_0311] |
| Carbon | 3-Hydroxypropionate | 6.4.1.2 | biotin-carboxylase | Escherichia coli | AAA23748 | Photobacterium |
| Fixation | cycle | | | | | profundum 3TCK |
| | | | | | | [EAS42088.1 or |
| | | | | | | 90325619] |
| Carbon | 3-Hydroxypropionate | 1.1.1.59 | malonyl-CoA | Chloroflexus | AY530019 | |
| Fixation | cycle | | reductase | aurantiacus | | |
| Carbon | 3-Hydroxypropionate | | 3- | Chloroflexus | AF445079 | AMP-dependent |
| Fixation | cycle | | hydroxypropionyl- | aurantiacus | | synthetase and |
| | | | CoA synthase | | | ligase |
| | | | | | | [ABQ91563.1] from |
| | | | | | | Roseiflexus sp RS- |
| | | | | | | 1. |
| Carbon | 3-Hydroxypropionate | 6.4.1.3 | propionyl-CoA | Roseobacter | RD1_2032 | Homo sapiens |
| Fixation | cycle | | carboxylase | denitrificans | | mitochondrial |
| | | | (subunit alpha) | | | PCCA gene |
| | | | | | | [X14608]. Mus |
| | | | | | | musculus PCCA |
| | | | | | | gene [AY046947] |
| Carbon | 3-Hydroxypropionate | 6.4.1.3 | propionyl-CoA | Roseobacter | RD1_2028 | Rhodococcus |
| Fixation | cycle | | carboxylase | denitrificans | | erythropolis |
| | | | (subunit beta) | | | [AAB80770.1], |
| | | | | | | Homo sapiens |
| | | | | | | mitochondrial |
| | | | | | | PCCB [X73424] |
| Carbon | 3-Hydroxypropionate | 5.1.99.1 | methylmalonyl- | Rhodobacter | CP000661 | Homo sapiens |
| Fixation | cycle | | CoA epimerase | sphaeroides | | MCEE [AF364547] |
| Carbon | 3-Hydroxypropionate | 5.1.99.2 | methylmalonyl- | Escherichia coli | NC000913.2 | Homo sapiens MUT |
| Fixation | cycle | | CoA mutase | | | [M65131] |
| Carbon | 3-Hydroxypropionate | | succinyl-CoA:L- | Chloroflexus | DQ472736.1 | L-carnitine |
| Fixation | cycle | | malate CoA | aurantiacus | | dehydratase/bile |
| | | | transferase (subunit | | | acid-inducible |
| | | | alpha) | | | protein F from |
| | | | | | | Chloroflexus |
| | | | | | | aggregans DSM |
| | | | | | | 0485 |
| | | | | | | [ZP_01516527.1 or |
| | | | | | | EAV09800.1] |
| Carbon | 3-Hydroxypropionate | | succinyl-CoA:L- | Chloroflexus | DQ472737.1 | L-carnitine |
| Fixation | cycle | | malate CoA | aurantiacus | | dehydratase/bile |
| | | | transferase (subunit | | | acid-inducible |
| | | | beta) | | | protein F from |
| | | | | | | Chloroflexus |
| | | | | | | aggregans DSM |
| | | | | | | 9485 |
| | | | | | | [ZP_01516526.1 or |
| | | | | | | EAV09799.1] |
| Carbon | 3-Hydroxypropionate | 1.3.1.6 | fumarate reductase - | Escherichia coli | AAA23437.1 | Salmonella enterica |
| Fixation | cycle | | frdA-flavoprotein | | | subsp. enterica |
| | | | subunit | | | serovar fumarate |
| | | | | | | reductase |
| | | | | | | NP_458782.1 or |
| | | | | | | Klebsiella |
| | | | | | | pneumoniae |
| | | | | | | ABR79907.1 |
| Carbon | 3-Hydroxypropionate | 1.3.1.6 | fumarate reductase | Escherichia coli | EAY46226.1 | Salmonella |
| Fixation | cycle | | iron-sulfur subunit- | | | typhimurium LT2 |
| | | | frdb | | | succinate |
| | | | | | | dehydrogenase |
| | | | | | | [NP_463206.1] |
| Carbon | 3-Hydroxypropionate | 1.3.1.6 | g15 subunit | Escherichia coli | NP_290787.1 | Shigella flexneri 2a |
| Fixation | cycle | | [fumarate reductase | | | str. 301 |
| | | | subunit c] | | | [NP_710021.1], |
| | | | | | | Klebsiella |
| | | | | | | pneumoniae |
| | | | | | | ABR79905.1] |
| Carbon | 3-Hydroxypropionate | 1.3.1.6 | g13 subunit | Escherichia coli | NP_757086.1 | Salmonella enterica |
| Fixation | cycle | | [fumarate reductase | | | [YP_153210.1], |
| | | | subunit D] | | | Photorhabdus |
| | | | | | | luminescens |
| | | | | | | [NP_931317.1 |
| Carbon | 3-Hydroxypropionate | 4.2.1.2 | fumarate hydratase - | Escherichia coli | CAA25204 | Alternates include |
| Fixation | cycle | | class I aerobic | | | E. coli class I |
| | | | (fumA) | | | anaerobic fumarate |
| | | | | | | hydratase (fumB) |
| | | | | | | AAA23827 or class |
| | | | | | | II (fumC) |
| | | | | | | CAA27698 |
| Carbon | 3-Hydroxypropionate | 4.1.3.24 | L-malyl-CoA lyase | Roseobacter | NC_008209.1 | Silicibacter |
| Fixation | cycle | | | denitrificans | | pomeroyi DSS-3 |
| | | | | | | citrate lyase |
| | | | | | | putative |
| | | | | | | [YP_166806.1] and |
| | | | | | | alpha |
| | | | | | | proteobacterium |
| | | | | | | HTCC2255 |
| | | | | | | [ZP_01447127.1] |
| Carbon | Reductive TCA | 2.3.3.8 | ATP-citrate lyase, | Chlorobium | CT1089 | Chlorobium |
| Fixation | | | subunit 1 | tepidum | | limicola |
| | | | | | | [BAB21375.1], |
| | | | | | | Chlorobium |
| | | | | | | ferrooxidans DSM |
| | | | | | | 13031 |
| | | | | | | [ZP_01385848.1] |
| Carbon | Reductive TCA | 2.3.3.8 | ATP-citrate lyase, | Chlorobium | CT1088 | Chlorobium |
| Fixation | | | subunit 2 | tepidum | | limicola |
| | | | | | | [BAB21376.1], |
| | | | | | | Chlorobium |
| | | | | | | phaeobacteroides |
| | | | | | | [YP_911761.1], |
| | | | | | | Chlorobium |
| | | | | | | ferrooxidans |
| | | | | | | [ZP_01385849.1]. |
| Carbon | Reductive TCA | | citryl-CoA synthase | Hydrogenobacter | BAD17844 | Aquifex aeolicus |
| Fixation | | | (large subunit) | thermophilus | | [O67330], |
| | | | | | | Leptospirillum sp. |
| | | | | | | Group II UBA |
| | | | | | | [A3ERU1] |
| Carbon | Reductive TCA | | citryl-CoA synthase | Hydrogenobacter | BAD17846 | Aquifex aeolicus |
| Fixation | | | (small subunit) | thermophilus | | [NP_214297.1], |
| | | | | | | Leptospirillum sp |
| | | | | | | Group II UBA |
| | | | | | | [EAY57418.1] |
| Carbon | Reductive TCA | | citryl-CoA ligase | Hydrogenobacter | BAD17841 | Aquifex aeolicus |
| Fixation | | | | thermophilus | | [NP_213101.], |
| | | | | | | Hydrogenobacter |
| | | | | | | hydrogenophilus |
| | | | | | | [ABI50086.1] |
| Carbon | Reductive TCA | 1.1.1.37 | malate | Chlorobium | CAA56810 | Prosthecochloris |
| Fixation | | | dehydrogenase | tepidum | | vibrioformis |
| | | | | | | [CAA56809.1], |
| | | | | | | Pelodictyon |
| | | | | | | luteolum DSM 273 |
| | | | | | | [YP_375410.1] |
| Carbon | Reductive TCA | 4.2.1.2 | fumarase hydratase | Escherichia coli | JW1604 | Alternatives include |
| Fixation | | | (aerobic isozyme, | | | E. coli class I |
| | | | fumA) | | | anaerobic isozyme |
| | | | | | | fumB (JW4083) and |
| | | | | | | class II fumC |
| | | | | | | (JW1603) |
| Carbon | Reductive TCA | 1.3.99.1 | succinate | Escherichia coli | NP_415251 | Enterobacter sp. |
| Fixation | | | dehydrogenase | | | 638 |
| | | | (flavoprotein | | | [YP_001175956.1], |
| | | | subunit - SdhA) | | | Serratia |
| | | | | | | proteamaculans |
| | | | | | | [ZP_01538596.1] |
| Carbon | Reductive TCA | 1.3.99.1 | SdhB iron-sulfur | Escherichia coli | NP_415252 | Salmonella enterica |
| Fixation | | | subunit | | | [YP_151223.1], |
| | | | | | | Yersinia |
| | | | | | | enterocolitica |
| | | | | | | [YP_001007133.1] |
| Carbon | Reductive TCA | 1.3.99.1 | SdhC membrane | Escherichia coli | NP_415249 | Enterobacter sp. |
| Fixation | | | anchor subunit | | | 638 [ABP59903.1], |
| | | | | | | Yersinia |
| | | | | | | frederiksenii |
| | | | | | | [ZP_00828037.1] |
| Carbon | Reductive TCA | 1.3.99.1 | SdhD membrane | Escherichia coli | NP_415250 | Enterobacter sp. |
| Fixation | | | anchor subunit | | | 638 |
| | | | | | | [YP_001175955.1], |
| | | | | | | Klebsiella |
| | | | | | | pneumoniae |
| | | | | | | [YP_001334402.1] |
| Carbon | Reductive TCA | 6.2.1.5 | succinyl-CoA | Escherichia coli | AAA23900 | |
| Fixation | | | synthetase subunit | | | |
| | | | alpha (sucD) | | | |
| Carbon | Reductive TCA | 6.2.1.5 | succinyl-CoA | Escherichia coli | AAA23899 | |
| Fixation | | | synthetase subunit | | | |
| | | | beta (sucC) | | | |
| Carbon | Reductive TCA | 1.2.7.3 | alpha-ketoglutarate | Hydrogenobacter | AB046568: | Alternative enzyme |
| Fixation | | | subunit alpha - korA | thermophilus | 46-1869 | from Chlorobium |
| | | | | | | limicola DSM 245. |
| | | | | | | 4 subunit enzyme |
| | | | | | | with accession |
| | | | | | | numbers |
| | | | | | | EAM42575, |
| | | | | | | EAM42574, |
| | | | | | | EAM42853, |
| | | | | | | EAM42852. |
| Carbon | Reductive TCA | 1.2.7.3 | alpha-ketoglutarate | Hydrogenobacter | AB046568: | There is another 5- |
| Fixation | | | subunit beta - korB | thermophilus | 1883-2770 | subunit OGOR |
| | | | | | | cluster in the same |
| | | | | | | bacteria. Yun NR et |
| | | | | | | al. BBRC (2002). |
| | | | | | | A novel five- |
| | | | | | | subunit-type 2- |
| | | | | | | oxoglutalate:ferredoxin |
| | | | | | | oxidoreductases |
| | | | | | | from |
| | | | | | | Hydrogenobacter |
| | | | | | | thermophilus TK-6. |
| | | | | | | 292(1): 280-6. |
| | | | | | | Genes are |
| | | | | | | forDABGE |
| Carbon | Reductive TCA | 1.1.1.42 | Isocitrate | Chlorobium | EAM42635 | Another exemplary |
| Fixation | | | dehydrogenase - | limicola | | enzyme is |
| | | | NADP dependent | | | Synechococcus sp |
| | | | | | | WH 8102, icd, |
| | | | | | | accession |
| | | | | | | CAE06681 |
| Carbon | Reductive TCA | 1.1.1.41 | isocitrate | Saccharomyces | YNL037C | |
| Fixation | | | dehydrogenase - | cerevisiae | | |
| | | | NAD depend. | | | |
| | | | Subunit 1 | | | |
| Carbon | Reductive TCA | 1.1.1.41 | isocitrate | Saccharomyces | YOR136W | |
| Fixation | | | dehydrogenase - | cerevisiae | | |
| | | | NAD depend. | | | |
| | | | Subunit 2 | | | |
| Carbon | Reductive TCA | 4.2.1.3 | aconitate hydratase | Escherichia coli | b1276 | |
| Fixation | | | 1 (acnA) | | | |
| Carbon | Reductive TCA | 4.2.1.3 | aconitate hydratase | Escherichia coli | b0118 | |
| Fixation | | | 2 (acnB) | | | |
| Carbon | Reductive TCA | 1.2.7.1 | Pyruvate synthase, | Clostridium | AA036986 | |
| Fixation | | | subunit A porA | tetani E88 | | |
| Carbon | Reductive TCA | 1.2.7.1 | Pyruvate synthase, | Clostridium | AA036985 | |
| Fixation | | | subunit B porB | tetani E88 | | |
| Carbon | Reductive TCA | 1.2.7.1 | Pyruvate synthase, | Clostridium | AA036988 | |
| Fixation | | | subunit C porC | tetani E88 | | |
| Carbon | Reductive TCA | 1.2.7.1 | Pyruvate synthase, | Clostridium | AA036987 | |
| Fixation | | | subunit D porD | tetani E88 | | |
| Carbon | Reductive TCA | 2.7.9.2 | Phosphoenolpyruvate | Escherichia coli | AAA2431 | Another exemplary |
| Fixation | | | synthase - ppsA | | | enzyme is Aquifex |
| | | | | | | aeolicus VF5 ppsA |
| | | | | | | (locus AAC07865). |
| Carbon | Reductive TCA | 4.1.1.31 | PEP carboxylase, | Escherichia coli | CAA29332 | |
| Fixation | | | ppC | | | |
| Carbon | Woods-Ljungdahl | 1.2.1.4.3 | NADP-dependent | Moorella | AAB18330 | |
| Fixation | | | formate | thermoacetica | | |
| | | | dehydrogenase - | | | |
| | | | subunit A Mt-fdhA | | | |
| Carbon | Woods-Ljungdahl | 1.2.1.4.3 | NADP-dependent | Moorella | AAB18329 | |
| Fixation | | | formate | thermoacetica | | |
| | | | dehydrogenase - | | | |
| | | | subunit B Mt-fdhB | | | |
| Carbon | Woods-Ljungdahl | 6.3.4.3 | formate | Clostridium | M21507 | Alternative sources |
| Fixation | | | tetrahydrofolate | acidi-urici | | include locus |
| | | | ligase | | | AAB49329 from |
| | | | | | | Streptococcus |
| | | | | | | mutans (Swiss-Prot |
| | | | | | | entry Q59925) or |
| | | | | | | the Q8XHL4 |
| | | | | | | protein from |
| | | | | | | Clostridium |
| | | | | | | perfingens (locus |
| | | | | | | BA000016) |
| Carbon | Woods-Ljungdahl | 3.5.4.9 and | Methenyltetrahydro | Escherichia coli | AAA23803 | Alternative sources |
| Fixation | | 1.5.1.5 | folate | | | include locus |
| | | | cyclohydrolase | | | ABC19825 (folD) |
| | | | | | | from Moorella |
| | | | | | | thermoacetica, |
| | | | | | | locus AAO36126 |
| | | | | | | from Clostridium |
| | | | | | | tetani, and locus |
| | | | | | | BAB81529 from |
| | | | | | | Clostridium |
| | | | | | | perfingens All are |
| | | | | | | bifunctional folD |
| | | | | | | enzymes. |
| Carbon | Woods-Ljungdahl | 1.5.1.20 | methylene | Escherichia coli | CAA24747 | Alternative sources |
| Fixation | | | tetrahydrofolate | | | include locus |
| | | | reductase, metF | | | AAC23094 from |
| | | | | | | Haemophilus |
| | | | | | | influenzae, or locus |
| | | | | | | CAA30531 from |
| | | | | | | Salmonella |
| | | | | | | typhimurium. |
| Carbon | Woods-Ljungdahl | | 5- | Moorella | AAA53548 | Another exemplary |
| Fixation | | | methyltetrahydrofolate | thermoacetica | | enzyme is acsE |
| | | | corrinoid/iron | | | from |
| | | | sulfur protein | | | Carboxydothermus |
| | | | methyltransferase, | | | hydrogenoformas |
| | | | acsE | | | locus CP000141 |
| Carbon | Woods-Ljungdahl | 1.2.7.4 and | Carbon monoxide | Moorella | AAA23229 | |
| Fixation | | 1.2.99.2 | dehydrogenase/acetyl- | thermoacetica | | |
| | | | CoA synthase - | | | |
| | | | subunit alpha | | | |
| Carbon | Woods-Ljungdahl | 1.2.7.4 and | Carbon monoxide | Moorella | AAA23228 | |
| Fixation | | 1.2.99.2 | dehydrogenase/acetyl- | thermoacetica | | |
| | | | CoA synthase - | | | |
| | | | subunit beta | | | |
| Carbon | Glyoxylate Shunt | 2.3.3.9 | malate synthase - | Escherichia coli | JW3974 | E. coli encodes an |
| Fixation | | | aceB | | | alternate malate |
| | | | | | | synthase enzyme, |
| | | | | | | the JW2943 locus |
| | | | | | | malate synthase G |
| | | | | | | (glcB) |
| Carbon | Glyoxylate Shunt | 4.1.3.1 | isocitrate lyase - | Escherichia coli | JW3975 | |
| Fixation | | | aceA | | | |
| Carbon | Glyoxylate Shunt | 1.1.1.37 | malate | Escherichia coli | JW3205 | |
| Fixation | | | dehydrogenase | | | |
| Carbon | Gluconeogenesis | 6.4.4.1 | pyruvate | Saccharomyces | YGL062W | |
| Fixation | | | carboxylase | cerevisiae | | |
| Carbon | Gluconeogenesis | 4.1.1.49 | phosphoenolpyruvate | Escherichia coli | JW3366 | |
| Fixation | | | carboxykinase | | | |
| Carbon | Gluconeogenesis | 3.1.3.11 | fructose-1,6- | Escherichia coli | JW4191 | |
| Fixation | | | bisphosphatase | | | |
| Carbon | Gluconeogenesis | 3.1.3.68 | glucose-6- | Saccharomyces | YHR044C | Saccharomyces |
| Fixation | | | phosphatase - dog1 | cerevisiae | | cerevisiae encodes a |
| | | | | | | second glucose-6- |
| | | | | | | phosphatase, |
| | | | | | | YHR043C locus, |
| | | | | | | dog2 |
| Carbon | pyruvate synthesis | 1.2.7.1 | pyruvate | Moorella | Moth_0064 | |
| Fixation | | | ferredoxin:oxidoreductase | thermoaceticum | | |
| | | | with | | | |
| | | | pyruvate synthase | | | |
| | | | activity | | | |
| Carbon | Reductive pentose | | fructose-1,6- | Synechococcus | ZP_01124026 | |
| Fixation | phosphate | | bisphosphatase | sp. WH 7805 | | |
| | | | (FBPase) and | | | |
| | | | sedoheptulose-1,7- | | | |
| | | | bisphosphatase | | | |
| | | | (SBPase), | | | |
| | | | bifunctional, cbbF | | | |
| Carbon | Reductive pentose | 1.2.1.13 | glyceraldehyde-3- | Prochlorococcus | NP_875968 | |
| Fixation | phosphate | | phosphate | marinus | | |
| | | | dehydrogenase | | | |
| | | | (GAPDH), cbbG | | | |
| Carbon | Reductive pentose | 2.7.1.19 | phosphoribulokinase | Prochlorococcus | NP_894365 | |
| Fixation | phosphate | | (PRK), cbbP | marinus | | |
| Carbon | Reductive pentose | | CP12 | Thermosynechococcus | BAC09372 | Chlamydomonas |
| Fixation | phosphate | | | elongatus | | reinhardtii locus |
| | | | | BP-1 | | CAO03469; |
| | | | | | | Synechococcus |
| | | | | | | elongatus PCC |
| | | | | | | 6301 locus |
| | | | | | | BAD79451 |
| Carbon | Reductive pentose | 2.2.1.1 | transketolase, cbbT | Synechocystis sp. | BAD79173.1 | |
| Fixation | phosphate | | | PCC 6301 | | |
| Carbon | Reductive pentose | 4.1.2.13 | fructose 1,6- | Synechocystis sp. | BAA10184 | |
| Fixation | phosphate | | bisphosphate | PCC 6803 | | |
| | | | aldolase, cbbA | | | |
| Carbon | Reductive pentose | 5.1.3.1 | pentose-5- | Synechocystis sp. | BAD79110 | |
| Fixation | phosphate | | phosphate-3- | PCC 6301 | | |
| | | | epimerase, cbbE | | | |
| Carbon | Reductive pentose | 5.3.1.6 | ribose 5-phosphate | Synechococcus | BAD79129 | |
| Fixation | phosphate | | isomerase | elongatus PCC | | |
| | | | | 6301 | | |
| Carbon | Reductive pentose | 2.7.2.3 | phosphoglycerate | Synechococcus | BAD78623 | |
| Fixation | phosphate | | kinase | elongatus PCC | | |
| | | | | 6301 | | |
| Carbon | Reductive pentose | 5.3.1.1 | triosephosphate | Synechocystis sp | Q59994 | |
| Fixation | phosphate | | isomerase, tpiA | PCC 6803 | | |
| Carbon | Reductive pentose | 4.1.1.39 | Ribulose-1,5- | Synechococcus | AAB48081.1 | |
| Fixation | phosphate | | bisphosphate | sp WH7803 | | |
| | | | carbyxlase/oxygenase | | | |
| | | | (RubisCo) - small | | | |
| | | | subunit - cbbS | | | |
| Carbon | Reductive pentose | 4.1.1.39 | Ribulose-1,5- | Synechococcus | AAB8080.1 | |
| Fixation | phosphate | | bisphosphate | sp WH7803 | | |
| | | | carbyxlase/oxygenase | | | |
| | | | (RubisCo) - large | | | |
| | | | subunit cbbL | | | |
| Carbon | Reductive pentose | | Rubisco activase | Synechococcus | ABC98646 | |
| Fixation | phosphate | | | sp. JA-3-3Ab | | |
| Reducing | NADH | 1.1.1.41 | NAD⁺-dependent | Saccharomyces | YNL037C | |
| power | | | isocitrate | cerevisiae | | |
| | | | dehydrogenase - | | | |
| | | | idh1 | | | |
| Reducing | NADH | 1.1.1.41 | NAD⁺-dependent | Saccharomyces | YOR136W | |
| power | | | isocitrate | cerevisiae | | |
| | | | dehydrogenase - | | | |
| | | | idh2 | | | |
| Reducing | NADH | 1.1.1.37 | malate | Escherichia coli | JW3205 | |
| power | | | dehydrogenase | | | |
| Reducing | NADPH | 1.6.1.1 | soluble pyridine | Escherichia coli | NP_418397.2 | Alternates include |
| power | | | nucleotide | | | Shigella flexneri |
| | | | transhydrogenase | | | locus Q83MI1 |
| Reducing | NADH | | NADH:ubiquinone | Rhodobacter | AF029365 | Consists of 14 nuo |
| power | | | oxidoreductase - | capsulatus | | genes A-N and 7 |
| | | | OPERON (a-n), | | | ORFs of unknown |
| | | | note not listing | | | function |
| | | | genes individually | | | |
| Reducing | NADPH | 1.1.1.49 | glucose-6- | Escherichia coli | JW1841 | |
| power | | | phosphate | | | |
| | | | dehydrogenase, zwf | | | |
| Reducing | NADPH | 3.1.1.31 | 6- | Escherichia coli | JW0750 | |
| power | | | phosphogluconolactonase - | | | |
| | | | pgi | | | |
| Reducing | NADPH | 1.1.1.44 | 6-phosphogluconate | Escherichia coli | JW2011 | |
| power | | | dehydrogenase, | | | |
| | | | gnd | | | |
| Reducing | NADPH | 1.1.1.42 | NADP-dependent | Escherichia coli | JW1122 | |
| power | | | isocitrate | | | |
| | | | dehydrogenase | | | |
| Reducing | NADPH | 1.1.1.40 | NADP-dependent | Escherichia coli | JW2447 | |
| power | | | malic enyme | | | |
| Reducing | NADPH | 1.6.1.1 | soluble pyridine | Escherichia coli | NP_418397.2 | Alternates include |
| power | | | nucleotide | | | Shigella flexneri |
| | | | transhydrogenase | | | locus Q83MI1 |
| Reducing | NADPH | | membrane-bound | Escherichia coli | JW1595 | |
| power | | | pyridine nucleotide | | | |
| | | | transhydrogenase, | | | |
| | | | subunit alpha, pntA | | | |
| Reducing | NADPH | | membrane-bound | Escherichia coli | JW1594 | |
| power | | | pyridine nucleotide | | | |
| | | | transhydrogenase, | | | |
| | | | subunit beta, pntB | | | |

The nucleotide sequences for the indicated genes are assembled by Codon Devices Inc (Cambridge, Mass.). Note that these nucleotide sequence also include DNA sequences that encode the identical or homologous polypeptides, but encompassing nucleotide substitutions to 1) alter expression levels based on E. coli codon usage tables, 2) add or remove secondary structure, 3) add or remove restriction endonuclease recognition sequences, and/or 4) facilitate gene synthesis and assembly. Alternate providers, e.g., DNA2.0 (Menlo Park, Calif.), Blue Heron Biotechnology (Bothell, Wash.), and Geneart (Regensburg, Germany), are used as noted. Sequences untenable by commercial sources may be prepared using polymerase chain reaction (PCR) from DNA or cDNA samples, or cDNA/BAC libraries. Inserts are initially propagated and sequenced in pUC19. Importantly, primary synthesis and sequence verification of each gene of interest in pUC19 provides flexibility to transfer each unit in various combinations to alternate destination vectors to drive transcription and translation of the desired enzymes. Specific and/or unique cloning sites are included at the 5′ and 3′ ends of the open reading frames (ORFs) to facilitate molecular transfers.

The required metabolic pathways are initially encoded in expression cassettes driven by constitutive promoters which are always “on.” Many such promoters are known, for example the spc ribosomal protein operon (P_{spc}) the beta-lactamase gene promoter of pBR322 (P_{bla}), the bacteriophage lambda P_{L} promoter, the replication control promoters of plasmid pBR322 (P_{RNAI} or P_{RNAII}), or the P1 or P2 promoters of the rrnB ribosomal RNA operon [Liang S T, Bipatnath M, Xu Y C, Chen S L, Dennis P, Ehrenber M, Bremer H. Activities of Constitutive Promoters in Escherichia coli. J. Mol. Biol (1999). Vol 292, Number 1, pgs 19-37]. As necessary, after designing and testing pathways, the strength of constitutive promoters are “tuned” to increase or decrease levels of transcription to optimize a network, for example, by modifying the conserved −35 and −10 elements or the spacing between these elements [Alper H, Fischer C, Nevoigt E, Stephanopoulus G. “Tuning genetic control through promoter engineering.” PNAS (2005). 102(36): 12678-12783; Jensen P R and Hammer K. “The sequence of spacers between the consensus sequences modulates the strength of prokaryotic promoters.” Appl Environ Microbiol (1998). 64(I):82-87; Mijakovic I, Petranovic D, Jensen P R. Tunable promoters in system biology. Curr Opin Biotechnol (2005). 16:329-335; De Mey M, Maertens J, Lequeux G J, Soetaert W K, Vandamme E J. “Construction and model-based analysis of a promoter library from E. coli: an indispensable tool for metabolic engineering.” BMC Biotechnology (2007) 7:34].

When constitutive expression proves non-optimal (i.e., has deleterious effects, is out of sync with the network, etc.) inducible promoters are used. Inducible promoters are “off” (not transcribed) prior to addition of an inducing agent, frequently a small molecule or metabolite. Examples of suitable inducible promoter systems include the arabinose inducible P_{bad} [Khlebnikov A, Datsenko K A, Skaug T, Wanner B L, Keasling J D. “Homogeneous expression of the P(BAD) promoter in Escherichia coli by constitutive expression of the low-affinity high-capacity AraE transporter.” Microbiology (2001). 147 (Pt 12): 3241-7], the rhamnose inducible rhaPBAD promoter [Haldimann A, Daniels L, Wanner B. J Bacteriol (1998). “Use of new methods for construction of tightly regulated arabinose and rhamnose promoter fusions in studies of the Escherichia coli phosphate regulon.” 180:1277-1286], the propionate inducible pPRO [Lee S K and Keasling J D. “A propionate-inducible expression system for enteric bacteria.” Appl Environ Microbiol (2005). 71(11):6856-62)], the IPTG-inducible lac promoter [Gronenbom. Mol Gen Genet (1976). “Overproduction of phage lambda repressor under control of the lac promoter of Escherichia coli.” 148:243-250], the synthetic tac promoter [De Boer H A, Comstock L J, Vasser M. “The tac promoter: a functional hybrid derived from the trp and lac promoters.” PNAS (1983). 80:21-25], the synthetic trc promoter [Brosius J, Erfle M, Storella J. “Spacing of the −10 and −35 regions in the tac promoter. Effect on its in vivo activity.” J Biol Chem (1985). 260:3539-3541], or the T7 RNA polymerase system [Studier F W and Moffatt B A. “Use of bacteriophage T7 RNA polymerase to direct selective high-level expression of cloned genes.” J Mol Biol (1986]. 189:113-130, the tetracycline or anhydrotetracycline-inducible tetA promoter/operator system [Skerra A. “Use of the tetracycline promoter for the tightly regulated production of a murine antibody fragment in Escherichia coli” Gene (1994). 151:131-135]. These and other naturally-occurring or synthetically-derived inducible promoters are employed (see, e.g., U.S. Pat. No. 7,235,385; Methods for enhancing expression of recombinant proteins).

Alternate origins of replication are selected to provide additional layers of expression control. The number of copies per cell contributes to the “gene dosage effect.” For example, the high copy pMB1 or colE1 origins are used to generate 300-1000 copies of each plasmid per cell, which contributes to a high level of gene expression. In contrast, plasmids encoding low copy origins, such as pSC101 or p15A, are leveraged to restrict copy number to about 1-20 copies per cell. Techniques and sequences to further modulate plasmid copy number are known (see, e.g., U.S. Pat. No. 5,565,333, Plasmid replication origin increasing the copy number of plasmid containing said origin; U.S. Pat. No. 6,806,066, Expression vectors with modified ColE1 origin of replication for control of plasmid copy number).

Expression levels are also optimized by modulation of translation efficiency. In E. coli, a Shine-Dalgarno (SD) sequence [Shine J and Dalgarno L. Nature (1975) “Determination of cistron specificity in bacterial ribosomes.” 254(5495):34-8] is a consensus sequence that directs the ribosome to the mRNA and facilitates translation initiation by aligning the ribosome with the start codon. Modulation of the SD sequence is used to increase or decrease translation efficiency as appropriate [de Boer H A, Comstock L J, Hui A, Wong E, Vasser M. Gene Amplif Anal (1983). “Portable Shine-Dalgarno regions; nucleotides between the Shine-Dalgarno sequence and the start codon effect the translation efficiency”. 3: 103-16; Mattanovich D, Weik R, Thim S, Kramer W, Bayer K, Katinger H. Ann NY Acad Sci (1996). “Optimization of recombinant gene expression in Escherichia coli.” 782:182-90.]. Of note, a high level of translation can be observed in certain contexts in the absence of an SD sequence [Xu J, Mironova R, Ivanov I G, Abouhaidar M G. J Basic Microbiol (1999). “A polylinker-derived sequence, PL, highly increased translation efficiency in Escherichia coli.” 39(1):51-60]. Secondary mRNA structure is engineered in or out of the genes of interest to modulate expression levels [Cebe R and Geiser M. Protein Expr Purif (2006). “Rapid and easy thermodynamic optimization of 5′-end of mRNA dramatically increases the level of wild type protein expression in Escherichia coli.” 45(2):374-80; Zhang W, Xiao W, Wei H, Zhang J, Tian Z. Biochem Biophys Res Commun (2006). “mRNA secondary structure at start AUG codon is a key limiting factor for human protein expression in Escherichia coli.” 349(1):69-78; Voges D, Watzele M, Nemetz C, Wizemann S, Buchberger B. Biochem Biophys Res Commun (2004). “Analyzing and enhancing mRNA translational efficiency in an Escherichia coli in vitro expression system.” 318(2):601-14]. Codon usage is also manipulated to increase or decrease levels of translation [Deng T. FEBS Lett (1997). “Bacterial expression and purification of biologically active mouse c-Fos proteins by selective codon optimization.” 409(2):269-72; Hale R S and Thompson G. Protein Expr Purif (1998). “Codon optimization of the gene encoding a domain from human type 1 neurofibromin protein results in a threefold improvement in expression level in Escherichia coli.” 12(2):185-8].

In some embodiments, each gene of interest is expressed on a unique plasmid. In preferred embodiments, the desired biosynthetic pathways are encoded on multi-cistronic plasmid vectors. A variety of commercially available plasmid systems are of use, for example pACYCDuet-1, pCDFDuet-1, pCOLADuet-1, pETDuet-1, pRSFDuet-1 from Novagen, though more useful expression vectors are designed internally and synthesized by external gene synthesis providers. When the required biosynthetic pathways necessitate DNA inserts in excess of 15 kb, cosmids, fosmids, or bacteria artificial chromosomes (BACs) are employed in lieu of plasmids.

### Genetic Manipulations

E. coli are transformed using standard techniques known to those skilled in the art, including heat shock of chemically competent cells and electroporation [Berger and Kimmel, Guide to Molecular Cloning Techniques, Methods in Enzymology volume 152 Academic Press, Inc., San Diego, Calif.; Sambrook et al. (1989) Molecular Cloning—A Laboratory Manual (2nd ed.) Vol. 1-3, Cold Spring Harbor Laboratory, Cold Spring Harbor Press, N.Y.; and Current Protocols in Molecular Biology, F. M. Ausubel et al., eds., Current Protocols, a joint venture between Greene Publishing Associates, Inc. and John Wiley & Sons, Inc., (through and including the 1997 Supplement)].

The biosynthetic pathways and modules described below are first tested and optimized using episomal plasmids described above. Non-limiting optimizations include promoter swapping and tuning, ribosome binding site manipulation, alteration of gene order (e.g., gene ABC versus BAC, CBA, CAB, BCA), co-expression of molecular chaperones, random or targeted mutagenesis of gene sequences to increase or decrease activity, folding, or allosteric regulation, expression of gene sequences from alternate species, codon manipulation, addition or removal of intracellular targeting sequences such as signal sequences, and the like.

Each gene or module is optimized individually, or alternately, in parallel. Functional promoter and gene sequences are subsequently integrated into the E. coli chromosome to enable stable propagation in the absence of selective pressure (i.e., inclusion of antibiotics) using standard techniques known to those skilled in the art.

### Disruption of Endogenous DNA Sequences

In certain instances, chromosomal DNA sequence native (i.e., “endogenous”) to the host organism are altered. Manipulations are made to non-coding regions, including promoters, ribosome binding sites, transcription terminators, and the like to increase or decrease expression of specific gene product(s). In alternate embodiments, the coding sequence of an endogenous gene is altered to affect stability, folding, activity, or localization of the intended protein. Alternately, specific genes can be entirely deleted or “knocked-out.” Techniques and methods for such manipulations are known to those skilled in the art [Datsenko K A, Wanner B L. PNAS (2000). “One-step inactivation of chromosomal genes in E. coli K-12 using PCR Products.” 97: 6640-6645; Link A J et al. J Bacteriol (1997). “Methods for generating precise deletions and insertions in the genome of wild-type Escherichia coli: Application to open reading frame characterization.” 179:6228-6237; Baba T et al. Mol Syst Biol (2006). Construction of Escherichia coli K-12 in-frame, single gene knockout mutants: the Keio collection.” 2:2006.0008; Tischer B K, von Einem J, Kaufer B, Osterrieder N. Biotechniques (2006). “Two-step red-mediated recombination for versatile high-efficiency markerless DNA manipulation in Escherichia coli.” 40(2):191-7.; McKenzie G J, Craig N L. BMC Microbiol (2006). Fast, easy and efficient: site-specific insertion of transgenes into enterobacterial chromosomes using Tn7 without need for selection of the insertion event.” 6:39].

### Selections and Assays

Selective pressure provides a valuable means for testing and optimizing the above synthetic pathways. The ability to survive in CO₂-containing minimal media under ever diminishing concentrations of exogenous organic carbon sources (i.e., glucose) provides evidence for successful implementation of a carbon fixation pathway. The ability to grow under light, but not dark, conditions confirms that modified E. coli have been rendered light-dependent. The ability to grow in the presence of CO₂, light, and minimal media confirms that the engineered organisms are photoautotrophic.

If desired, additional genetic variation can be introduced prior to selective pressure by treatment with mutagens, such as ultra-violet light, alkylators [e.g., ethyl methanesulfonate (EMS), methyl methane sulfonate (MMS), diethylsulfate (DES), and nitrosoguanidine (NTG, NG, MMG)], DNA intercalators (e.g., ethidium bromide), nitrous acid, base analogs, bromouracil, transposons, and the like.

Alternately or in addition to selective pressure, pathway activity can be monitored following growth under permissive (i.e., non-selective) conditions by measuring specific product output via various metabolic labeling studies (including radioactivity), biochemical analyses (Michaelis-Menten), gas chromatography-mass spectrometry (GC/MS), mass spectrometry, matrix assisted laser desorption ionization time-of-flight mass spectrometry (MALDI-TOF), capillary electrophoresis (CE), and high pressure liquid chromatography (HPLC).

### Other Organisms

Organisms belonging to any of the three categories of organisms listed below can be converted into a synthetophototroph and used for production of carbon-based products of interest. The first category includes preferred organisms such as Escherichia coli. The second category includes good alternative organisms such as Acetobacter aceti, Bacillus subtilis, Clostridium ljungdahlii, Clostridium thermocellum, Penicillium chrysogenum, Pichia pastoris, Saccharomyces cerevisiae, Schizosaccharomyces pombe, Pseudomonas fluorescens, and Zymomonas mobilis. The third category includes all potential heterotrophic organisms (also known as heterotrophs), typically single-celled microorganisms, but also includes cell suspensions or cultures derived from multicellular organisms.

Heterotrophic prokaryotic organisms are engineered from genera such as, but not limited to, Agrobacterium, Anaerobacter, Aquabacterium, Azorhizobium, Bacillus, Bradyrhizobium, Clostridium, Cryobacterium, Escherichia, Enterococcus, Heliobacterium, Klebsiella, Lactobacillus, Methanococcus, Methanothermobacter, Micrococcus, Mycobacterium, Oceanomonas, Pennicillium, Pseudomonas, Rhizobium, Schizochitrium, Staphylococcus, Streptococcus, Streptomyces, Thermusaquaticus, Thermaerobacter, Thermobacillus, or Zymomonas as well other bacteria noted in the “List of Prokaryotic names with Standing in Nomenclature” (LPSN) website.

A single-cell suspension culture system can be derived from multi-cellular organisms using techniques well known to those of ordinary skill in the art. Such systems and their use are included in the scope of the present invention. Exemplary multi-cellular organisms from which such single-cell suspension cultures can be derived include Spodoptera frugiperda “Sf9” cells, Drosophila melanogaster “S2” cells, and Homo sapiens Hela S3 cells.

### Fermentation Methods

The production and isolation of products from synthetophototrophic organisms can be enhanced by employing specific fermentation techniques. An essential element to maximizing production while reducing costs is increasing the percentage of the carbon source that is converted to such products. Carbon atoms, during normal cellular lifecycles, go to cellular functions including producing lipids, saccharides, proteins, and nucleic acids. Reducing the amount of carbon necessary for non-product related activities can increase the efficiency of output production. This is achieved by first growing microorganisms to a desired density. A preferred density would be that achieved at the peak of the log phase of growth. At such a point, replication checkpoint genes can be harnessed to stop the growth of cells. Specifically, quorum sensing mechanisms (reviewed in Camilli, A. and Bassler, B. L Science 311:1113; Venturi, V. FEMS Microbio Rev 30: 274; and Reading, N. C. and Sperandio, V. FEMS Microbiol Lett 254:1) can be used to activate genes such as p53, p21, or other checkpoint genes. Genes that can be activated to stop cell replication and growth in E. coli include umuDC genes, the overexpression of which stops the progression from exponential phase to stationary growth (Murli, S., Opperman, T., Smith, B. T., and Walker, G. C. 2000 Journal of Bacteriology 182: 1127.). UmuC is a DNA polymerase that can carry out translesion synthesis over non-coding lesions—the mechanistic basis of most UV and chemical mutagenesis. The umuDC gene products are required for the process of translesion synthesis and also serve as a DNA damage checkpoint. UmuDC gene products include UmuC, UmuD, umuD′, UmuD′₂C, UmuD′₂ and UmuD₂. Simultaneously, the product synthesis genes are activated, thus minimizing the need for critical replication and maintenance pathways to be used while the product is being made.

Alternatively, cell growth and product production can be achieved simultaneously. In this method, cells are grown in bioreactors with a continuous supply of inputs and continuous removal of product. Batch, fed-batch, and continuous fermentations are common and well known in the art and examples can be found in Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol (1992), 36:227.

In all production methods, inputs include carbon dioxide, water, and light. The carbon dioxide can be from the atmosphere or from concentrated sources including offgas from coal plants, refineries, cement production facilities, natural gas facilities, breweries, and others. Water can be no-salt, low-salt, marine, or high salt. Light can be solar or from artificial sources including incandescent lights, LEDs, fiber optics, and fluorescent lights.

Light-harvesting organisms are limited in their productivity to times when the solar irradiance is sufficient to activate their photosystems. In a preferred light-harvesting organism bioprocess, cells are enabled to grow and produce product with light as the energetic driver. When there is a lack of sufficient light, cells can be induced to minimize their central metabolic rate. To this end, the inducible promoters specific to product production can be heavily stimulated to drive the cell to process its energetic stores in the product of choice. With sufficient induction force, the cell will minimize its growth efforts, and use its reserves from light harvest specifically for product production. Nonetheless, net productivity is expected to be minimal during periods when sufficient light is lacking as no to few photons are net captured.

In a preferred embodiment, the cell is engineered such that the final product is released from the cell. In embodiments where the final product is released from the cell, a continuous process can be employed. In this approach, a reactor with organisms producing desirable products can be assembled in multiple ways. In one embodiment, the reactor is operated in bulk continuously, with a portion of media removed and held in a less agitated environment such that an aqueous product will self-separate out with the product removed and the remainder returned to the fermentation chamber. In embodiments where the product does not separate into an aqueous phase, media is removed and appropriate separation techniques (e.g., chromatography, distillation, etc.) are employed.

In an alternate embodiment, the product is not secreted by the cells. In this embodiment, a batch-fed fermentation approach is employed. In such cases, cells are grown under continued exposure to inputs (light, water, and carbon dioxide) as specified above until the reaction chamber is saturated with cells and product. A significant portion to the entirety of the culture is removed, the cells are lysed, and the products are isolated by appropriate separation techniques (e.g., chromatography, distillation, filtration, centrifugation, etc.).

In a preferred embodiment, the fermentation chamber will enclose a fermentation that is undergoing a continuous reductive fermentation. In this instance, a stable reductive environment is created. The electron balance is maintained by the release of carbon dioxide (in gaseous form). Augmenting the NAD/H and NADP/H balance, as described above, also can be helpful for stabilizing the electron balance.

### Detection and Analysis of Gene and Cell Products

Any of the standard analytical methods, such as gas chromatography-mass spectrometry, and liquid chromatography-mass spectrometry, HPLC, capillary electrophoresis, Matrix-Assisted Laser Desorption Ionization time of flight-mass spectrometry, etc., can be used to analyze the levels and the identity of the product produced by the modified organisms of the present invention.

The ability to detect formation of a new, functional biochemical pathway in the synthetophototrophic cell is important to the practice of the subject methods. In general, the assays are carried out to detect heterologous biochemical transformation reactions of the host cell that produce, for example, small organic molecules and the like as part of a de novo synthesis pathway, or by chemical modification of molecules ectopically provided in the host cell's environment. The generation of such molecules by the host cell can be detected in “test extracts,” which can be conditioned media, cell lysates, cell membranes, or semi-purified or purified fractionation products thereof. The latter can be, as described above, prepared by classical fractionation/purification techniques, including phase separation, chromatographic separation, or solvent fractionation (e.g., methanol ethanol, acetone, ethyl acetate, tetrahydrofuran (THF), acetonitrile, benzene, ether, bicarbonate salts, dichloromethane, chloroform, petroleum ether, hexane, cyclohexane, diethyl ether and the like). Where the assay is set up with a responder cell to test the effect of an activity produced by the host cell on a whole cell rather than a cell fragment, the host cell and test cell can be co-cultured together (optionally separated by a culture insert, e.g. Collaborative Biomedical Products, Bedford, Mass., Catalog #40446).

In certain embodiments, the assay is set up to directly detect, by chemical or photometric techniques, a molecular species which is produced (or destroyed) by a biosynthetic pathway of the recombinant host cell. Such a molecular species' production or degradation must be dependent, at least in part, on expression of the heterologous genomic DNA. In other embodiments, the detection step of the subject method involves characterization of fractionated media/cell lysates (the test extract), or application of the test extract to a biochemical or biological detection system. In other embodiments, the assay indirectly detects the formation of products of a heterologous pathway by observing a phenotypic change in the host cell, e.g. in an autocrine fashion, which is dependent on the establishment of a heterologous biosynthetic pathway in the host cell.

In certain embodiments, analogs related to a known class of compounds are sought, as for example analogs of alkaloids, aminoglycosides, ansamacrolides, beta-lactams (including penicillins and cephalosporins), carbapenems, terpinoids, prostanoid hormones, sugars, fatty acids, lincosaminides, macrolides, nitrofurans, nucleosides, oligosaccharides, oxazolidinones, peptides and polypeptides, phenazines, polyenes, polyethers, quinolones, tetracyclines, streptogramins, sulfonamides, steroids, vitamins and xanthines. In such embodiments, if there is an available assay for directly identifying and/or isolating the natural product, and it is expected that the analogs would behave similarly under those conditions, the detection step of the subject method can be as straightforward as directly detecting analogs of interest in the cell culture media or preparation of the cell. For instance, chromatographic or other biochemical separation of a test extract may be carried out, and the presence or absence of an analog detected, e.g., spectrophotometrically, in the fraction in which the known compounds would occur under similar conditions. In certain embodiments, such compounds can have a characteristic fluorescence or phosphorescence which can be detected without any need to fractionate the media and/or recombinant cell.

In related embodiments, whole or fractionated culture media or lysate from a recombinant host cell can be assayed by contacting the test sample with a heterologous cell (“test cell”) or components thereof. For instance, a test cell, which can be prokaryotic or eukaryotic, is contacted with conditioned media (whole or fractionated) from a recombinant host cell, and the ability of the conditioned media to induce a biological or biochemical response from the test cell is assessed. For instance, the assay can detect a phenotypic change in the test cell, as for example a change in: the transcriptional or translational rate or splicing pattern of a gene; the stability of a protein; the phosphorylation, prenylation, methylation, glycosylation or other post translational modification of a protein, nucleic acid or lipid; the production of 2nd messengers, such as cAMP, inositol phosphates and the like. Such effects can be measured directly, e.g., by isolating and studying a particular component of the cell, or indirectly such as by reporter gene expression, detection of phenotypic markers, and cytotoxic or cytostatic activity on the test cell.

When screening for bioactivity of test compounds produced by the recombinant host cells, intracellular second messenger generation can be measured directly. A variety of intracellular effectors have been identified. For instance, for screens intended to isolate compounds, or the genes which encode the compounds, as being inhibitors or potentiators of receptor- or ion channel-regulated events, the level of second messenger production can be detected from downstream signaling proteins, such as adenylyl cyclase, phosphodiesterases, phosphoinositidases, phosphoinositol kinases, and phospholipases, as can the intracellular levels of a variety of ions.

In still other embodiments, the detectable signal can be produced by use of enzymes or chromogenic/fluorescent probes whose activities are dependent on the concentration of a second messenger, e.g., such as calcium, hydrolysis products of inositol phosphate, cAMP, etc.

Many reporter genes and transcriptional regulatory elements are known to those of skill in the art and others may be identified or synthesized by methods known to those of skill in the art. Examples of reporter genes include, but are not limited to CAT (chloramphenicol acetyl transferase) (Alton and Vapnek (1979), Nature 282: 864-869) luciferase, and other enzyme detection systems, such as beta-galactosidase; firefly luciferase (deWet et al. (1987), Mol. Cell. Biol. 7:725-737); bacterial luciferase (Engebrecht and Silverman (1984), PNAS 1: 4154-4158; Baldwin et al. (1984), Biochemistry 23: 3663-3667); alkaline phosphatase (Toh et al. (1989) Eur. J. Biochem. 182: 231-238, Hall et al. (1983) J. Mol. Appl. Gen. 2: 101), human placental secreted alkaline phosphatase (Cullen and Malim (1992) Methods in Enzymol. 216:362-368); β-lactamase or GST.

Transcriptional control elements for use in the reporter gene constructs, or for modifying the genomic locus of an indicator gene include, but are not limited to, promoters, enhancers, and repressor and activator binding sites. Suitable transcriptional regulatory elements may be derived from the transcriptional regulatory regions of genes whose expression is rapidly induced, generally within minutes, of contact between the cell surface protein and the effector protein that modulates the activity of the cell surface protein. Examples of such genes include, but are not limited to, the immediate early genes (see, Sheng et al. (1990) Neuron 4: 477-485), such as c-fos. Immediate early genes are genes that are rapidly induced upon binding of a ligand to a cell surface protein. The transcriptional control elements that are preferred for use in the gene constructs include transcriptional control elements from immediate early genes, elements derived from other genes that exhibit some or all of the characteristics of the immediate early genes, or synthetic elements that are constructed such that genes in operative linkage therewith exhibit such characteristics. The characteristics of preferred genes from which the transcriptional control elements are derived include, but are not limited to, low or undetectable expression in quiescent cells, rapid induction at the transcriptional level within minutes of extracellular simulation, induction that is transient and independent of new protein synthesis, subsequent shut-off of transcription requires new protein synthesis, and mRNAs transcribed from these genes have a short half-life. It is not necessary for all of these properties to be present.

In still other embodiments, the detection step is provided in the form of a cell-free system, e.g., a cell-lysate or purified or semi-purified protein or nucleic acid preparation. The samples obtained from the recombinant host cells can be tested for such activities as inhibiting or potentiating such pairwise complexes (the “target complex”) as involving protein-protein interactions, protein-nucleic acid interactions, protein-ligand interactions, nucleic acid-nucleic acid interactions, and the like. The assay can detect the gain or loss of the target complexes, e.g. by endogenous or heterologous activities associated with one or both molecules of the complex.

Assays that are performed in cell-free systems, such as may be derived with purified or semi-purified proteins, are often preferred as “primary” screens in that they can be generated to permit rapid development and relatively easy detection of an alteration in a molecular target when contacted with a test sample. Moreover, the effects of cellular toxicity and/or bioavailability of the test sample can be generally ignored in the in vitro system, the assay instead being focused primarily on the effect of the sample on the molecular target as may be manifest in an alteration of binding affinity with other molecules or changes in enzymatic properties (if applicable) of the molecular target. Detection and quantification of the pairwise complexes provides a means for determining the test samples efficacy at inhibiting (or potentiating) formation of complexes. The efficacy of the compound can be assessed by generating dose response curves from data obtained using various concentrations of the test sample. Moreover, a control assay can also be performed to provide a baseline for comparison. For instance, in the control assay conditioned media from untransformed host cells can be added.

The amount of target complex may be detected by a variety of techniques. For instance, modulation in the formation of complexes can be quantitated using, for example, detectably labeled proteins or the like (e.g., radiolabeled, fluorescently labeled, or enzymatically labeled), by immunoassay, or by chromatographic detection.

In still other embodiments, a purified or semi-purified enzyme can be used to assay the test samples. The ability of a test sample to inhibit or potentiate the activity of the enzyme can be conveniently detected by following the rate of conversion of a substrate for the enzyme.

In yet other embodiments, the detection step can be designed to detect a phenotypic change in the host cell which is induced by products of the expression of the heterologous genomic sequences. Many of the above-mentioned cell-based assay formats can also be used in the host cell, e.g., in an autocrine-like fashion.

In addition to providing a basis for isolating biologically-active molecules produced by the recombinant host cells, the detection step can also be used to identify genomic clones which include genes encoding biosynthetic pathways of interest. Moreover, by iterative and/or combinatorial sub-cloning methods relying on such detection steps, the individual genes which confer the detected pathway can be cloned from the larger genomic fragment.

The subject screening methods can be carried in a differential format, e.g. comparing the efficacy of a test sample in a detection assay derived with human components with those derived from, e.g., fungal or bacterial components. Thus, selectivity as a bacteriocide or fungicide can be a criterion in the selection protocol.

The host strain need not produce high levels of the novel compounds for the method to be successful. Expression of the genes may not be optimal, global regulatory factors may not be present, or metabolite pools may not support maximum production of the product. The ability to detect the metabolite will often not require maximal levels of production, particularly when the bioassay is sensitive to small amounts of natural products. Thus initial submaximal production of compounds need not be a limitation to the success of the subject method.

Finally, as indicated above, the test sample can be derived from, for example, conditioned media or cell lysates. With regard to the latter, it is anticipated that in certain instances there may be heterologously-expressed compounds that may not be properly exported from the host cell. There are a variety of techniques available in the art for lysing cells. A preferred approach is another aspect of the present invention, namely, the use of a host cell-specific lysis agent. For instance phage (e.g., P1, λ, φ80) can be used to selectively lyse E coli. Addition of such phage to grown cultures of E. coli host cells can maximize access to the heterologous products of new biosynthetic pathways in the cell. Moreover, such agents do not interfere with the growth of a tester organism, e.g., a human cell, that may be co-cultured with the host cell library.

### Metabolic Optimization

As part of the optimization process, the invention also provides steps to eliminate undesirable side reactions, if any, that may consume carbon and energy but do not produce useful products (such as hydrocarbons, wax esters, surfactants and other hydrocarbon products). These steps may be helpful in that they can help to improve yields of the desired products.

A combination of different approaches may be used. Such approaches include, for example, metabolomics (which may be used to identify undesirable products and metabolic intermediates that accumulate inside the cell), metabolic modeling and isotopic labeling (for determining the flux through metabolic reactions contributing to hydrocarbon production), and conventional genetic techniques (for eliminating or substantially disabling unwanted metabolic reactions). For example, metabolic modeling provides a means to quantify fluxes through the cell's metabolic pathways and determine the effect of elimination of key metabolic steps. In addition, metabolomics and metabolic modeling enable better understanding of the effect of eliminating key metabolic steps on production of desired products.

To predict how a particular manipulation of metabolism affects cellular metabolism and synthesis of the desired product, a theoretical framework was developed to describe the molar fluxes through all of the known metabolic pathways of the cell. Several important aspects of this theoretical framework include: (i) a relatively complete database of known pathways in Escherichia coli, (ii) incorporation of the growth-rate dependence of cell composition and energy requirements, (iii) experimental measurements of the amino acid composition of proteins and the fatty acid composition of membranes at different growth rates and dilution rates and (iv) experimental measurements of side reactions which are known to occur as a result of metabolism manipulation. These new developments allow significantly more accurate prediction of fluxes in key metabolic pathways and regulation of enzyme activity. (Keasling, J. D. et al., “New tools for metabolic engineering of Escherichia coli,” In Metabolic Engineering, Publisher Marcel Dekker, New York, Nym 1999; Keasling, J. D, “Gene-expression tools for the metabolic engineering of bacteria,” Trends in Biotechnology, 17, 452-460, 1999; Martin, V. J. J., et al., “Redesigning cells for production of complex organic molecules,” ASM News 68, 336-343 2002; Henry, C. S., et al., “Genome-Scale Thermodynamic Analysis of Escherichia coli Metabolism,” Biophys. J., 90, 1453-1461, 2006.)

Such types of models have been applied, for example, to analyze metabolic fluxes in organisms responsible for enhanced biological phosphorus removal in wastewater treatment reactors and in filamentous fungi producing polyketides. See, for example, Pramanik, et al., “A stoichiometric model of Escherichia coli metabolism: incorporation of growth-rate dependent biomass composition and mechanistic energy requirements.” Biotechnol. Bioeng. 56, 398-421, 1997; Pramanik, et al., “Effect of carbon source and growth rate on biomass composition and metabolic flux predictions of a stoichiometric model.” Biotechnol. Bioeng. 60, 230-238, 1998; Pramanik et al., “A flux-based stoichiometric model of enhanced biological phosphorus removal metabolism.” Wat. Sci. Tech. 37, 609-613, 1998; Pramanik et al., “Development and validation of a flux-based stoichiometric model for enhanced biological phosphorus removal metabolism.” Water Res. 33, 462-476, 1998.

### Products

The recombinant microorganisms of the present invention may be engineered to yield products categories, including but not limited to, biological sugars, hydrocarbon products, solid forms, and pharmaceuticals.

Biological sugars include but are not limited to glucose, starch, cellulose, hemicellulose, glycogen, xylose, dextrose, fructose, lactose, fructose, galactose, uronic acid, maltose, and polyketides. In preferred embodiments, the biological sugar may be glycogen, starch, or cellulose.

Cellulose is the most abundant form of living terrestrial biomass (Crawford, R. L. 1981. Lignin biodegradation and transformation, John Wiley and Sons, New York.). Cellulose, especially cotton linters, is used in the manufacture of nitrocellulose. Cellulose is also the major constituent of paper. Cellulose monomers (beta-glucose) are linked together through 1,4 glycosidic bonds. Cellulose is a straight chain (no coiling occurs). In microfibrils, the multiple hydroxide groups hydrogen-bond with each other, holding the chains firmly together and contributing to their high tensile strength. Given a cellulose material, the portion that does not dissolve in a 17.5% solution of sodium hydroxide at 20° C. is Alpha cellulose, which is true cellulose; the portion that dissolves and then precipitates upon acidification is Beta cellulose, and the proportion that dissolves but does not precipitate is Gamma cellulose. Hemicellulose is a class of plant cell-wall polysaccharide that can be any of several heteropolymers. These include xylane, xyloglucan, arabinoxylan, arabinogalactan, glucuronoxylan, glucomannan, and galactomannan. This class of polysaccharides is found in almost all cell walls along with cellulose. Hemicellulose is lower in weight than cellulose, and cannot be extracted by hot water or chelating agents, but can be extracted by aqueous alkali. Polymeric chains bind pectin and cellulose, forming a network of cross-linked fibers.

There are essentially three types of hydrocarbon products: (1) aromatic hydrocarbon products, which have at least one aromatic ring; (2) saturated hydrocarbon products, which lack double, triple or aromatic bonds; and (3) unsaturated hydrocarbon products, which have one or more double or triple bonds between carbon atoms. A “hydrocarbon product” may be further defined as a chemical compound that consists of C, H, and optionally O, with a carbon backbone and atoms of hydrogen and oxygen, attached to it. Oxygen may be singly or double bonded to the backbone and may be bound by hydrogen. In the case of ethers and esters, oxygen may be incorporated into the backbone, and linked by two single bonds, to carbon chains. A single carbon atom may be attached to one or more oxygen atoms. Hydrocarbon products may also include the above compounds attached to biological agents including proteins, coenzyme A and acetyl coenzyme A. Hydrocarbon products include, but are not limited to, hydrocarbons, alcohols, aldehydes, carboxylic acids, ethers, esters, carotenoids, and ketones.

Hydrocarbon products also include alkanes, alkenes, alkynes, dienes, isoprenes, alcohols, aldehydes, carboxylic acids, surfactants, wax esters, polymeric chemicals [polyphthalate carbonate (PPC), polyester carbonate (PEC), polyethylene, polypropylene, polystyrene, polyhydroxyalkanoates (PHAs), poly-beta-hydroxybutryate (PHB), polylactide (PLA), and polycaprolactone (PCL)], monomeric chemicals [propylene glycol, ethylene glycol, and 1,3-propanediol, ethylene, acetic acid, butyric acid, 3-hydroxypropanoic acid (3-HPA), acrylic acid, and malonic acid], and combinations thereof. In some preferred embodiments, the hydrocarbon products are alkanes, alcohols, surfactants, wax esters and combinations thereof. Other hydrocarbon products include fatty acids, acetyl-CoA bound hydrocarbons, acetyl-CoA bound carbohydrates, and polyketide intermediates.

Recombinant microorganisms can be engineered to produce hydrocarbon products and intermediates over a large range of sizes. Specific alkanes that can be produced include, for example, ethane, propane, butane, pentane, hexane, heptane, octane, nonane, decane, undecane, dodecane, tridecane, tetradecane, pentadecane, hexadecane, heptadecane, and octadecane. In preferred embodiments, the hydrocarbon products are octane, decane, dodecane, tetradecane, and hexadecane. Hydrocarbon precursors such as alcohols that can be produced include, for example, ethanol, propanol, butanol, pentanol, hexanol, heptanol, octanol, nonanol, decanol, undecanol, dodecanol, tridecanol, tetradecanol, pentadecanol, hexadecanol, heptadecanol, and octadecanol. In more preferred embodiments, the alcohol is selected from ethanol, propanol, butanol, pentanol, hexanol, heptanol, octanol, nonanol, and decanol.

Surfactants are used in a variety of products, including detergents and cleaners, and are also used as auxiliaries for textiles, leather and paper, in chemical processes, in cosmetics and pharmaceuticals, in the food industry and in agriculture. In addition, they may be used to aid in the extraction and isolation of crude oils which are found hard to access environments or as water emulsions. There are four types of surfactants characterized by varying uses. Anionic surfactants have detergent-like activity and are generally used for cleaning applications. Cationic surfactants contain long chain hydrocarbons and are often used to treat proteins and synthetic polymers or are components of fabric softeners and hair conditioners. Amphoteric surfactants also contain long chain hydrocarbons and are typically used in shampoos. Non-ionic surfactants are generally used in cleaning products.

Hydrocarbons can additionally be produced as biofuels. A biofuel is any fuel that derives from a biological source—recently living organisms or their metabolic byproducts, such as manure from cows. A biofuel may be further defined as a fuel derived from a metabolic product of a living organism. Preferred biofuels include, but are not limited to, biodiesel, biocrude, ethanol, “renewable petroleum,” butanol, and propane.

Solid forms of carbon including, for example, coal, graphite, graphene, cement, carbon nanotubes, carbon black, diamonds, and pearls. Pure carbon solids such as coal and diamond are the preferred solid forms.

Pharmaceuticals can be produced including, for example, isoprenoid-based taxol and artemisinin, or oseltamivir.

### Proteorhodopsin Photosystem

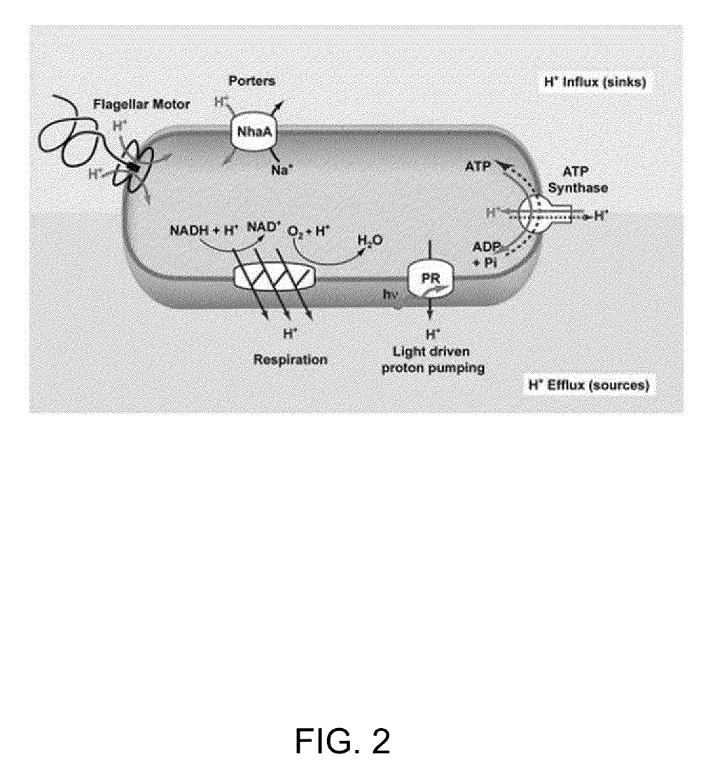

The genes of proteorhodopsin photosystems have been shown previously to be naturally linked genes from a wild type host. For example, a gene encoding proteorhodopsin and a set of genes for retinal biosynthesis have been identified from the uncultured marine bacterium HF10_{—}19p19 (accession number EF100190) SEQ ID NOS 162, 156, 151, 143, 136, 130 and 123; and HF10_{—}25f10 (accession number EF100190) SEQ ID NOS 163, 157, 152, 144, 137, 129 and 124 (Martinez, A., et al., PNAS USA, vol. 104:13 (2007) 5590-5595). Other uncultured marine bacteria having a linked set of genes for a proteorhodopsin photosystem include BAC17H8, SEQ ID NOS 165, 159, 154, 146, 139, 132 and 126 (accession number DQ068068; Futterer, O., et al., PNAS USA, vol. 101:24 (2004) 9091-9096); and BAC46A06 SEQ ID NOS 164, 158, 153, 145, 138, 131 and 125 (accession number DQ088847; Sabehi, G., et al., PLoS Biol vol 3:8 (2005) e273), also have been identified as hosts carrying a set of naturally linked genes for proteorhodopsin and retinal biosynthesis. Additionally, light capture via a light-driven proton pump, such as proteorhodopsin has been previously shown to generate a proton motive force that turns the flagellar motor in E. coli (FIG. 2).

Certain aspects of the invention include genes encoding the proteorhodopsin photosystem that have been codon and expression optimized as set forth in SEQ ID NOS 182, 194, 204, 220, 234, 246, 260; in SEQ ID NOS 180, 192, 202, 218, 232, 248, 258; in SEQ ID NOS 176, 188, 198, 214, 228, 242, 254; and SEQ ID NOS 178, 190, 200, 216, 230, 244 and 256, which can be introduced into a host cell as individual gene constructs or as a single synthetic operon. In one embodiment, the synthetic operon can be introduced into a heterologous bacterial host cell including, but not limited to, E. coli, as a functional, heterologous proteorhodopsin photosystem.

In certain embodiments a proteorhodopsin photosystem comprising a bacteriorhodopsin proton pump and retinal biosynthetic genes are selected from thermophilic hosts and combined into a single, synthetic operon or expressed as individual gene constructs. It will be understood that “proteorhodopsin” and “bacteriorhodopsin” are interchangeable with respect to functioning as a light-activated proton pump as used for the present invention.

A combination of proteorhodopsin photosystem genetic elements from host cells thriving in high temperature environments genetically engineered into heterologous host cells is advantageous for use in the elevated temperature environments such as bioreactors. For example, Picrophilis torridus (P. torridus; accession number NC_{—}005877) have the following genes representing an isopentenyl-diphosphate delta-isomerase SEQ ID NO:166, a carotene hydroxylase SEQ ID NO:160, a lycopene cyclase SEQ ID NO: 155, a phytoene dehydrogenase SEQ ID NO: 149, a phytoene synthase SEQ ID NO:141, and a geranylgeranyl pyrophosphate synthetase SEQ ID NO:135. In Thermosynechococcus elongotus BP-1 (T. elongotus; accession number NC_{—}004113) are genes representing a phytoene dehydrogenase SEQ ID NO: 148, a phytoene synthase SEQ ID NO:140, and a geranylgeranyl pyrophosphate synthetase SEQ ID NO:134. In Salinibacter ruber (S. ruber; accession number NC_{—}007677) are genes representing an isopentenyl-diphosphate delta-isomerase SEQ ID NO:168, a 15,15′-beta carotene dioxygenase SEQ ID NO:161, a phytoene dehydrogenase SEQ ID NO:150, a phytoene synthase SEQ ID NO: 142, and a bacteriorhodopsin SEQ ID NO: 128. In Pyrobaculum arsenaticum (P. arsenaticum; accession number NC_{—}009376) are genes representing a phytoene dehydrogenase SEQ ID NO: 147, isopentenyl-diphosphate delta-isomerase SEQ ID NO:167, and a geranylgeranyl pyrophosphate synthetase SEQ ID NO:133.

The above genes from P. torridus, T. elongotus, S. ruber and P. arsenaticum encoding photosystem genetic elements have been codon and expression optimized in the present invention SEQ ID NOS 174, 186, 196, 208, 224, 236; SEQ ID NOS 210, 226, 238; SEQ ID NOS 170, 184, 206, 222, 250; and SEQ ID NOS 172, 212 and 240, and can be expressed individually in a host cell or as a complete synthetic operon encoding a heterologous proteorhodopsin photosystem. In a preferred embodiment, the synthetic operon can be introduced into yeast host cells including Saccharomyces cerevisiae or Pichia pastoris, filamentous fungi host cells including Aspergillus, Trichoderma and Neurospora, mammalian host cells including murine and human, or insect host cells, and the like, as a heterologous, functional proteorhodopsin photosystem.

In certain aspects of the invention, expressing rational combinations of individual genetic elements found in a variety of cell types can result in a functional proteorhodopsin photosystem. For example, the genes for synthetic photoexpression operons can be a combination of genes from extremophile cells and/or non-extremophile cells. In one embodiment, an incomplete set of natural or codon and expression optimized genetic elements for a proteorhodopsin photosystem of P. torridus comprising an isopentenyl-diphosphate delta-isomerase, a carotene hydroxylase, a lycopene cyclase, a phytoene dehydrogenase, a phytoene synthase and a geranylgeranyl pyrophosphate synthetase may be genetically engineered into a host cell in combination with a proteorhodopsin natural or codon and expression optimized gene of the uncultured marine bacterium HF_{—}25F-10 or a bacteriodopsin gene of Candidatus pelagibacter ubique HTCC1062 (accession number NC_{—}007205; natural SEQ ID NO:127; optimized SEQ ID NO:252) to form a complete, functional proteorhodopsin photosystem. Alternatively, genetic elements for a complete photosystem from unrelated host cells may be combined to form a complete, functional proteorhodopsin photosystem for the specific host cell and specific environment such as a bioreactor operating at higher than ambient temperatures. In a preferred embodiment, genes represented by an isopentenyl-diphosphate delta-isomerase, a geranylgeranyl pyrophosphate synthetase and a lycopene cyclase gene from a P. torridus cell may be combined with a 15,15′-beta carotene dioxygenase, a phytoene dehydrogenase, a phytoene synthase, and a bacteriorhodopsin gene represented in a thermophilic S. ruber cell to form a fully functional proteorhodopsin photosystem for high temperature environments.

In yet another embodiment, a rational combination of genes from unrelated cells may be combined to form a functional proteorhodopsin photosystem wherein the production of ATP is in excess of the pool of ATP produced from a natural set of linked genes introduced into a heterologous host cell. Preferably, the rational combination of genes comprising a functional photosystem will be comprised of genes from thermophilic cells that result in higher ATP energy reserves than provided by a set of naturally linked, non-thermophilic cells when active in a high temperature bioreactor environment.

In another preferred embodiment, genes from unrelated heterologous cells combined to form a functional proteorhodopsin photosystem can produce pools of ATP in excess of endogenous host cell levels. Preferably, the rational combination of genes comprising a functional photosystem will be comprised of genes from thermophilic cells that result in higher ATP energy reserves than provided by alternative, endogenous biochemical pathways of a host cell.

In a more preferred embodiment, genes from unrelated heterologous cells combined to form a functional proteorhodopsin photosystem will produce pools of ATP to provide an additional or alternative ATP energy resource for the production of biofuels or other carbon based products of interest.

In an even more preferred embodiment, genes from unrelated heterologous cells combined to form a functional proteorhodopsin photosystem will produce pools of ATP in excess of endogenous host cell levels or in excess of a photosystem encoded by a set of linked genes to provide an additional or alternative ATP energy resource for the production of biofuels or other carbon based products of interest.

A preferred embodiment for the present invention is a method for genetically engineering into a host cell a photon activated proton pump comprising selecting from a first cell at least one nucleotide sequence from the group encoding polypeptides for proteorhodopsin, isopentenyl diphosphate δ-isomerase, geranylgeranyl pyrophosphate synthase, phytoene dehydrogenase, phytoene synthase, lycopene cyclase and carotene dehydrogenase; selecting from at least one second cell nucleotide sequences from the group encoding polypeptides for proteorhodopsin, isopentenyl diphosphate δ-isomerase, geranylgeranyl pyrophosphate synthase, phytoene dehydrogenase, phytoene synthase, lycopene cyclase and carotene dehydrogenase; combining said nucleotide sequences into a nucleic acid construct encoding a functional proteorhodopsin photosystem; and introducing into the host cell said nucleic acid construct.

Another preferred embodiment for the present invention is a method for genetically engineering into a host cell a photon activated proton pump wherein the nucleotide sequences of a nucleic acid construct encoding genes for the photon activated proton pump are modified for host specific codon usage and gene expression control.

Another preferred embodiment for the present invention is a method for genetically engineering into a host cell a photon activated proton pump wherein the nucleotide sequences of a nucleic acid construct encoding genes for the photon activated proton pump are modified for host specific codon usage and gene expression control and increase the synthesis of adenosine triphosphate in excess of endogenous adenosine triphosphate levels.

Another preferred embodiment for the present invention is a method for genetically engineering into a host cell a photon activated proton pump wherein the nucleotide sequences of a nucleic acid construct encoding genes for the photon activated proton pump are modified for host specific codon usage and gene expression control and increase the synthesis of adenosine triphosphate in excess of a proteorhodopsin photosystem introduced to the cell as a set of natural linked genes from a single cell.

In a more preferred embodiment, genes from unrelated heterologous cells combined to form a functional proteorhodopsin photosystem will produce pools of ATP to provide an additional or alternative ATP energy resource for the production of biofuels or other carbon based products of interest.

In an even more preferred embodiment, genes from unrelated heterologous cells combined to form a functional proteorhodopsin photosystem will produce pools of ATP in excess of endogenous host cell levels or in excess of a photosystem encoded by a set of linked genes to provide an additional or alternative ATP energy resource for the production of biofuels or other carbon based products of interest.

A preferred embodiment for the present invention is a method for genetically engineering into a host cell a photon activated proton pump comprising selecting from a first cell at least one nucleotide sequence from the group encoding polypeptides for proteorhodopsin, isopentenyl diphosphate δ-isomerase, geranylgeranyl pyrophosphate synthase, phytoene dehydrogenase, phytoene synthase, lycopene cyclase and carotene dehydrogenase; selecting from at least one second cell nucleotide sequences from the group encoding polypeptides for proteorhodopsin, isopentenyl diphosphate δ-isomerase, geranylgeranyl pyrophosphate synthase, phytoene dehydrogenase, phytoene synthase, lycopene cyclase and carotene dehydrogenase; combining said nucleotide sequences into a nucleic acid construct encoding a functional proteorhodopsin photosystem; and introducing into the host cell said nucleic acid construct.

Another preferred embodiment for the present invention is a method for genetically engineering into a host cell a photon activated proton pump wherein the nucleotide sequences of a nucleic acid construct encoding genes for the photon activated proton pump are modified for host specific codon usage and gene expression control.

Another preferred embodiment for the present invention is a method for genetically engineering into a host cell a photon activated proton pump wherein the nucleotide sequences of a nucleic acid construct encoding genes for the photon activated proton pump are modified for host specific codon usage and gene expression control and increase the synthesis of adenosine triphosphate in excess of endogenous adenosine triphosphate levels.

Another preferred embodiment for the present invention is a method for genetically engineering into a host cell a photon activated proton pump wherein the nucleotide sequences of a nucleic acid construct encoding genes for the photon activated proton pump are modified for host specific codon usage and gene expression control and increase the synthesis of adenosine triphosphate in excess of a proteorhodopsin photosystem introduced to the cell as a set of natural linked genes from a single cell.

Another preferred embodiment for the present invention is a method for genetically engineering into a host cell a photon activated proton pump wherein the nucleotide sequences of a nucleic acid construct encoding genes for the photon activated proton pump are modified for host-specific codon usage and gene expression control wherein the selected nucleotide sequences are from extremophile host cells including, but not limited to, Aquifex aeolicus, Bacillus halodurans, Bacillus stearothermophilus, Carboxydothermus hydrogenoformans Z-2901, Chloroflexus aurantiacus, Desulfotalea psychrophila LSv54, Deinococcus radiodurans, Salinibacter ruber DSM 13855, Thermoanaerobacter tengcongensis, Thermobifida fusca YX, Thermotoga maritime, Thermus thermophilus HB27, Thermus thermophilus HB8, Thermus aquaticus, Thermosynechococcus elongates, Thermococcus litoralis, Aeropyrum pernix, Geothermobacterium ferrireducens, Hyperthermus butylicus, Ignicoccus hospitalis, Staphylothermus marinus, Metallosphaera sedula, Sulfolobus acidocaldarius, Sulfolobus solfataricus, Sulfolobus tokodaii, Synechococcus lividis, Caldivirga maquilingensis, Pyrolobus fumarii, Pyrobaculum aerophilum, Pyrobaculum arsenaticum, Pyrobaculum calidifontis, Pyrobaculum islandicum, Thermofilum pendens, Thermoproteus neutrophilus, Pyrococcus abyssi, Pyrococcus furiosus, Pyrococcus horikoshii, Picrophilus torridus, Pyrodictium abyssi, Thermoplasma acidophilum, Thermoplasma volcanium, Methanobacterium thermoautotrophicum, Methanocaldococcus jannaschii, and Methanopyrus kandleri.

A more preferred embodiment for the present invention is a method for producing carbon based products of interest comprising selecting from a first cell at least one nucleotide sequence from the group encoding polypeptides for proteorhodopsin, isopentenyl diphosphate δ-isomerase, geranylgeranyl pyrophosphate synthase, phytoene dehydrogenase, phytoene synthase, lycopene cyclase and carotene dehydrogenase; selecting from at least one second cell nucleotide sequences from the group encoding polypeptides for proteorhodopsin, isopentenyl diphosphate δ-isomerase, geranylgeranyl pyrophosphate synthase, phytoene dehydrogenase, phytoene synthase, lycopene cyclase and carotene dehydrogenase; combining said nucleotide sequences into a nucleic acid construct encoding a functional proteorhodopsin photosystem; introducing into the host cell said nucleic acid construct; culturing the host cell to produce carbon based biofuels or products of interest. The carbon-based products of interest are removed from said host cell.

Another more preferred embodiment for the present invention is a method for producing carbon based products of interest genetically engineering into a host cell a photon activated proton pump wherein the nucleotide sequences of said nucleic acid construct are modified for host-specific codon usage and gene expression control.

Another more preferred embodiment for the present invention is a method for producing carbon based products of interest by genetically engineering into a host cell a photon activated proton pump wherein the nucleotide sequences of a nucleic acid construct encoding genes for the photon activated proton pump are modified for host specific codon usage and gene expression control and increase the synthesis of adenosine triphosphate in excess of endogenous adenosine triphosphate levels.

Another more preferred embodiment for the present invention is a method for producing carbon based products of interest by genetically engineering into a host cell a photon activated proton pump wherein the nucleotide sequences of a nucleic acid construct encoding genes for the photon activated proton pump are modified for host specific codon usage and gene expression control and increase the synthesis of adenosine triphosphate in excess of a proteorhodopsin photosystem introduced to the cell as a set of natural linked genes from a single cell.

In another aspect, the proteins of a heterologous proteorhodopsin photosystem described herein can be engineered to have peptide signal sequences localizing the expressed gene product to the host cell outer membrane. Signal peptides have been shown to be important for localization to cellular compartments such as a thylakoid lumen, the host cell outer membrane, plasma membrane or the periplasmic space (Rajalahti, T., et al., J. Proteome Res. Vol 6 (2007) 2420-2434). In a preferred embodiment, signal peptides specific for an outer membrane can be engineered into the nucleotide coding sequence to increase the efficacy of cellular localization of proteorhodopsin to a host cell outer membrane. For example, certain peptide signal sequences of Synechocystis sp PCC6803 are known to target the outer membrane (Rajalahti, T., et al.; included herein by reference in its entirety). In another example, retinal biosynthesis genes can be combined with nucleotide sequences for peptide signal sequences targeting the periplasmic space. Peptide signal sequences from Synechocystis sp PCC6803 are known to target the periplasmic space (Rajalahti, T., et al.; included herein by reference in its entirety).

In one embodiment, gene sequences for a functional photosystem can be designed to have heterologous sequences for signal peptides to target the expressed photosystem gene products to the appropriate region of the host cell. In a preferred embodiment, heterologous photosystem genes that are codon and expression optimized for an E. coli host cell will incorporate a codon and expression optimized signal sequence from a Synechocystis sp. PCC6803 cell to target the expressed gene product to the appropriate region of the host cell. In yet another embodiment, the synthetic operons of the invention described herein will incorporate a codon and expression optimized signal sequence from a Synechocystis sp. PCC6803 cell and be introduced into a yeast host cell including Saccharomyces cerevisiae or Pichia pastoris, filamentous fungi host cells including Aspergillus, Trichoderma and Neurospora, mammalian host cells including murine and human, or insect host cells, and the like, to target the expressed gene product to the appropriate region of the host cell. In yet another embodiment, the synthetic operons of the invention described herein will incorporate a codon and expression optimized signal sequence from a eukaryotic cell including but not limited to a yeast cell and be introduced into a second yeast host cell including Saccharomyces cerevisiae or Pichia pastoris, bacteria including, but not limited to, Synechococcus and E. coli, filamentous fungi host cells including Aspergillus, Trichoderma and Neurospora, mammalian host cells including murine and human, or insect host cells, and the like, to target the expressed gene product to the appropriate region of the host cell.

Although the invention has been described with reference to specific embodiments and aspects presented herein, it will be understood that variations and modifications of thermophilic genes engineered into a host cell for a functional proteorhodopsin photosystem are encompassed within the spirit and scope of the invention.

### Proteorhodopsin Selection

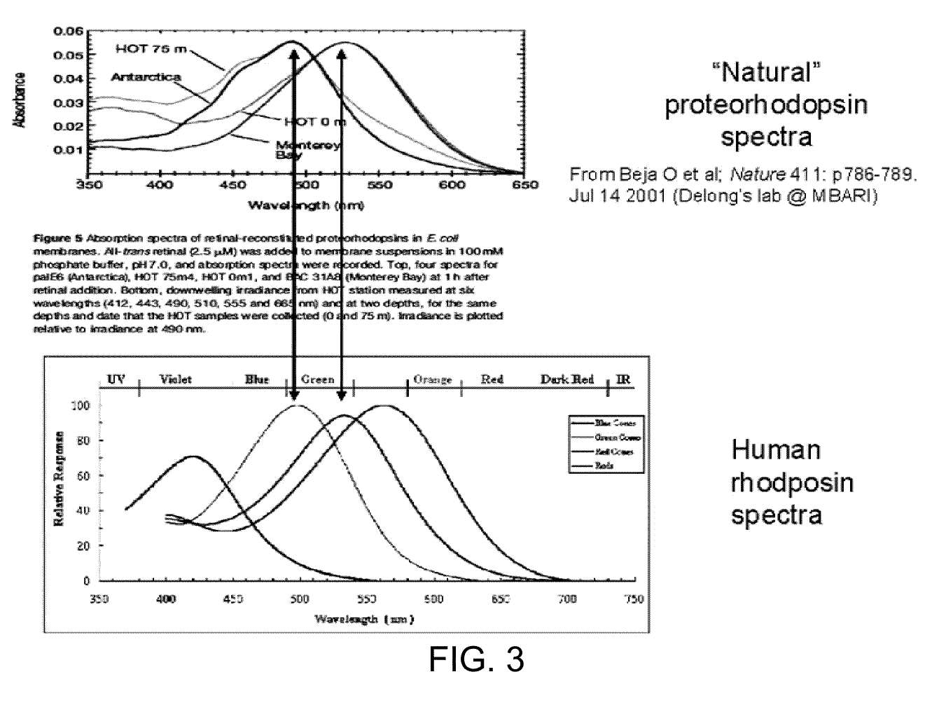

The protein pigments of the rhodopsin family appears to be spectrally tuned to different habitats-absorbing light at different wavelengths in accordance with light available in the environment (Beja et al., (2001) Nature 444:786-789) (FIG. 3). Under certain conditions proteorhodopsins may be adapted to different light intensities in their environment. A recent study suggests that proteorhodopsins were adapted to different light intensities in the marine environment via Darwinian evolution that involved substitutions of major effect and substitutions for fine-tuning of aborption maxima (Bielawski J. P., et al. (2004) Proc. Natl. Acad. Sci. USA 101: 14824-14829). It is contemplated, therefore, that the proteorhodopsins of the present invention can be selected, modified or engineered to absorb different wavelengths of light.

### Proteorhodopsin-Based Therapeutics

Photostimulation via introduction of naturally occurring light-sensitive channels and receptors, e.g., rhodopsin, has been demonstrated (Li X., (2005) Proc. Natl. Acad. Sci. USA 102:17816-17821). Accordingly, therapeutic applications based on light treatment using proteorhodopsins are also contemplated in this invention.

The examples provided herein illustrate the invention in more detail. These examples are provided to enable those skilled artisans to help understand and practice various aspects of the invention and therefore should not be construed as limiting. Various modifications and extensions of the invention in addition to those described herein will become apparent to those skilled artisans and therefore such modifications and extensions fall within the scope of invention.

### EXAMPLES

### Example 1

### E. coli Propagation

Wild-type bacteria are propagated in rich Luria-Bertani (LB) broth (10 g tryptone, 5 g yeast extract, 10 g NaCl per liter, pH 7.5-8.0) [Bertani G. J Bacteriol (1951). “Studies on lysogenesis. I. The mode of phage liberation by lysogenic Escherichia coli”. 62:293-300]. When functional CO₂-fixing pathways are engineered into E. coli, the requirements for rich media are eliminated. E. coli are propagated in minimal media, primarily minimal M9 broth (42 mM Na₂HPO₄, 24 mM KH₂PO₄, 9 mM NaCl, 19 mM NH₄Cl), 1 mM MgSO₄, 0.1 mM CaCl₂, 2.0% glucose, 0.5 μg/ml thiamine). With progressive engineering, propagation is performed with glucose levels significantly and progressively below 2% (for example, 0.1%, 0.01%, or most preferably 0% v/v). Bacteria are grown in liquid media using the above recipes, or on semi-solid plates containing agarose. Growth is analyzed quantitatively via measurement of optical density at various wavelengths. Optical density measured at a wavelength of 600 nm (OD₆₀₀) is used as a baseline measurement of growth, though additional wavelengths, including 360 nm, 420 nm, 540 nm, and 720 nm are used as corroborating values when chromophores are inserted and engineered.

E. coli is typically propagated at temperatures between 15-55° C., most typically 25-37° C. Samples of E. coli are archived indefinitely via inclusion of glycerol (typically 2-20% v/v) and stored at −80° C.

### Example 2

### Engineering Saccharomyces cerevisiae

In addition to the engineering of E. coli, the nonpathogenic and genetically tractable baker's yeast, Saccharomyces cerevisiae, is engineered. Methods for growth and manipulation are well known to those skilled in the art [J. R. Broach, E. W. Jones, and J. R. Pringle (eds.), “The Molecular and Cellular Biology of the Yeast Saccharomyces,” Vol. 1. Genome Dynamics, Protein Synthesis, and Energetics. Cold Spring Harbor Laboratory Press, Cold Spring Harbor, N.Y., 1991; E. W. Jones, J. R. Pringle, and J. R. Broach, (eds.), “The Molecular and Cellular Biology of the Yeast Saccharomyces,” Vol. 2. Gene Expression. Cold Spring Harbor Laboratory Press, Cold Spring Harbor, N.Y., 1992; J. R. Pringle, J. R. Broach, and E. W. Jones, (eds.), “The Molecular and Cellular Biology of the Yeast Saccharomyces,” Vol. 3. Cell cycle and Cell Biology. Cold Spring Harbor Laboratory Press, Cold Spring Harbor, N.Y., 1997].

S. cerevisiae is typically propagated at 20-30° C. on rich/complete media, such as YPD containing 1% Bacto-yeast extract, 2% Bacto-peptone, 2% Dextrose, 2% Bacto-agar. Alternately, defined media such as Synthetic Dextrose media (SD) comprising 20% Dextrose, 1.7% Difco Yeast nitrogenous base (lacking amino acids), 5% ammonium sulfate, plus specific essential amino acid and nutrient supplements [“drop in”] or Synthetic Complete (SC) media, containing all required amino acids or omitting one or more [“drop out” media], which proves useful during plasmid-based selections of auxotrophic mutants, can be used.

In certain instances, the same genetic sequence designed for heterologous expression in E. coli is utilized in yeast. In preferred embodiments, the DNA sequence is modified to preferred codon bias to match S. cerevisiae. Of course, irrespective of the codon bias of the open reading frames, specific non-coding elements are employed for successful propagation and expression in S. cerevisiae. Exemplary promoters include constitutive promoters GPD, KEX2, TEF1, and TDH, and inducible promoters GAL1 [Nacken V, Achstetter T, Degryse E. “Probing the limits of expression levels by varying promoter strength and plasmid copy number in Saccharomyces cerevisiae.” Gene (1996). 175(1-2):253-60]. Copy number can be modified via use of single-copy centromeric vectors or medium-to-high copy 2 micron vectors [Nacken V et al]. When biosynthetic modules are too large for propagation in plasmids, yeast artificial chromosomes (YACs) are employed. Alternately, portions of the biosynthetic pathway are serially integrated into the yeast chromosome.

Plasmids are transformed into S. cerevisiae via the lithium acetate method using the S.c. EasyComp transformation kit (Invitrogen, Carlsbad, Calif.). Alternately, S. cerevisiae are transformed via electroporation or spheroplasting, techniques known to those skilled in the art.

### Example 3

### Engineering Acetobacter

Acetobacter aceti, strain 10-8S2 from (Okumura H, Uozumi T, and Beppu T. “Construction of plasmid vector and genetic transformation system for Acetobacter aceti.” Agril. Biol. Chem. (1985). 49:1011-1017) is also engineered, using techniques known to those skilled in the art (Okumura H, Uozumi T, and Beppu T. “Construction of plasmid vector and genetic transformation system for Acetobacter aceti.” Agril. Biol. Chem. (1985). 49:1011-1017; Nakano, S, Fukaya, M, Horinouchi S. “Putative ABC Transporter Responsible for Acetic Acid Resistance in Acetobacter aceti.” Appl. And Environ. Microbiol (2006). 72(1):497-505). Acetobacter is propagated at 30° C. in YPG medium consisting of 5 g/L yeast extract, 2 g/L polypeptone, and 30 g/L glucose per liter, pH 6.5. Other rich and minimal Acetobacter media can be used including, for example, the minimal media described in U.S. Pat. No. 6,429,002 entitled “Reticulated cellulose-producing Acetobacter strains”.

### Example 4

### Fermentation Methods

In the case of an E. coli-based batch-fed fermentation system, microorganisms are also engineered to express umuC and umuD from E coli in pBAD24 under the prpBCDE promoter system through de vovo synthesis of this gene with the appropriate end-product production genes. For small scale fermentation, E. coli BL21(DE3) cells harboring pBAD24 (with ampicillin resistance and the end-product synthesis pathway) as well as pUMVC1 (with kanamycin resistance and the acetyl Co-A/malonyl CoA overexpression system) are incubated overnight at 37° C., shaken at over 200 RPM in 2 L flasks in 500 ml M9 medium in the presence of light, carbon dioxide, and supplemented with 75 μg/ml ampicillin and 50 μg/ml kanamycin until cultures reached an OD₆₀₀ of >0.8. Upon achieving an OD₆₀₀ of >0.8, cells are supplemented with 25 mM sodium propionate (pH 8.0) to activate the engineered-in gene systems for production as well as to stop cellular proliferation (through activation of umuC and umuD proteins). Induction is preferably performed for 6 hours at 30° C. After incubation, media is examined for product using GC-MS (as described in the section “Detection and Analysis of Gene and Cell Products”).

In a preferred embodiment, a fermentation is performed wherein the engineered cell takes light and carbon dioxide as its input and produces a desirable product. The carbon dioxide can be ambient sources, as well as concentrated sources, including stack gas, offgas from coal refineries, natural gas facilities, cement factories, or breweries. Carbon dioxide is added to the reaction chamber at a rate sufficient to maintain the reaction rate as desired. This may be neutral or positive pressure relative to the reaction chamber. In certain instances, the gas may require cleaning or scrubbing prior to addition into the reaction chamber

For large scale product fermentation, the engineered microorganisms are grown in 10 L, 100 L, 1000 L or larger batches, fermented and induced to express desired products based on the specific genes encoded in plasmids as appropriate. E. coli BL21(DE3) cells harboring pBAD24 (with ampicillin resistance and the end-product synthesis pathway) as well as pUMVC1 (with kanamycin resistance and the acetyl Co-A/malonyl CoA overexpression system) are incubated from a 500 ml seed culture for 10 L fermentations (5 L for 100 L fermentations) in M9 media in the presence of carbon dioxide and light at 37° C. shaken at >200 RPM until cultures reached an OD₆₀₀ of >0.8 (typically 16 hours) incubated with 50 μg/ml kanamycin and 75 μg/ml ampicillin. Media is continuously supplemented to maintain a 25 mM sodium propionate (pH 8.0) to activate the engineered-in gene systems for production as well as to stop cellular proliferation (through activation of umuC and umuD proteins). After the first hour of induction, aliquots of no more than 10% of the total cell volume are removed each hour and allowed to sit unagitated so as to allow the aqueous product to rise to the surface and undergo a spontaneous phase separation (if not possible, separation from media or cells is achieved as previously described). The hydrocarbon component is then collected and the aqueous phase returned to the reaction chamber. The reaction chamber is operated continuously. When the OD₆₀₀ drops below 0.6, the cells are replaced with a new batch grown from a seed culture.

### Example 5

### Engineering Light Capture

Light-induced proton motive force and subsequent ATP generation is assayed using several methods. First, light-dependent increases in survival is monitored in cells treated with the respiratory poison azide, as described in Walter et al, “Light-powering Escherichia coli with proteorhodopsin” PNAS (2007). 104(7):2408-2412. Second, a luciferase-based assay measuring cellular ATP levels is used to screen for cells with elevated ATP content specifically in response to light (a control is established using the same culture grown in dark); this assay is described in Martinez A et al; “Proteorhodopsin photosystem gene expression enables photophosphorylation in a heterologous host.” PNAS (2006). 104(13):5590-5595. For a full conversion, the light capture approach is combined with the CO₂ fixation approach through growth in minimal media only in presence of light.

A variety of microorganisms are known to encode light-activated proton translocation systems. In the present invention, one or more forms of light-activated proton pumps are functionally expressed in E. coli or other host cells to generate a proton gradient that is converted into ATP via an endogenous or exogenous ATPase.

Table 1 lists candidate genes for overexpression in the light capture/harvesting module together with information on associated pathways, Enzyme Commission (EC) Numbers, exemplary gene names, source organism, GenBank accession numbers, and homologs from alternate sources.

The proteorhodopsin (PR) gene is preferentially expressed in organisms. An exemplary PR sequence is locus ABL60988 described in Martinerz A, Bradley A S, Walbauer J R, Summons R E, DeLong E F. PNAS (2007). “Proteorhodopsin photosystem gene expression enables photophosphorylation in a heterologous host.” 104(13):5590-5595 with an amino acid sequence as set forth in SEQ ID NO: 1.

In addition, or as an alternative, a bacteriorhodopsin gene is expressed [Oesterhelt D, Stoeckenius W. Nature (1971) “Rhodopsin-like protein from the purple membrane of Halobacterium halobium.” 233:149-152]. An exemplary bacteriorhodopsin sequence is the NP_{—}280292 locus described in Ng W V et al. PNAS (2000). “Genome sequence of Halobacterium species NRC-1.” 97(22):12176-22181, with an amino acid sequence as set forth in SEQ ID NO: 2. Bacteriorhodopsin has previously been functionally expressed in yeast mitochondria [Hoffmann A, Hildebrandt V, Heberle J, Buldt G. “Photoactive mitochondria: In vivo transfer of a light-driven proton pump into the inner mitochondrial membrane of Schizosaccharomyces pombe.” Proc. Natl. Acad. Sci. (1994). 91: 9637-71].

Similarly, deltarhodopsin is expressed in addition to or as an alternative [Ihara K et al. J Mol Biol (1999). “Evolution of the archael rhodopsins: evolution rate changes by gene duplication and functional differentiation.” 285:163-174; Kamo N, Hashiba T, Kikukawa T, Araiso T, Ihara K, Nara T. Biochem Biophys Res Commun (2006). “A light-driven proton pump from Haloterrigena turkmenica: functional expression in Escherichia coli membrane and coupling with a H⁺ co-transporter.” 342(2): 285-90). An exemplary deltarhodopsin sequence is the AB009620 locus of Haloterrigena sp. Arg-4 described in Ihara K et al. J Mol Biol (1999). “Evolution of the archael rhodopsins: evolution rate changes by gene duplication and functional differentiation.” 285:163-174, with an amino acid sequence as set forth in SEQ ID NO: 3.

Similarly, the Leptosphaeria maculans opsin protein is expressed as an addition to or as an alternative to other proton pumps. An exemplary eukaryotic light-activated proton pump is opsin, accession AAG01180 from Leptosphaeria maculans, described in Waschuk S A, Benzerra A G, Shi L, and Brown L S. PNAS (2005). “Leptosphaeria rhodopsin: Bacteriorhodopsin-like proton pump from a eukaryote.” 102(19):6879-83], with an amino acid sequence as set forth in SEQ ID NO: 103.

Finally a xanthorhodopsin proton pump with a carotenoid antenna is expressed in addition to or as an alternative to other proton pumps (Balashov S P, Imasheva E S, Boichenko V A, Anton J, Wang J M, Lanyi J K. Science (2005) “Xanthorhodopsin: A proton pump with a light harvesting cartenoid antenna.” 309(5743): 2061-2064). An exemplary xanthorhodopsin sequence is locus ABC44767 from Salinibacter ruber DSM 13855 described in Mongodin E F et al. PNAS (2005). “The genome of Salinibacter ruber: Convergence and gene exchange among hyperhalophilic bacteria and archaea.” 102(50):18147-18152, with an amino acid sequence as set forth in SEQ ID NO: 4.

The pumps are used alone or in combination, optimized to the specific cell. The pumps can be directed to be incorporated into one or more than one membrane location, for example the cytoplasmic, outer membrane, or mitochondrial membrane. Xanthorhodopsin and proteorhodopsin co-expression represents an optimal combination.

In addition to the expression of one or more proton pumps described above, a retinal biosynthesis pathway can be expressed. When PR and the retinal biosynthetic operon are functionally expressed in E. coli, the pump is able to restore proton motive force to azide-treated E. coli populations [Walter J M, Greenfield D, Bustamante C, Liphardt J. PNAS (2007). “Light-powering Escherichia coli with proteorhodopsin.” 104(7):2408-2412]. A six gene retinal biosynthesis operon, Accession number EF100190 is known (Martinerz A, Bradley A S, Walbauer J R, Summons R E, DeLong E F. PNAS (2007). “Proteorhodopsin photosystem gene expression enables photophosphorylation in a heterologous host.” 104(13):5590-5595) which encodes amino acid sequences set forth in SEQ ID NO: 5 (Isopentenyl-diphosphate delta-isomerase (Idi), locus ABL60982), SEQ ID NO: 6 (15,15′-beta-carotene dioxygenase (Blh), locus ABL60983), SEQ ID NO: 7 (Lycopene cyclase (CrtY), locus ABL60984), SEQ ID NO: 8 (Phytoene synthase (CrtB), EC 2.5.1.32, locus ABL60985), SEQ ID NO: 9 (Phytoene dehydrogenase (CrtI), locus ABL60986), and SEQ ID NO: 10 (Geranylgeranyl pyrophosphate synthetase (CrtE), locus ABL60987).

The above 6 enzymes enable biosynthesis of retinal, which is the essential chromophore common to all rhodopsin-related proton pumps. In certain embodiments, additional spectral absorption is provided by carotenoids, as exemplified by the xanthorhodopsin pump and the C-40 salinixanthin antenna. In these embodiments, a beta-carotene ketolase (CrtO) is expressed, such as the crtO gene of the SRU_{—}1502 locus in Salinibacter ruber, described in Mongodin E F et al (2005), with an amino acid sequence as set forth in SEQ ID NO: 11. Other crtO genes include those from Rhodococcus erythropolis (AY705709), with an amino acid sequence as set forth in SEQ ID NO: 104, and Deinococcus radiodurans R1 (NP_{—}293819), with an amino acid sequence as set forth in SEQ ID NO: 122.

With a functional PR module expressed, the natural respiratory pathways are redundant. Thus, a plurality of endogenous genes can be disrupted including NADH dehydrogenase I (14 gene nuo operon, nuoA-N), NADH dehydrogenase II (ndh), and the cytochrome quinol oxidases (cyo and cyd).

Nuo proteins typically transfer electrons from NADH to ubiquinone in the electron transfer chain and produce a proton motive force. Mutants are typically deficient in energy generation and exhibit a significantly increased ratio of reduced (NADH) to oxidized (NAD⁺) pyridine nucleotide pools [Gennis R B and Stewart V. Respiration, p 217-261. In Neidhardt F C et al. Escherichia coli and Salmonella: cellular and molecular biology, vol 1. ASM Press, Washington D.C.; Claas K, Weber S, Downs D M. J Bacteriol (2000). “Lesions in the nuo operon, encoding NADH dehydrogenase complex I, prevent PurF-independent thiamine synthesis and reduce flux through the oxidative pentose phosphate pathway in Salmonella enterica serovar typhimurum.” 182(1):228-23]. The increased NADH concentration is important in the context of the present invention, because it provides the reducing power necessary for carbon fixation.

Proteorhodopsin Plasmid

The plasmid PtrcHis2origPR-N (pJB304), a pBR322-derivative with a beta-lactamase (bla) cassette bearing the SAR86 proteorhodopsin (PR) gene (Genbank: AF279106, (Beja, O., & others. (2000). Bacterial Rhodopsin: Evidence for a New Type of Phototrophy in the Sea. Science, 1902-1906) under the control of the Ptrc promoter, was provided by Jessica Walters and Jan Liphardt (University of California, Berkeley).

Phosphoribulokinase, RUBISCO Genes and Plasmids

The phosphoribulokinase gene prkA from Synechococcus sp. PCC7942 (Genbank: AB035257) was obtained from DNA 2.0 following codon optimization, checking for secondary structure effects, and removal of any unwanted restriction sites (SEQ ID NO 271). The gene was obtained with NcoI and BamHI restriction upstream of the gene and a HindIII restriction site downstream. The rbcL and rbcS genes from Synechococcus sp. PCC7942 (Genbank: NC_{—}006576) were also obtained from DNA 2.0 following codon optimization and correcting for secondary structure effects (see SEQ ID NOs 272-277). They were constructed in an operon with a NdeI site upstream of rbcL, SacI and SbfI restriction sites placed in between rbcL and rbcS, and a XhoI site placed downstream of rbcS. Another rbcL variant (rbcL1_{—}15) contained Met259Thr, a mutation which was shown to have five-fold greater specific activity in E. coli (Parikh, M. R., N., G. D., Woods, K. K., & Matsumura, I. (2006). Directed Evolution of RuBisCO hypermorphs through genetic selection in engineered E. coli. Protein Engineering, Design & Selection, 113-119) was made as well in the identical operon as rbcLS. prkA was digested with NcoI and BamHI and ligated into the MCS1 of a similarly-digested pCDFDuet-1 (Novagen, now EMD Chemicals) to yield pJB265. pCDFDuet-1 has a compatible origin of replication (CDF ori) and resistance cassette (aadA) for co-expression with PtrcHis2origPR-N. The rbcL1_{—}15S and rbcLS genes were cloned into MCS2 of pJB265 using the NdeI-XHoI sites to generate pJB267 and pJB268, respectively.

Strains

The E. coli strain BL21 DE(3) (Invitrogen) was used for expression studies, and the following strains were prepared by transformation of the respective plasmids into this host (Table 2):

TABLE 2

| BL21 DE(3) strains | Plasmids | Genes |
|---|---|---|
| JCC308 | pCDFDuet-1 | — |
| JCC309 | pJB285 | prkA |
| JCC311 | pJB267 | prkA, rbcL1_15S |
| JCC312 | pJB268 | prkA, rbcLS |
| JCC349 | pJB304, pCDFDuet-1 | PR, — |
| JCC351 | pJB304, pJB267 | PR, prkA, rbcL1_15S |
| JCC352 | pJB304, pJB268 | PR, prkA, rbcLS |

Expression of Proteorhodopsin

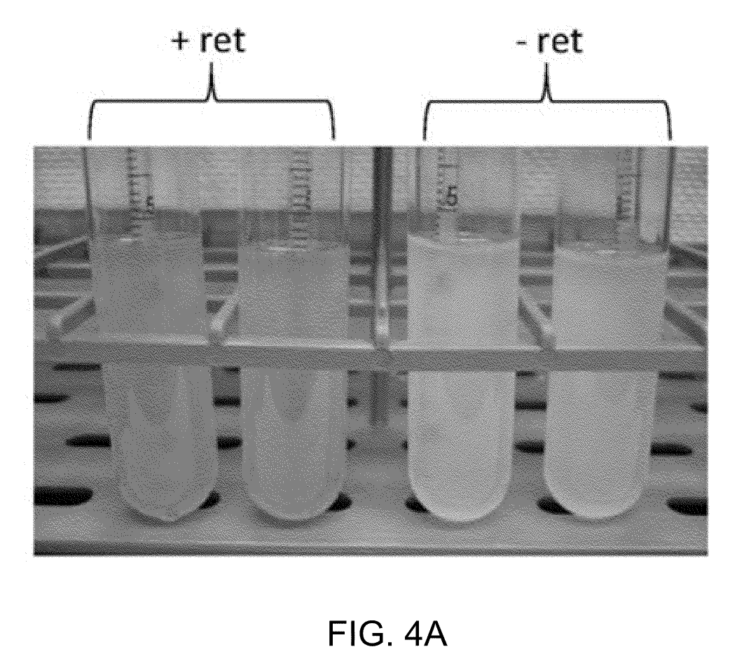

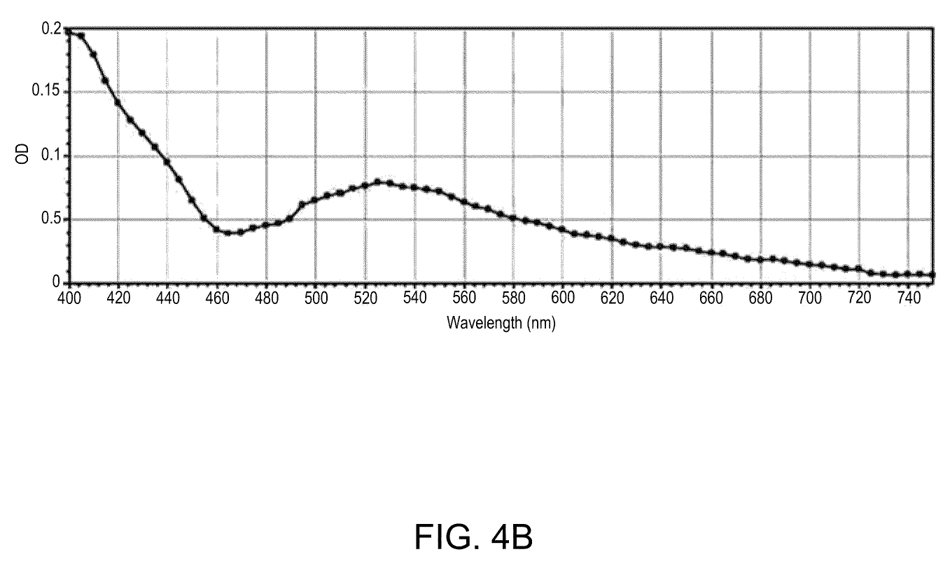

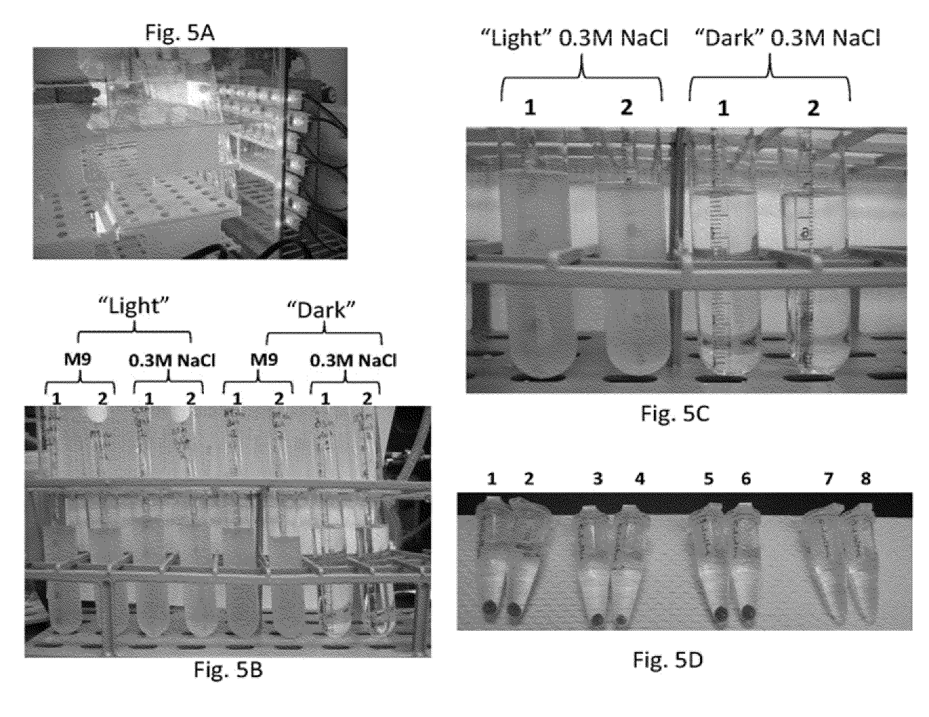

The strain JCC349 (pJB304, pCDFDuet-1) was induced at OD₆₀₀=0.1-0.2 with 0.1 mM IPTG in LB with 100 μg/ml carbenicillin and 50 μg/ml spectinomycin. Two cultures were induced, one with 20 μM trans-retinal added (from 20 mM trans-retinal in ethanol) and the other supplemented with an equal volume of ethanol, for a total of six hours. The cells were pelleted using a Sorvall RC6 Plus superspeed centrifuge (Thermo Electron Corp) and a F13S-14X50CY rotor (5000 rpm for 10 min). The cells induced with retinal present were red as expected with the proteorhodopsin holoprotein being present (Beja & others, 2000) and those cells induced without retinal present were white, indicating the presence of the apoprotein (Beja & others, 2000). Cells were resuspended in M9 minimal media/0.2% L-arabinose with 100 μg/ml carbenicillin and 50 μg/ml spectinomycin, and pelleted using an Eppendorf Centrifuge 5424 microcentrifuge (1 min, 15000 rpm). The M9 minimal media used in these experiments contained additional salt (5 g/L NaCl instead of 0.25 g) and iron (3 mg FeSO₄ heptahydrate/L). The cells were resuspended in M9/0.2% L-arabinose with 100 μg/ml carbenicillin and 50 μg/ml spectinomycin, and added to duplicate test tubes (Pyrex No. 9820, Fisher Scientific) equipped with a hollow glass rod and foam plug containing 20 mls of M9/0.2% L-arabinose, 100 μg/ml carbenicillin, 50 μg/ml spectinomycin and 0.1 mM IPTG at an OD₆₀₀=0.016. These cultures were incubated at 37° C. for 44 h. The cultures inoculated from retinal-containing culture were supplemented with 20 μM trans-retinal at t=0 and approximately every 12 h afterwards until the end of the experiment (t=44 h, OD₆₀₀=1.2-1.5, in stationary phase), while only the vector (ethanol) was added to the cultures inoculated from the other (retinal minus) induced culture at the same time. During this experiment, cultures were grown in aquaria at 37° C. with 1% CO₂/air bubbling through the glass rod at a rate of 1-2 bubbles/sec. After 44 h, the cultures containing trans-retinal were red (FIG. 4A) indicating that proteorhodopsin was still being expressed. A visible light absorbance scan was taken on a Spectramax M2 (Molecular Devices) from 400 to 750 nm on a retinal-supplemented culture using a retinal minus culture as the reference (blank), taking a reading every 5 nm (FIG. 4B). A broad peak with an absorbance maximum of approximately 520 nm was present, as expected for the proteorhodopsin holoprotein (Beja & others, 2000).

### Light Conferred Growth at an Elevated Salt Concentration

Seven green LED strips emitting at 518 nm (LB2-G12, superbrightleds.com) were connected in series and wired to a 12 VDC power supply (CPS-24, superbrightleds.com). The emitted light was measured using a LI-250A light meter (LI-COR) which can sense PAR (photosynthetically active radiation, 400-700 nm) was 20-80 μE/m²s as the meter was moved across the board at about 1 inch distance from the LED board. The LED board was attached to the side of an aquarium inside which test tube racks were placed to hold the test tubes containing cultures close to the lights (see FIG. 5A). The PAR received by a culture inside a glass tube illuminated by the LED board, measured by an immersible probe (Quantum Scalar Laboratory irradiance sensor, BioSpherical Instruments Inc.), varied from 20-30 μE/m²s as the sensor was moved from bottom to top of the glass tube. A culture of JCC349 (PR, pCDFDuet-1) was induced with 0.1 mM IPTG in the presence of 20 μM trans-retinal for 7 h in the manner described above, and innoculated at a starting OD₆₀₀=0.01 into two set of aquarium culture tubes containing 20 mls of M9 minimal media/0.2% L-arabinose, 0.1 mM IPTG and 20 μM trans-retinal. Both sets contained duplicate cultures with no additional salt, 0.3M sodium chloride, 0.5 M sodium chloride and 1M sodium chloride. One set was illuminated with the green LED bank described above, and the other set was kept in the dark in the same aquarium. The “dark” cultures did receive some ambient light, determined to be 0.5 μE/m²s when measured with the immersible sensor. All cultures were incubated at 37° C. and bubbled at a rate of 1-3 bubbles/sec with 1% CO₂/air. Trans-retinal was added to a concentration of 20 μM to each culture twice a day (about every 12 h). After 61 hours, the “light” cultures in M9 media and the media supplemented with 0.3 M sodium chloride grew, where the “dark” cultures only showed growth in the unsupplemented M9 media (FIGS. 5B, 5C). Optical densities at 600 nm were taken on a Spectramax M2 (Molecular Devices) for the cultures in M9 media and supplemented with 0.3 M NaCl (Table 3). 5 mls of each culture was pelleted, the media discarded, the cells washed in 1 ml milli-Q water (FIG. 5D), and the supernatant discarded. The pellets were then frozen, dried overnight under vacuum, and dry weights were recorded (Table 3).

TABLE 3
Table 3. OD₆₀₀ and dry weights of JCC349 grown in M9
minimal media and M9 supplemented with 0.3 M NaCl
under green light or in the dark.

| “Light” | | Dry weight | “Dark” | | Dry weight |
|---|---|---|---|---|---|
| culture | OD₆₀₀ | (mg/5 ml) | culture | OD₆₀₀ | (mg/5 ml) |
| M9 #1 | 1.3 | 2.7 | M9 #1 | 1.4 | 3.2 |
| M9 #2 | 1.4 | 2.9 | M9 #2 | 1.5 | 3.4 |
| 0.3M | 0.95 | 1.8 | 0.3M NaCl #1 | 0.08 | 0 |
| NaCl #1 | | | | | |
| 0.3M | 0.63 | 1.0 | 0.3M NaCl #2 | 0.08 | 0 |
| NaCl #2 | | | | | |

Expression of prkA and RUBISCO Genes in E. coli

Expression of phosphoribulokinase A, rbcL and rbcS has previously been demonstrated in E. coli. Expression of prkA is toxic, believed to be caused by a buildup of D-ribulose-1,5-bisphosphate which is not metabolized by E. coli (Parikh, N., Woods, & Matsumura, 2006). Expression of rbcLS with prkA allowed growth through production of 3-phosphoglycerate from D-ribulose-1,5-bisphosphate, but required CO₂ supplementation (Parikh, N., Woods, & Matsumura, 2006).

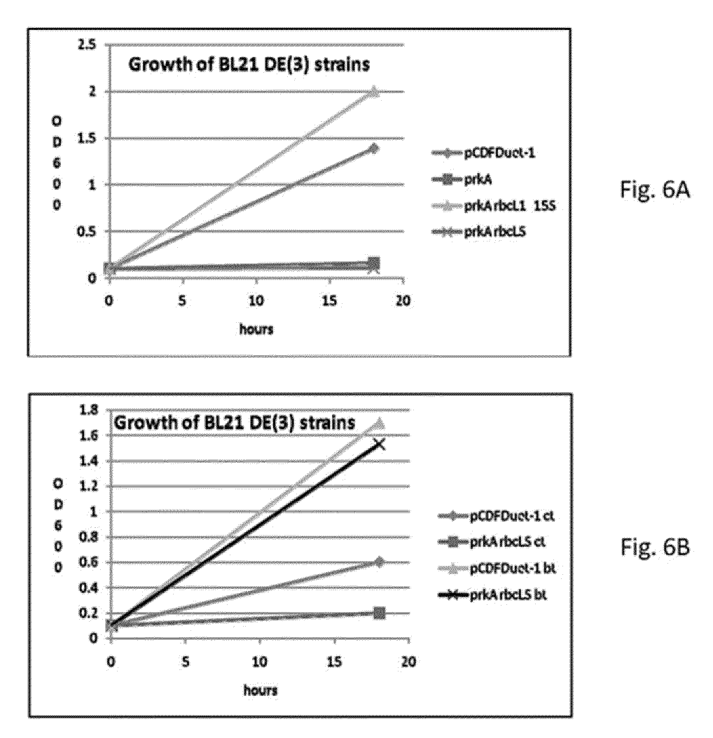

Strains JCC308 (pCDFDuet-1), JCC309 (prkA), JCC311 (prkA rbcL1_{—}15S), and JCC312 (prkA rbcLS) were induced in LB/spectinomycin (50 μg/ml) with 0.1 mM IPTG at an OD₆₀₀=0.2-0.4 for 3 hours. Cells were washed with M9/0.2% L-arabinose, and resuspended in 4 mls of M9/0.2% L-arabinose, spectinomycin (50 μg/ml), 0.1 mM IPTG. Cells were incubated for about 18 h in a shaking incubator at 37° C. and OD₆₀₀ values were recorded (FIG. 6A). The JCC309 cells which expressed prkA did not grow on L-arabinose, as expected (Parikh, N., Woods, & Matsumura, 2006). JCC312 also failed to grow, possibly due to insufficient levels of carbon dioxide being present for RbcLS to convert enough D-ribulose-1,5-bisphosphate to 3-phosphoglycerate for growth to occur. JCC311 did grow, suggesting that the optimized RbcLS enzyme (rbcL1_{—}15S) could metabolize enough D-ribulose-1,5-bisphosphate under these conditions to allow growth.

In order to test whether carbon dioxide supplementation would allow growth, JCC308 and JCC312 were induced in LB/spectinomycin (50 μg/ml) with 0.1 mM IPTG at an OD₆₀₀=0.2-0.4 for 3 hours. Cells were washed with M9/0.2% L-arabinose containing spectinomycin (50 μg/ml), and resuspended in 14 mls of M9/0.2% L-arabinose, spectinomycin (50 μg/ml) and 0.1 mM IPTG to an OD₆₀₀=0.04. 4 mls were incubated for about 18 h in a shaking incubator at 37° C. and 10 mls of each culture were incubated in a bubble tube at 37° C. where 1% CO₂/air was bubbled through at 1-2 bubbles/second. OD₆₀₀ values were recorded following the experiment (FIG. 6B). Comparison of the cultures grown under the different conditions showed that after 18 h JCC308 (pCDFDuet-1) and JCC312 (prkA rbcLS) had achieved approximately the same cell density when bubbled with 1% CO₂/air, but not in the culture tubes where JCC312 was 1/3 the density of JCC308. This is consistent with the previously reported research (Parikh, N., Woods, & Matsumura, 2006) that CO₂ supplementation is important for E. coli to grow when expressing prkA and rbcLS and growing on L-arabinose and verifies function of the enzymes.

Co-Expression of Proteorhodopsin, prkA and RUBISCO Genes in E. coli

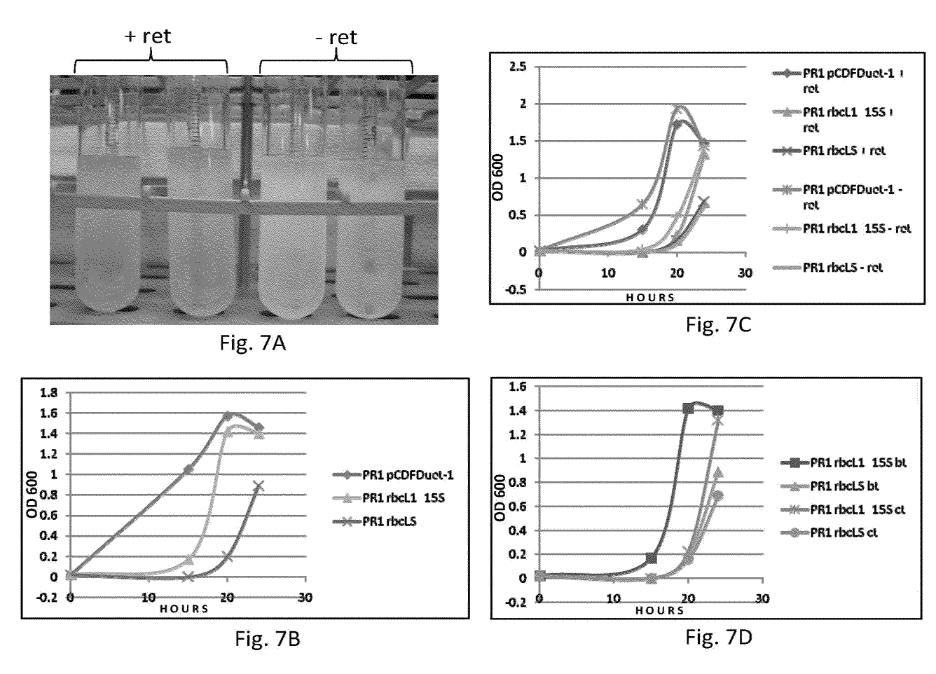

JCC351 (PR prkA rbcL1_{—}15S) and JCC352 (PR prkA rbcLS) was induced and grown as described for JCC349 in Expression of Proteorhodopsin. After 44 h incubation in M9/0.2% arabinose, both JCC351 and JCC352 were red when supplemented with trans-retinal (for picture of JCC351 duplicates incubated with and without trans-retinal, see FIG. 7A) indicating that proteorhodopsin is expressed functionally when co-expressed with prkA and RUBISCO genes.

To test expression of prkA and rbcL1_{—}15S and effect of trans-retinal on growth, cultures of JCC349 (PR pCDFDuet-1), JCC351 (PR prkA rbcL1_{—}15S) and JCC352 (PR prkA rbcLS) were induced at OD₆₀₀=0.1-0.2 with 0.1 mM IPTG in LB with 100 μg/ml carbenicillin and 50 μg/ml spectinomycin. Two cultures were induced, one with 20 μM trans-retinal added (from 20 mM trans-retinal in ethanol) and the other supplemented with an equal volume of ethanol, for a total of 6 hours. The cells were pelleted using a Sorvall RC6 Plus superspeed centrifuge (Thermo Electron Corp) and a F13S-14X50CY rotor (5000 rpm for 10 min). The cells induced with retinal present were red as expected with the proteorhodopsin holoprotein being present (Beja & others, 2000) and those cells induced without retinal present were white, indicating the presence of the apoprotein (Beja & others, 2000). Cells induced were resuspended in M9 minimal media*/0.2% arabinose with 100 μg/ml carbenicillin and 50 μg/ml spectinomycin, and pelleted using an Eppendorf Centrifuge 5424 microcentrifuge (1 min, 15000 rpm). The cells were resuspended in M9/0.2% arabinose with 100 μg/ml carbenicillin and 50 μg/ml spectinomycin, and the cultures induced with retinal were added to test tubes (Pyrex No. 9820, Fisher Scientific) equipped with a hollow glass rod and foam plug containing 10 mls of M9/0.2% arabinose, 100 μg/ml carbenicillin, 50 μg/ml spectinomycin and 0.1 mM IPTG at an OD₆₀₀=0.02. ml cultures were started in the same media and placed in a 37° C. shaking incubator for both cultures induced in the presence and absence of trans-retinal at the same OD₆₀₀. During this experiment, cultures were grown in aquaria at 37° C. with 1% CO₂/air bubbling through the glass rod at a rate of 1-2 bubbles/sec. All cultures were incubated for 24 h, taking OD₆₀₀ measurements at t=15 h, 20 h and 24 h. The cultures inoculated from retinal-containing culture were supplemented with 20 μM trans-retinal at t=0 and approximately every 12 h afterwards until the end of the experiment (t=24 h) to check for red cell color, while only the vector (ethanol) was added to the cultures innoculated from the other (retinal minus) induced culture at the same time.

Growth in the aquarium bubble tubes followed the same trend as observed previously when the prkA and RUBISCO genes were expressed without proteorhodopsin, with JCC349 growing first followed by JCC351 and JCC352 (FIG. 7B). The same trend was observed in the culture tubes (FIG. 7C). Cultures grown with trans-retinal have similar growth curves with those lacking trans-retinal (FIG. 7C), confirming the assumption that addition of trans-retinal provides no growth benefit without light. Comparison of the JCC351 and JCC352 growth curves in the bubble tubes and culture tubes (FIG. 7D) revealed that the JCC351 came out of lag phase and reached stationary phase faster than the other three culture. This indicates that JCC351 (PR prkA rbcL1_{—}15S) has improved growth with supplemented CO₂, as would be expected for RUBISCO in the conversion of 3-phosphoglycerate from D-ribulose-1,5-bisphosphate (Parikh, N., Woods, & Matsumura, 2006). Less of an effect was noticed with JCC352 (PR prkA rbcLS), but the strain did appear to be growing slightly faster in the bubble tube than the culture tube.

Carbon Fixation Experiment in E. coli

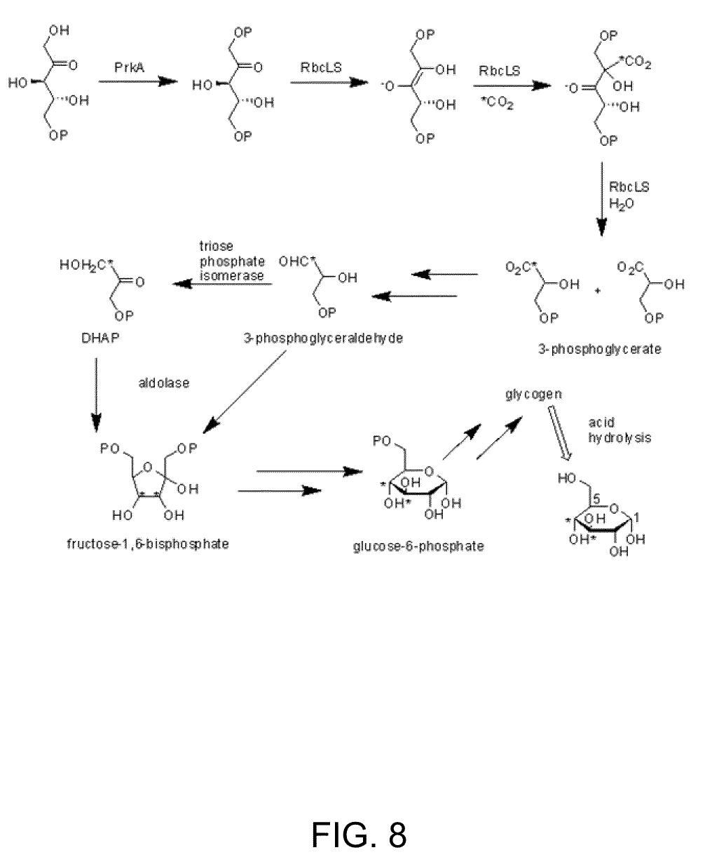

In order to test for carbon fixation by JCC350 and JCC351, the cells are incubated in M9/0.2% L-arabinose with lower concentrations of ammonium chloride added (a condition known to trigger glycogen production in E. coli when nitrogen limitation is reached (for example, see Dietzler, D. N. (1973). Rates of Glycogen Synthesis and the Cellular Levels of ATP and FDP During Exponential Growth and Nitrogen-Limited Stationary Phase of Escherichia coli W4597 (K). Arch. Biochem. Biophys., 684-693.). ¹³C-labelled sodium bicarbonate is added to media, and uptake of ¹³CO₂ into glycogen via the gluconeogenesis pathway from 3-phosphoglycerate (the product of phosphoribulokinase A (prkA) and RUBISCO from D-ribulose-5-phosphate which is generated from L-arabinose metabolism by E. coli). Glycogen is isolated from these cells using a standard procedure of cell lysis with B-PER II (Pierce) and ethanol precipitation of glycogen after treatment with a DNase. The purified glycogen would be subjected to acid hydrolysis followed by ¹³C NMR and MS analysis to measure ¹³C incorporation in the obtained glucose. Two carbon positions in glucose are anticipated to be ¹³C-labelled in this approach (FIG. 8) leading to population of differently labeled glucose molecules (not considering α- and β-isomers). Without prkA and RUBISCO, L-arabinose would likely be incorporated into glycogen via the pentose phosphate pathway and this labeling pattern would be found.

### Example 6

### Engineering Carbon Fixation

Cells engineered to contain a functional CO₂ fixation pathway are selected for via growth in minimal media lacking an organic carbon source. Exemplary modes for supplying CO₂ include bubbling directly into media, aeration in the presence of a atmosphere containing concentrated CO₂, or via inclusion of bicarbonate in media formulations. While all cells will survive in rich media (such as LB or 2xYT) or in minimal media containing glucose or other organic carbon sources, only autotrophic cells will survive in minimal media containing CO₂ as the sole carbon source. Selection for autotrophic cells can be immediate (i.e., cells are plated or inoculated directly into minimal media) or can be gradual (i.e., cells are placed in a chemostat, and minimal media containing exogenous sugar is gradually replaced with minimal media containing only CO₂). In addition to survival-based selections, cells can be grown in minimal media in the presence of radiolabeled CO₂ (i.e., C¹⁴—CO₂). Detailed incorporation studies are employed to verify and characterize metabolic assimilation using common techniques known to those skilled in the art.

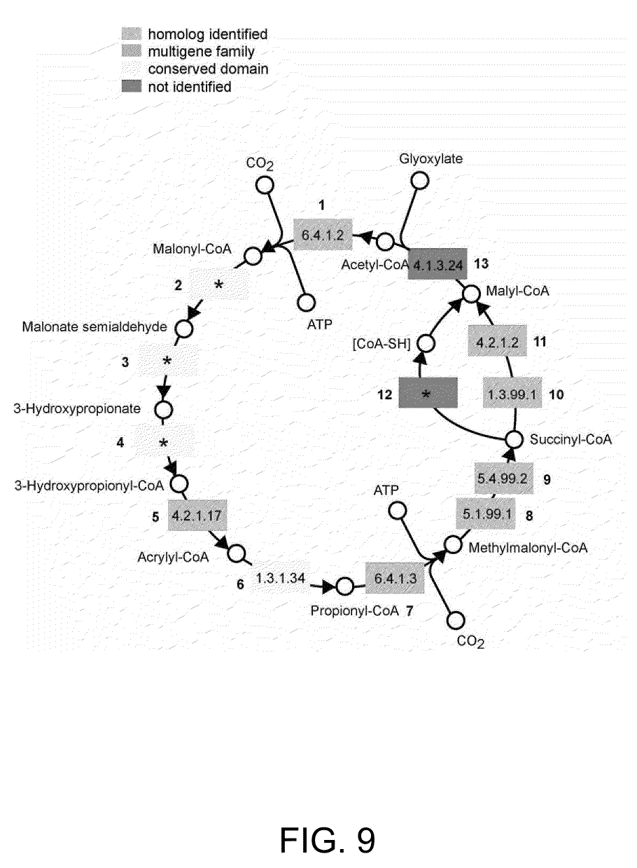

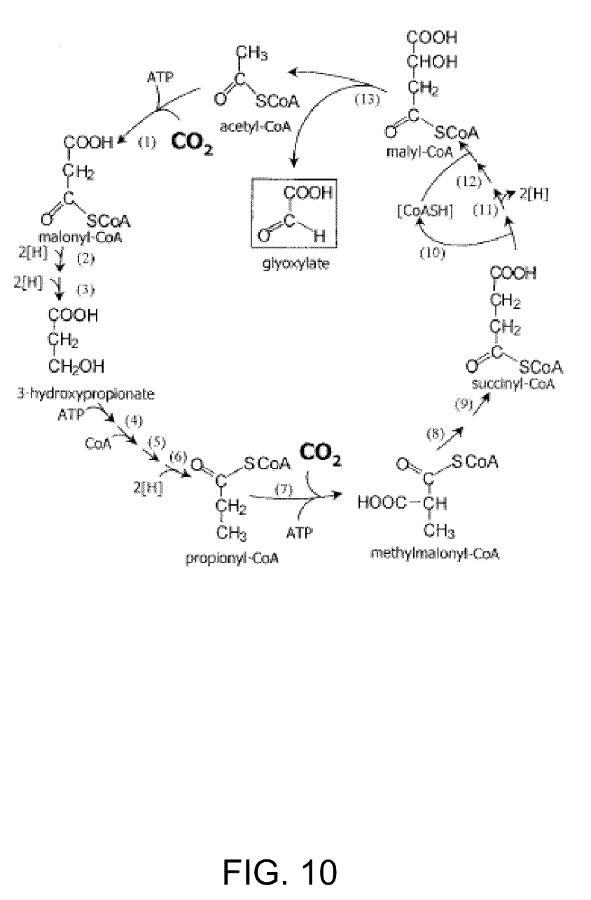

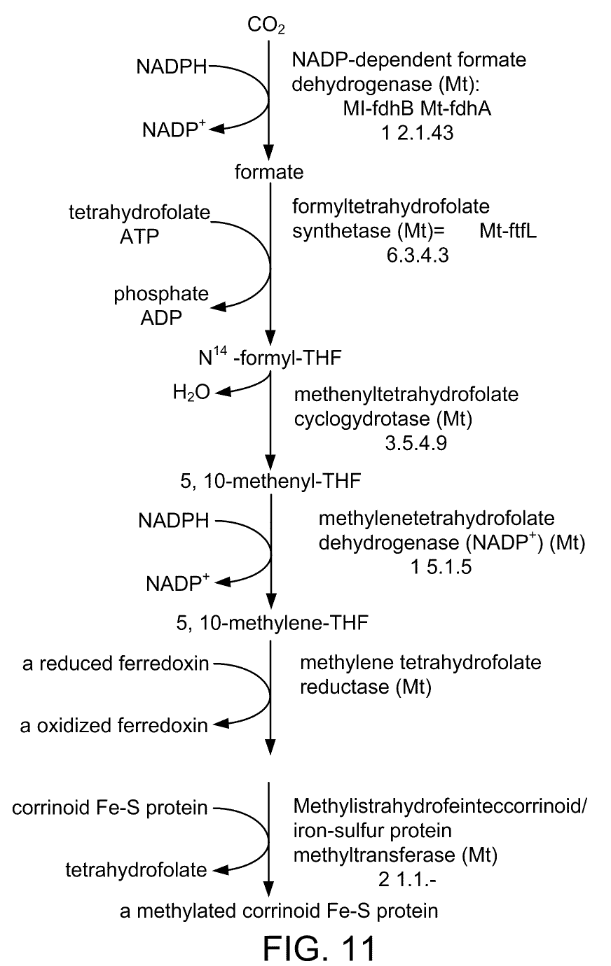

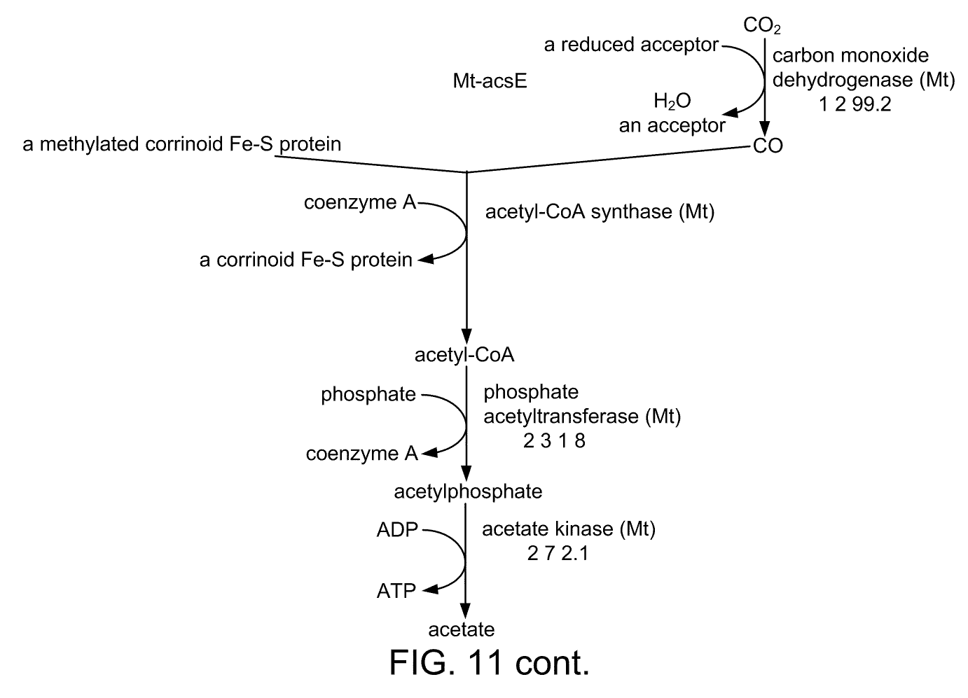

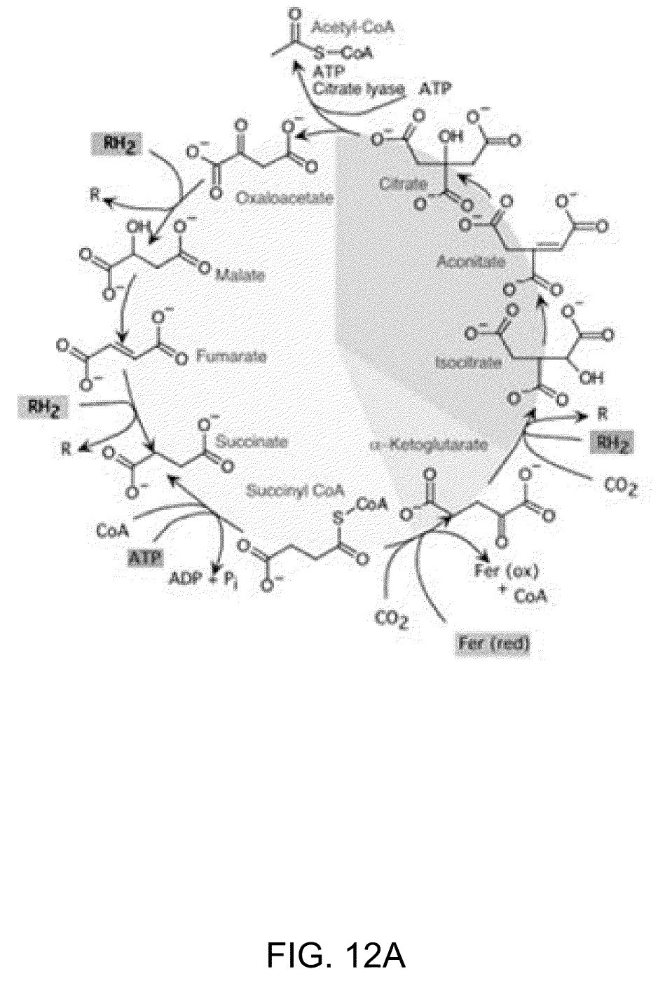

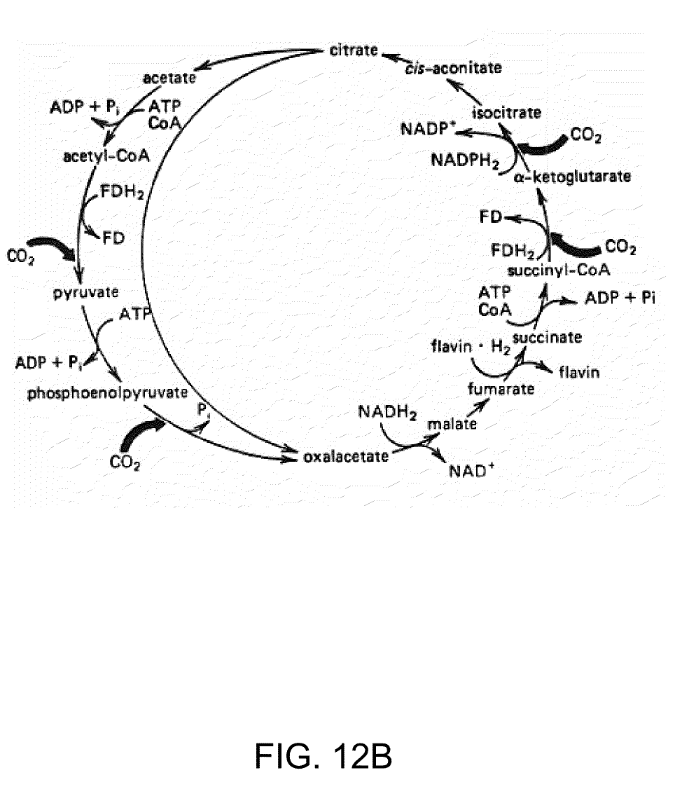

There are four known pathways that enable autotrophic carbon fixation. Cells are can be engineered to express the genes needed for the 3-hydroxyproprionate (3-HPA) cycle (FIG. 9, FIG. 10). Cells optionally can be engineered to express the genes needed for the reductive TCA cycle (FIG. 12). The genes encoding the reductive acetyl coenzyme A pathway (also known as Woods-Ljungdahl pathway) also can be engineered into cells (FIG. 11). Combinations of these (preferentially the 3-HPA cycle and the reductive TCA cycle) can also be engineered in special cases. Alternately, it is recognized that Rubisco and associated enzymes comprising the dark cycle of photosynthesis (also known as the reductive pentose phosphate cycle or the Calvin-Benson cycle) can be engineered into host organisms. However, given known problems related to efficiency and a reliance on extensively invaginated membrane structures, the reductive pentose phosphate cycle is not the preferred embodiment. Nonetheless, it is recognized that this cycle does represent an alternative to theoretically achieve the objective of enabling autotrophic carbon fixation.

Table 1 lists candidate genes for overexpression in the carbon fixation modules together with information on associated pathways, Enzyme Commission (EC) Numbers, exemplary gene names, source organism, GenBank accession numbers, and homologs from alternate sources.

I. Enzymes for a Functional 3-hydroxypropionate Cycle

The following enzyme activities are expressed in E. coli to establish a functional 3-hydroxypropionate cycle. This pathway is employed by Chloroflexus aurantiacus [Herter S, Farfsing J, Gad'On N, Rieder C, Eisenreich W, Bacher A, and Fuchs G. J Bacteriol (2001). “Autotrophic CO₂ fixation by Chloroflexus aurantiacus: study of glyoxylate formation and assimilation via the 3-hydroxypropionate cycle.” 183(14):4305-16] (FIG. 10).

Acetyl-CoA carboxylase (ACCase), (EC 6.4.1.2), generates malonyl-CoA, ADP, and Pi from Acetyl-CoA, CO₂, and ATP. E. coli encodes a heterohexameric acetyl-CoA carboxylase, though in preferred embodiments it is useful to overexpress these components to improve CO₂ fixation. In most preferred embodiments, when E. coli encodes an endogenous gene with the desired activity, it is useful to overexpress an exogenous gene, which allows for more explicit regulatory control in the fermentation and a means to potentially mitigate the effects of central metabolism regulation, which is focused around the native genes explicity. An exemplary ACCase subunit alpha is accA from E. coli, locus AAA70370 with an amino acid sequence as set forth in SEQ ID NO: 12. An exemplary ACCase subunit beta is accD from E. coli, locus AAA23807 with an amino acid sequence as set forth in SEQ ID NO: 13. An exemplary biotin-carboxyl carrier protein is accB from E. coli, locus ECOACOAC with an amino acid sequence as set forth in SEQ ID NO: 14. An exemplary biotin carboxylase is accC from E. coli, locus AAA23748 with an amino acid sequence as set forth in SEQ ID NO: 15.

Malonyl-CoA reductase (also known as 3-hydroxypropionate dehydrogenase) (EC 1.1.1.59), generates 3-hydroxyproprionate, 2 NADP⁺, and CoA from malonyl-CoA and 2 NADPH. An exemplary bifunctional enzyme with both alcohol and dehydrogenase activities is mcr from Chloroflexus aurantiacus, locus AY530019 with an amino acid sequence as set forth in SEQ ID NO: 16.

3-hydroxypriopionyl-CoA synthetase (also known as 3-hydroxypropionyl-CoA dehydratase, or acryloyl-CoA reductase) generates propionyl-CoA, AMP, PPi (inorganic pyrophosphate), H₂O, and NADP⁺ from 3-hydroxypriopionate, ATP, CoA, and NADPH. An exemplary gene is propionyl-CoA synthase (pcs) from Chloroflexus aurantiacus, locus AF445079 with an amino acid sequence as set forth in SEQ ID NO: 17.

Propionyl-CoA carboxylase (EC 6.4.1.3) generates S-methylmalonyl-CoA, ADP, and Pi (inorganic phosphate) from Propionyl-CoA, ATP, and CO₂. An exemplary two subunit enzyme is propionyl-CoA carboxylase alpha subunit (pccA) from Roseobacter denitrificans, locus RD1_{—}2032 with an amino acid sequence as set forth in SEQ ID NO: 18 and propionyl-CoA carboxylase beta subunit (pccB) from Roseobacter denitrificans, locus RD1_{—}2028 with an amino acid sequence as set forth in SEQ ID NO: 19.

Methylmalonyl-CoA epimerase (EC 5.1.99.1) generates R-methylmalonyl-CoA from S-methylmalonyl-CoA. An exemplary enzyme from Rhodobacter sphaeroides is locus CP000661 with an amino acid sequence as set forth in SEQ ID NO: 20.

Methylmalonyl-CoA mutase (EC 5.1.99.2) generates succinyl-CoA from R-methylmalonyl-CoA. E. coli encodes an enzyme with this activity (yliK), though in preferred embodiments it is useful to overexpress this enzyme to improve CO₂ fixation. The yliK protein (locus NC000913.2) has an amino acid sequence as set forth in SEQ ID NO: 21.

Succinyl-CoA:L-malate CoA transferase generates L-malyl-CoA and succinate from succinyl-CoA and malate. An exemplary two subunit enzyme is SmtA from Chloroflexus aurantiacus, locus DQ472736.1 with an amino acid sequence as set forth in SEQ ID NO: 22 and SmtB from Chloroflexus aurantiacus, locus DQ472737.1 with an amino acid sequence as set forth in SEQ ID NO: 23.

Fumarate reductase (EC 1.3.1.6) generates fumarate and NADH from succinate and NAD⁺. Locus J01611 in E. coli is a fumarate reductase (frd) operon. In preferred embodiments, it is useful to overexpress these components to improve CO₂ fixation. The frdA fumarate reductase flavoprotein subunit has an amino acid sequence as set forth in SEQ ID NO: 24. It is important to note that some species may favor one direction over the other. Moreover, many of these proteins are present in organisms that express unidirectional and bidirectional versions. The frdB, fumarate reductase iron-sulfur subunit, has an amino acid sequence as set forth in SEQ ID NO: 25. The g15 subunit has an amino acid sequence as set forth in SEQ ID NO: 26. The g13 subunit has an amino acid sequence as set forth in SEQ ID NO: 27.

Fumarate hydratase (EC 4.2.1.2) generates malate from fumarate and water. E. coli encode three distinct fumarate hydratases, though in preferred embodiments overexpression of one or more facilitates CO₂ fixation. The class I aerobic fumarate hydratase (fumA), locus CAA25204, has an amino acid sequence as set forth in SEQ ID NO: 28. The class I anaerobic fumarate hydratase (fumB), locus AAA23827, has an amino acid sequence as set forth in SEQ ID NO: 29. The class II fumarate hydratase (fumC), locus CAA27698, has an amino acid sequence as set forth in SEQ ID NO: 30.

L-malyl-CoA lyase (EC 4.2.1.2) generates acetyl-CoA and glyoxylate from L-malyl-CoA. An exemplary gene is mclA from Roseobacter denitrificans, locus NC_{—}008209.1, having an amino acid sequence as set forth in SEQ ID NO: 31.

The above enzyme activities, listed in this section, confer on E. coli the ability to synthesize an organic 2-carbon glyoxylate molecule from 2 molecules of CO₂. The stoichiometry of this reaction is 2 CO₂+3 ATP+3 NADPH Glyoxylate+2 ADP+2 Pi+AMP+PPi+3 NADP.

II. Enzymes for a Functional Reductive TCA Cycle

The following enzyme activities are expressed in E. coli to establish a functional reductive TCA cycle (FIG. 12). This pathway is employed by Chlorobium tepidum.

ATP-citrate lyase (EC. 2.3.3.8) generates acetyl-CoA, oxaloacetate, ADP, and Pi from citrate, ATP, and CoA. An exemplary ATP citrate lyase is the two subunit enzyme from Chlorobium tepidum, comprising ATP citrate lyase subunit 1, locus CY1089, having an amino acid sequence as set forth in SEQ ID NO: 32 and ATP citrate lyase subunit 2, locus CT1088, having an amino acid sequence as set forth in SEQ ID NO: 33.

Hydrogenobacter thermophilus employs an alternate pathway to generate oxaloacetate from citrate. In a first step, the 2 subunit citryl-CoA synthetase generates citryl-CoA from citrate, ATP, and CoA. The large subunit, ccsA, locus BAD17844 has an amino acid sequence as set forth in SEQ ID NO: 34. The small subunit, ccsB, locus BAD17846 has an amino acid sequence as set forth in SEQ ID NO: 35.

The Hydrogenobacter thermophilus citryl-CoA ligase (ccl), locus BAD 17841, generates oxaloacetate and acetyl-CoA from citryl-CoA has an amino acid sequence as set forth in SEQ ID NO: 36.

Malate dehydrogenase (EC 1.1.1.37) generates malate and NAD from oxaloacetate and NADH. An exemplary malate dehydrogenase from Chlorobium tepidum is locus CAA56810 having an amino acid sequence as set forth in SEQ ID NO: 37.

Fumarase (also known as fumarate hydratase) (EC 4.2.1.2) generates fumarate and water from malate. E. coli encodes 3 different fumarase genes, though in preferred embodiments it is useful to overexpress one or more to improve CO₂ fixation. An exemplary E. coli fumarase hydratase class I, (aerobic isozyme) is fumA, having an amino acid sequence as set forth in SEQ ID NO: 38. An exemplary E. coli fumarate hydratase class I (anaerobic isozyme) is fumB, having an amino acid sequence as set forth in SEQ ID NO: 39. An exemplary E. coli fumarate hydratase class II is fumC, having an amino acid sequence as set forth in SEQ ID NO: 40.

Succinate dehydrogenase (EC 1.3.99.1) generates succinate and FAD from fumarate and FADH₂. E. coli encodes a four-subunit succinate dehydrogenase complex (SdhCDAB), though in preferred embodiments, it is useful to overexpress these components to improve CO₂ fixation. These enzymes are also used in the 3-HPA pathway above, but in the reverse direction. It is important to note that some species may favor one direction or the other. Succinate dehydrogenase and fumarate reductase are reverse directions of the same enzymatic interconversion, succinate+FAD⁺ fumarate+FADH₂. In Escherichia coli, the forward and reverse reactions are catalyzed by distinct complexes: fumarate reductase operates under anaerobic conditions and succinate dehydrogenase operates under aerobic conditions. This group also includes a region of the B subunit of a cytosolic archaeal fumarate reductase. The SdhA flavoprotein subunit, locus NP_{—}415251 has an amino acid sequence as set forth in SEQ ID NO: 41. The SdhB iron-sulfur subunit, locus NP_{—}415252 has an amino acid sequence as set forth in SEQ ID NO: 42. The SdhC membrane anchor subunit, locus NP_{—}415249 has an amino acid sequence as set forth in SEQ ID NO: 43. The SdhD membrane anchor subunit, locus NP_{—}415250 has an amino acid sequence as set forth in SEQ ID NO: 44.

Acetyl-CoA:succinate CoA transferase (also known as succinyl-CoA synthetase) (EC 6.2.1.5) generates succinyl-CoA, ADP, and Pi from succinate, CoA, and ATP. E. coli encodes a heterotetramer of two alpha and beta subunits, though in preferred embodiments it is useful to overexpress these subunits to optimize CO₂ fixation. An exemplary E. coli succinyl-CoA synthetase subunit alpha is sucD, locus AAA23900 having an amino acid sequence as set forth in SEQ ID NO: 45. An exemplary E. coli succinyl-CoA synthetase subunit beta is sucC, locus AAA23899 having an amino acid sequence as set forth in SEQ ID NO: 46. Chlorobium tepidum sucC (AAM71626), with an amino acid sequence as set forth in SEQ ID NO: 105, and sucD (AAM71515), with an amino acid sequence as set forth in SEQ ID NO: 106, may also be used.

2-oxoketoglutarate synthase (also known as alpha-ketoglutarate synthase) (EC 1.2.7.3) generates alpha-ketoglutarate, CO₂, and oxidized ferredoxin from succinyl-CoA, CO₂, and reduced ferredoxin. An exemplary enzyme from Chlorobium limicola DSM 245 is a 4 subunit enzyme with accession numbers EAM42575 with an amino acid sequence as set forth in SEQ ID NO: 107; EAM42574 with an amino acid sequence as set forth in SEQ ID NO: 108; EAM42853 with an amino acid sequence as set forth in SEQ ID NO: 109; and EAM42852 with an amino acid sequence as set forth in SEQ ID NO: 110. This activity was functionally expressed in E. coli. Yun N R, Arai H, Ishii M, Igarashi Y. Biochem Biophys Res Communic (2001). The Genes for anabolic 2-oxoglutarate: Ferredoxin oxidoreductase from Hydrogenobacter thermophilus TK6. 282 (2): 589-594. There is another 5-subunit OGOR cluster in the same bacterium. Yun N R et al. Biochem Biophys Res Communic (2002). A novel five-subunit-type 2-oxoglutalate:ferredoxin oxidoreductases from Hydrogenobacter thermophilus TK-6. 292(1):280-6. The corresponding genes are for DABGE. An exemplary alpha-ketoglutarate synthase from Hydrogenobacter thermophilus is the heterodimeric enzyme that includes korA, locus AB046568:46-1869 with an amino acid sequence of: as set forth in SEQ ID NO: 47 and the korB locus AB046568:1883-2770 with an amino acid sequence of: as set forth in SEQ ID NO: 48.

Isocitrate dehydrogenase (EC 1.1.1.42) generates D-isocitrate and NADP+ from alpha-ketoglutarate, CO₂, and NADPH. An exemplary gene is the monomeric type idh from Chlorobium limicola, locus EAM42635 with an amino acid sequence of: as set forth in SEQ ID NO: 49. Another exemplary enzyme is that from Synechococcus sp WH 8102, icd, accession CAE06681, with an amino acid sequence as set forth in SEQ ID NO: 111.

In another embodiment, the NAD-dependent isocitrate dehydrogenase (EC 1.1.1.41) is expressed which generates isocitrate and NAD⁺ from alpha-ketoglutarate, CO₂, and NADH. An exemplary NAD-dependent enzyme is the two-subunit mitochondrial version from Saccharomyces cerevisiae. Subunit 1, idh1 locus YNL037C has an amino acid sequence as set forth in SEQ ID NO: 50. The second subunit, idh2, locus YOR136W has an amino acid sequence as set forth in SEQ ID NO: 51.

Aconitase (also known as aconitate hydratase or citrate hydrolyase) (EC 4.2.1.3) generates citrate from D-citrate via a cis-aconitate intermediate. E. coli encodes aconitate hydratase 1 and 2 (acnA and acnB), but in preferred embodiments it is useful to overexpress these enzymes to optimize CO₂ fixation. An exemplary aconitate hydrase 1 is E. coli acnA, locus b1276, having an amino acid sequence as set forth in SEQ ID NO: 52. An exemplary E. coli aconitate hydratase 2 is acnB, locus b0118, having an amino acid sequence as set forth in SEQ ID NO: 53.

Pyruvate synthase (also known as pyruvate:ferredoxin oxidoreductase) (EC 1.2.7.1) generates pyruvate, CoA, and an oxidized ferrodoxin from acetyl-CoA, CO₂, and a reduced ferredoxin. An exemplary pyruvate synthase is the tetrameric enzyme porABCD from Clostridium tetani E88, whereby subunit porA, locus AA036986 has an amino acid sequence as set forth in SEQ ID NO: 54; subunit porB, locus AA036985 has an amino acid sequence as set forth in SEQ ID NO: 55; subunit porC, locus AA036988 has an amino acid sequence as set forth in SEQ ID NO: 56; and subunit porD, locus AA036987 has an amino acid sequence as set forth in SEQ ID NO: 57.

Phosphoenolpyruvate synthase (also known as PEP synthase, pyruvate, water dikinase) (EC 2.7.9.2) generates phosphoenolpyruvate, AMP, and Pi from pyruvate, ATP, and water. E. coli encodes an exemplary PEP synthase, ppsA, though in preferred embodiments it is useful to overexpress ppsA to optimize CO₂ fixation. The E. coli ppsA enzyme, locus AAA24319 has an amino acid sequence as set forth in SEQ ID NO: 58. The corresponding enzyme from Aquifex aeolicus VF5 ppsA, locus AAC07865, with an amino acid sequence as set forth in SEQ ID NO: 112, may also be used.

Phosphoenolpyruvate carboxylase (also known as PEP carboxylase PEPCase, PEPC) (EC 4.1.1.31) generates oxaloacetate and Pi from phosphoenolpyruvate, water, and CO₂. E. coli encodes an exemplary PEP carboxylase, ppC, though in preferred embodiments it is useful to overexpress ppC to optimize CO₂ fixation. The E. coli ppC enzyme, locus CAA29332 has an amino acid sequence as set forth in SEQ ID NO: 59.

The above enzymes, described in this section, confer upon E. coli the ability to synthesize an organic 2-carbon acetyl-CoA molecule from 2 molecules of CO₂. The stoichiometry of this reaction is 2 CO₂+2 ATP+3 NADH+1 FADH₂+CoASH acetyl-CoA+2 ADP+2 Pi+AMP+PPi+FAD+3 NAD⁺.

III. Enzymes for a Functional Woods-Ljungdahl Cycle

The following enzyme activities are expressed in E. coli to establish a functional Woods-Ljungdahl pathway (FIG. 11). This pathway is employed by Moorella thermoacetica (previously known as Clostridium thermoaceticum), Methanobacterium thermoautrophicum, and Desulfobacterium autotrophicum.

NADP-dependent formate dehydrogenase (EC 1.2.1.4.3) generates formate and NADP⁺ from CO₂ and NADPH. An exemplary NADP-dependent formate dehydrogenase is the two-subunit Mt-fdhA/B enzyme from Moorella thermoacetica (previously known as Clostridium thermoaceticum) which contains Mt-fdhA, locus AAB18330, having an amino acid sequence as set forth in SEQ ID NO: 60 and the beta subunit, Mt-fdhB, locus AAB18329, having an amino acid sequence as set forth in SEQ ID NO: 61.

Formate tetrahydrofolate ligase (EC 6.3.4.3) generates 10-formyltetrahydrofolate, ADP, and Pi from formate, ATP, and tetrahydrofolate. An exemplary formate tetrahydrofolate ligase is from Clostridium acidi-urici, locus M21507, having an amino acid sequence as set forth in SEQ ID NO: 62. Alternate sources for this enzyme activity include locus AAB49329 from Streptococcus mutans (Swiss-Prot entry Q59925), with an amino acid sequence as set forth in SEQ ID NO: 113, or the protein with Swiss-Prot entry Q8XHL4 from Clostridium perfringens encoded by the locus BA000016, with an amino acid sequence as set forth in SEQ ID NO: 114.

Methenyltetrahydrofolate cyclohydrolase (also known as 5,10-methylenetetrahydrofolate dehydrogenase) (EC 3.5.4.9 and 1.5.1.5) generates 5,10-methylene-THF, water, and NADP from 10-formyltetrahydrofolate and NADPH via a 5,10-methyenyltetrahydrofolate intermediate. E. coli encodes a bifunctional methenyltetrahydrofolate cyclohydrolase/dehydrogenase, folD, though in preferred embodiments it is useful to overexpress this gene to optimize CO₂ fixation. The E. coli enzyme, locus AAA23803, has an amino acid sequence as set forth in SEQ ID NO: 63. Alternate sources for this enzyme activity include locus ABC 19825 (folD) from Moorella thermoacetica, with an amino acid sequence as set forth in SEQ ID NO: 115; locus AAO36126 from Clostridium tetani, with an amino acid sequence as set forth in SEQ ID NO: 116; and locus BAB81529 from Clostridium perfringens, with an amino acid sequence as set forth in SEQ ID NO: 117. All are bifunctional folD enzymes.

Methylene tetrahydrofolate reductase (EC 1.5.1.20) generates 5-methyltetrahydrofolate and NADP⁺ from 5,10-methylene-trahydrofolate and NADPH. E. coli encodes an exemplary methylene tetrahydrofolate reductase, metF, though in preferred embodiments it is useful to overexpress this gene to optimize CO₂ fixation. The E. coli enzyme, locus CAA24747, has an amino acid sequence as set forth in SEQ ID NO: 64. Alternative sources for this enzyme activity include bifunctional folD enzymes such as locus ABC 19825 (folD) from Moorella thermoacetica, with an amino acid sequence as set forth in SEQ ID NO: 115; locus AA036126 from Clostridium tetani, with an amino acid sequence as set forth in SEQ ID NO: 116; and locus BAB81529 from Clostridium perfringens, with an amino acid sequence as set forth in SEQ ID NO: 117; locus AAC23094 from Haemophilus influenzae, with an amino acid sequence as set forth in SEQ ID NO: 118; and locus CAA30531 from Salmonella typhimurium, with an amino acid sequence as set forth in SEQ ID NO: 119.

5-methyltetrahydrofolate corrinoid/iron sulfur protein methyltransferase generates tetrahydrofolate and a methylated corrinoid Fe—S protein from 5-methyl-tetrahydrofolate and a corrinoid Fe—S protein. An exemplary gene, acsE, is encoded by locus AAA53548 in Moorella thermoacetica and has an amino acid sequence as set forth in SEQ ID NO: 65. This activity has been functionally expressed in E. coli (Roberts D L, Zhao S, Doukov T, and Ragsdale S. The reductive acetyl-CoA Pathway: Sequence and heterologous expression of active methyltetrahydrofolate:corrinoid/Urib-sulfur protein methyltransferase from Clostridium thermoaceticum. J. Bacteriol (1994). 176(19):6127-30). Another source for this activity is encoded by the acsE gene from Carboxydothermus hydrogenoformas locus CP000141, with an amino acid sequence as set forth in SEQ ID NO: 120.

Carbon monoxide dehydrogenase/acetyl-CoA synthase (EC 1.2.7.4/1.2.99.2 and 2.3.1.169) is a bifunctional two-subunit enzyme which generates acetyl-CoA, water, oxidized ferredoxin, and a corrinoid protein from CO₂, reduced ferredoxin, and a methylated corrinoid protein. An exemplary carbon monoxide dehydrogenase enzyme, subunit beta, is encoded by locus AAA23228 from Moorella thermoacetica and has an amino acid sequence as set forth in SEQ ID NO: 66. Another exemplary source of this activity is encoded by the acsB gene, locus CHY_{—}1222 from Carboxydothermus hydrogenoformase with protein accession YP_{—}360060, with an amino acid sequence as set forth in SEQ ID NO: 121. An exemplary acetyl-CoA synthase, subunit alpha, is locus AAA23229 from Moorella thermoacetica and has an amino acid sequence as set forth in SEQ ID NO: 67.

The above enzymes, described in this section, confer upon E. coli the ability to synthesize an organic 2-carbon acetyl-CoA molecule from 2 molecules of CO₂. The stoichiometry of this reaction is 2 CO₂+1 ATP+2 NADPH+2 reduced ferredoxins+coenzyme A acetyl-CoA+2H₂O+ADP+Pi+2 NADP⁺+2 oxidized ferredoxins.

IV. Additional Carbon Fixation Pathway Genes

In addition to the enzymes above, cells may be engineered to fix carbon by incorporating wild-type or codon optimized nucleic acids expressing Salinibacter fructose-bisphosphate aldolase, Synechococcus sp. 7002 fructose-bisphosphate aldolase (class I), Synechococcus elongatus PCC 7942 sedoheptulose-1,7-bisphosphatase, and/or T. elongatus BP-1 sedoheptulose-1,7-bisphosphatase (see, e.g., SEQ ID NOs 261-270).

### Example 7

### Engineering the Glyoxylate Shunt

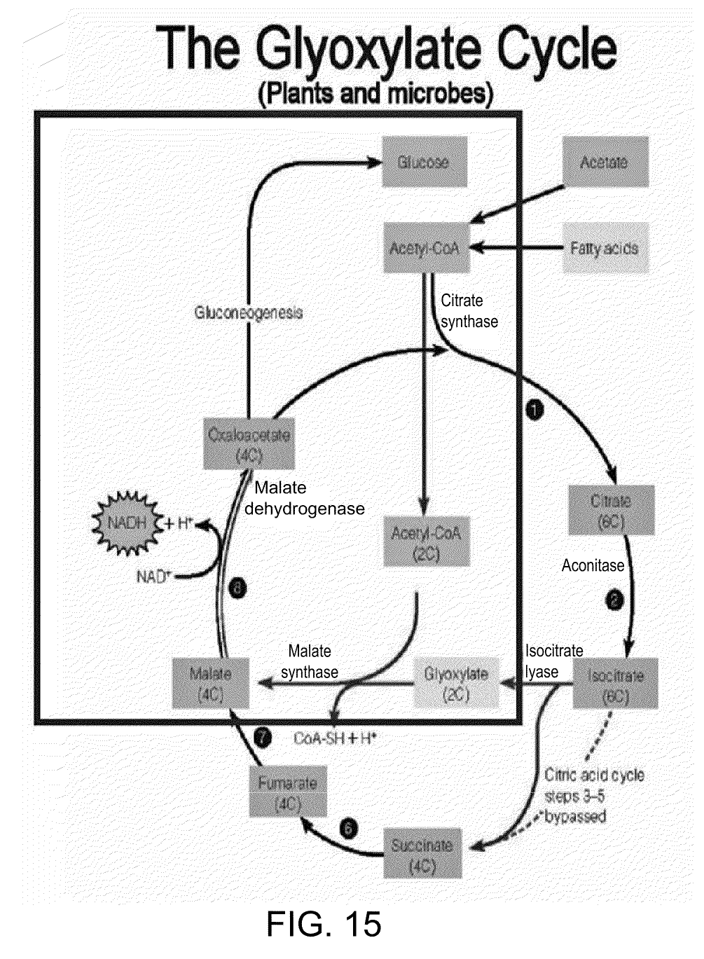

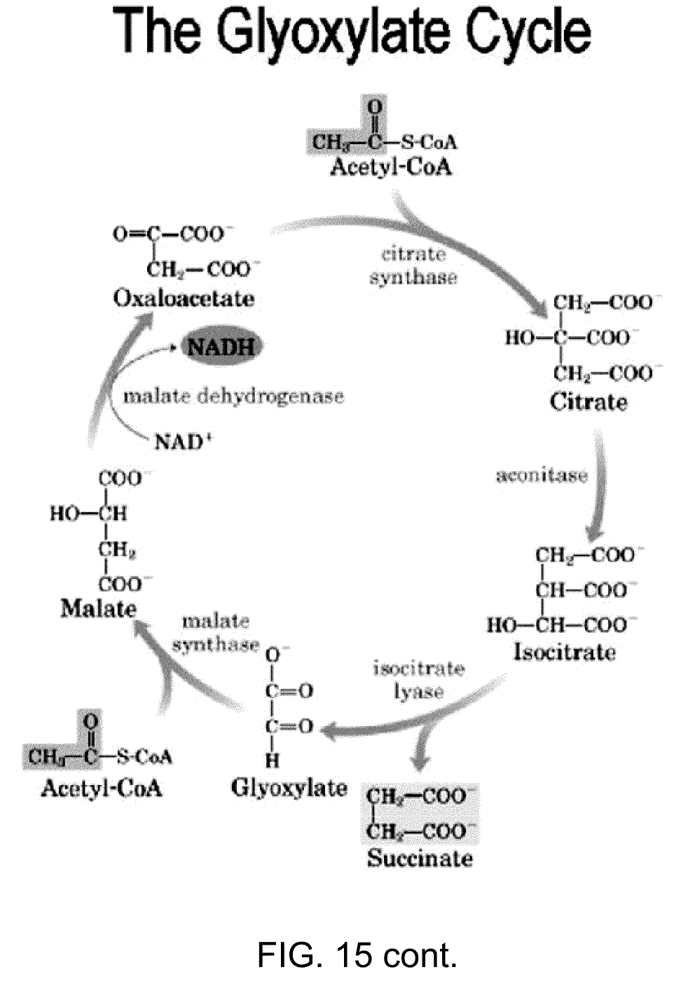

The enzymes described earlier provide pathways to assimilate CO₂ into the 2-carbon acetyl-CoA (reductive TCA and Woods-Ljungdahl pathways) or glyoxylate (3-HPA pathway). Combinations of these (preferentially the 3-HPA cycle and the reductive TCA cycle) are also engineered in special cases. In this scenario, the outputs of the CO₂ fixation reactions (acetyl-CoA and glyoxylate) are utilized as inputs for the glyoxylate cycle (FIG. 15), which combines acetyl-CoA and glyoxylate into 4-carbon oxaloacetate (via a 4-carbon malate intermediate) [Chung T, Klumpp D J, Laporte D C. J Bacteriol (1988). “Glyoxylate bypass operon of Escherichia coli: cloning and determination of the functional map.” 170(1):386-92.]

Three key enzymes are involved in the Escherichia coli glyoxylate shunt pathway. In preferred embodiments, all are overexpressed to maximize CO₂ fixation.

Malate synthase (EC 2.3.3.9) generates malate and coenzyme A from acetyl-CoA, water, and glyoxylate. An exemplary enzyme is encoded by E. coli locus JW3974 (aceB) with an amino acid sequence as set forth in SEQ ID NO: 68. Another exemplary activity is provided by an alternate malate synthase enzyme E. coli encodes, the JW2943 locus malate synthase G (glcB), having an amino acid sequence as set forth in SEQ ID NO: 69.

Isocitrate lyase (EC 4.1.3.1) generates glyoxylate and succinate from isocitrate. An exemplary enzyme is that encoded by E. coli locus JW3975 (aceA) having an amino acid sequence as set forth in SEQ ID NO: 70. Although isocitrate lyase is critical for E. coli's endogenous glyoxylate bypass, this activity does not need to be overexpressed in practicing the instant invention. The enzyme's main purpose in the pathway is to generate glyoxylate, which can instead be supplied via the engineered 3-HPA pathway.

Malate dehydrogenase (EC 1.1.1.37) generates oxaloacetate and NADH from malate and NAD⁺. An exemplary enzyme is that encoded by E. coli locus JW3205 (mdh) with an amino acid sequence as set forth in SEQ ID NO: 71.

### Example 8

### Engineering Gluconeogenesis

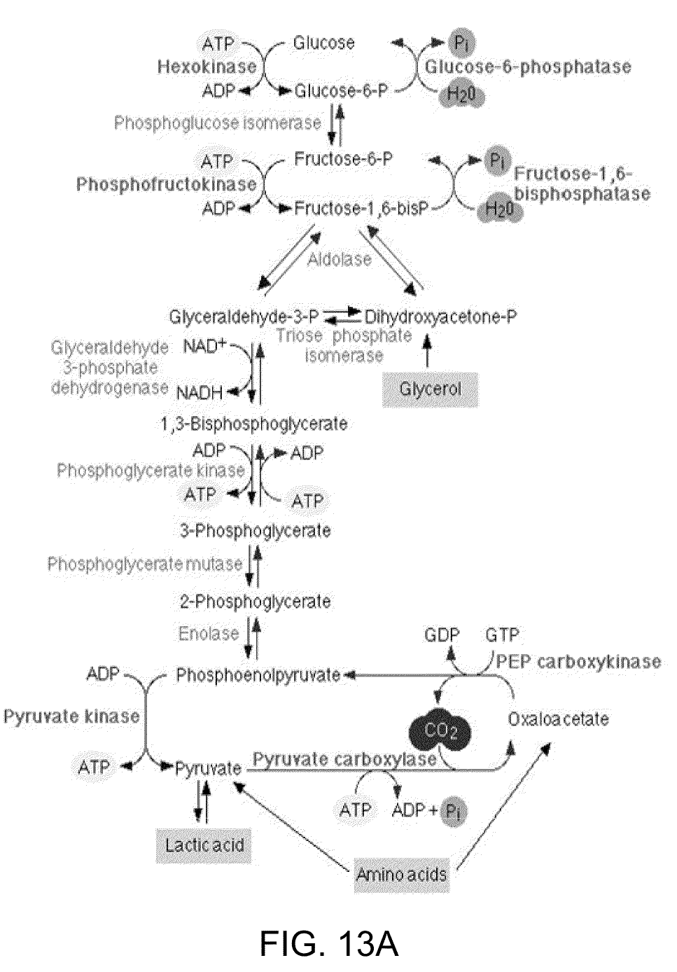

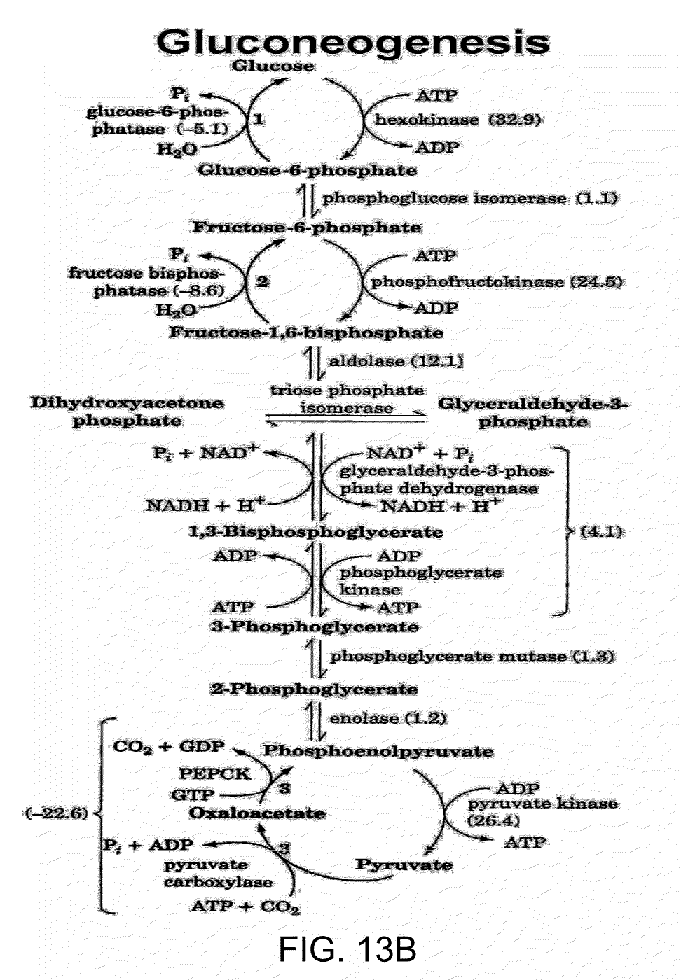

Gluconeogenesis is the process by which organisms generate glucose from non-sugar carbon substrates, including pyruvate, lactate, glycerol, and glucogenic amino acids. Most steps of glycolysis are bidirectional, with three exceptions (reviewed in Hers H G, Hue, L. Ann Rev. Biochem (1983). “Gluconeogenesis and related aspects of glycolysis.” 52:617-53). These enzyme activities are expressed to enable gluconeogenesis in E. coli (FIG. 13).

I. Conversion of Pyruvate to Phosphoenolpyruvate

Conversion of pyruvate to phosphoenolpyruvate requires two enzymatic activities as follows.

Pyruvate carboxylase (EC 6.4.4.1) generates oxaloacetate, ADP, and Pi from pyruvate, ATP, and CO₂. An exemplary pyruvate carboxylase is encoded by the YGL062W locus from Saccharomyces cerevisiae, pyc1, and has an amino acid sequence as set forth in SEQ ID NO: 72.

Phosphoenolpyruvate carboxykinase (EC 4.1.1.49) generates phosphoenolpyurate, ADP, Pi, and CO₂ from oxaloacetate and ATP. An exemplary phosphoenolpyruvate carboxykinase is encoded by E. coli locus JW3366, pckA, and has an amino acid sequence as set forth in SEQ ID NO: 73.

II. Conversion of Fructose 1,6-bisphosphate to Fructose-6-phosphate

Conversion of fructose 1,6-bisphosphate to fructose-6-phosphate requires fructose-1,6-bisphosphatase (EC 3.1.3.11), which generates fructose-6-phosphate and Pi from fructose-1,6-bisphosphate and water. An exemplary fructose-1,6-bisphosphatase is encoded by E. coli locus JW4191, fbp, and has an amino acid sequence as set forth in SEQ ID NO: 74.

III. Conversion of Glucose-6-phosphate to Glucose

Conversion of glucose-6-phosphate to glucose requires glucose-6-phosphatase (EC 3.1.3.68), which generates glucose and Pi from glucose-6-phosphate and water. An exemplary glucose-6-phosphatase is encoded by the Saccharomyces cerevisiae YHR044C locus, dog1, and has an amino acid sequence as set forth in SEQ ID NO: 75. Another exemplary glucose-6-phosphatase activity is encoded by Saccharomyces cerevisiae YHR043C locus, dog2, and has an amino acid sequence as set forth in SEQ ID NO: 76.

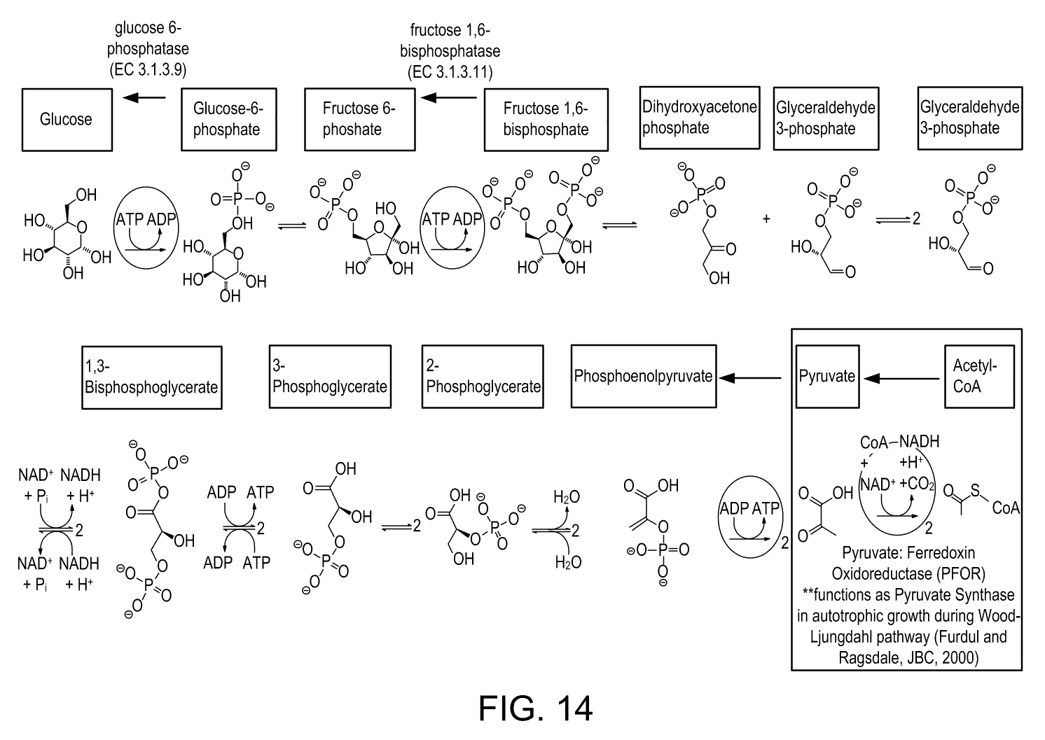

Oxaloacetate, the starting material for gluconeogenesis, is generated either via the glyoxylate shunt (leveraging inputs from the reductive TCA or Woods-Ljungdahl pathways and the 3-HPA pathway) or via the carboxylation of pyruvate. In the absence of the glyoxylate shunt, the pyruvate synthase activity of pyruvate ferredoxin:oxidoreductase (EC 1.2.7.1) can generate pyruvate, CoA, and oxidized ferredoxin from acetyl-CoA, CO₂, and reduced ferredoxin [Furdui C and Ragsdale S W. J. Biol. Chem. (2000). “The role of pyruvate ferredoxin oxidoreductase in pyruvate synthesis during autotrophic growth by the Woods-Ljungdahl pathway.” 275(37): 28494-99] (FIG. 14). An exemplary pyruvate ferredoxin oxidoreductase with pyruvate synthase activity is encoded by locus Moth-0064 from Moorella thermoaceticum, and has an amino acid sequence as set forth in SEQ ID NO: 77.

### Example 9

### Engineering Reducing Power

The above CO₂-fixation pathways require reducing power, primarily in the form of NADH and NADPH. Maintaining an appropriately-balanced supply of reduced NAD⁺ (NADH) and NADP⁺ (NADPH) is important to maximize carbon assimilation, and thus growth rate, of engineered E. coli.

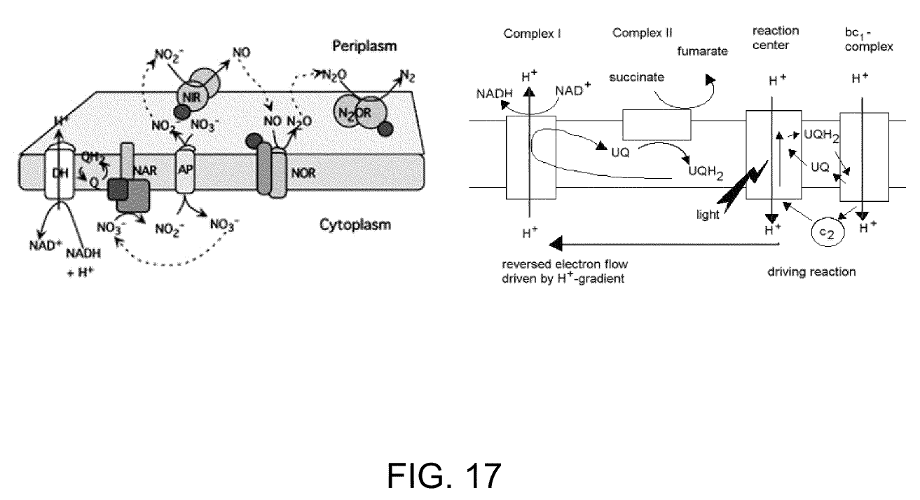

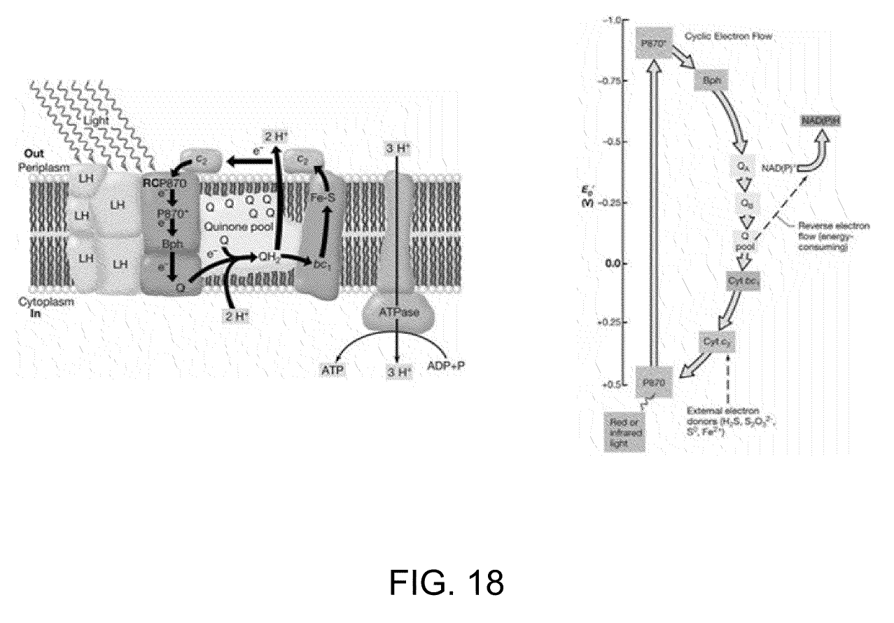

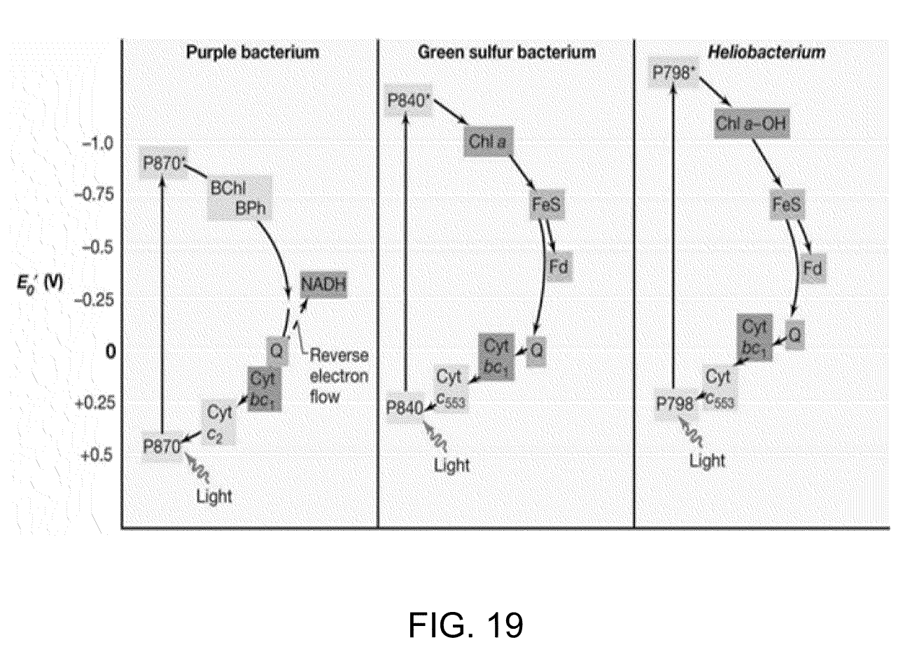

Table 1 lists candidate genes for overexpression in the reducing power module together with information on associated pathways, Enzyme Commission (EC) Numbers, exemplary gene names, source organism, GenBank accession numbers, and homologs from alternate sources. FIG. 17, FIG. 18, and FIG. 19 show possible mechanisms to generate reducing power.

I. NADH

As described in the section on engineering light capture, disruption of endogenous nuo and/or ndh loci significantly increases the intracellular ratio of NADH:NAD⁺. When NADH levels remain suboptimal, a plurality of additional methods is employed including overexpression of the following genes.

NAD⁺-dependent isocitrate dehydrogenase (EC 1.1.1.41) generates 2-oxoglutarate, CO₂, and NADH from isocitrate and NAD⁺. Of note, most bacterial isocitrate dehydrogenases are NADP⁺-dependent (EC 1.1.1.42). An exemplary NAD⁺-dependent isocitrate dehydrogenase is the octameric Saccharomyces cerevisiae enzyme comprising locus YNL037C, idh1, encoding a protein having the amino acid sequence as set forth in SEQ ID NO: 78 and locus YOR136W, idh2, encoding a protein having an amino acid sequence as set forth in SEQ ID NO: 79.

Malate dehydrogenase (EC 1.1.1.37) generates oxaloacetate and NADH from malate and NAD⁺. As described above, this enzyme is overexpressed in embodiments leveraging the glyoxylate shunt. Irrespective of the employment of the glyoxylate shunt, overexpression of NAD-dependent malate dehydrogenase can be employed to increase NADH pools. An exemplary enzyme is encoded by E. coli locus JW3205 (mdh) and has an amino acid sequence as set forth in SEQ ID NO: 80.

The NADH:ubiquinone oxidoreductase from Rhodobacter capsulatus, is unique in its ability to reverse electron flow between the quinone pool and NAD⁺ [Dupuis A, Peinnequin A, Darrouzet E, Lunardi J. FEMS Microbiol Lett (1997). “Genetic disruption of the respiratory NADH-ubiquinone reductase of Rhodobacter capsulatus leads to an unexpected photosynthesis-negative phenotype.” 149:107-114; Dupuis A, Darrouzet E, Duborjal H, Pierrard B, Chevallet M, van Belzen R, Albracht S P J, Lunardi J. Mol. Microbiol. (1998). “Distal genes of the nuo-operon of Rhodobacter capsulatus equivalent to the mitochondrial ND subunits are all essential for the biogenesis of the respiratory NADH-ubiquinone oxidoreductase. 28:531-541]. E. coli nuo can be knocked out as a means to increase NADH amounts. The Rhodobacter Nuo operon, encoding the Nuo Complex I, can be reconstituted to generate additional NADH by reverse electron flow.

The Rhodobacter capsulatus nuo operon, locus AF029365, consisting of the 14 nuo genes nuoA-N (and 7 ORFs of unknown function) can be expressed to enable reverse electron flow and NADH-generation in E. coli. The operon encodes NuoA, accession AAC24985.1, having an amino acid sequence as set forth in SEQ ID NO: 81; NuoB, accession AAC24986.1, having an amino acid sequence as set forth in SEQ ID NO: 82; NuoC, accession AAC24987.1, having an amino acid sequence as set forth in SEQ ID NO: 83; NuoD, accession AAC24988.1, having an amino acid sequence as set forth in SEQ ID NO: 84; NuoE, accession AAC24989.1, having an amino acid sequence as set forth in SEQ ID NO: 85; NuoF, accession AAC24991.1, having an amino acid sequence as set forth in SEQ ID NO: 86; NuoG, accession AAC24995.1 has an amino acid sequence as set forth in SEQ ID NO: 87; NuoH, accession AAC24997.1, having an amino acid sequence as set forth in SEQ ID NO: 88; NuoI, accession AAC24999.1, having an amino acid sequence as set forth in SEQ ID NO: 89; NuoJ, accession AAC25001.1, having an amino acid sequence as set forth in SEQ ID NO: 90; NuoK, accession AAC25002.1, having an amino acid sequence as set forth in SEQ ID NO: 91; NuoL, accession AAC25003.1, having an amino acid sequence as set forth in SEQ ID NO: 92; NuoM, accession AAC25004.1, having an amino acid sequence as set forth in SEQ ID NO: 93; and NuoN, accession AAC25005.1, having an amino acid sequence as set forth in SEQ ID NO: 94.

Expression of pyridine nucleotide transhydrogenase (EC 1.6.1.1) generates NADH and NADP⁺ from NADPH and NAD⁺. An exemplary enzyme is the E. coli soluble pyridine nucleotide transhydrogenase, encoded by sthA (also known as udhA), locus JW551, having an amino acid sequence as set forth in SEQ ID NO: 100. An alternate exemplary enzyme is the membrane bound E. coli pyridine nucleotide transhydrogenase, encoded by the multisubunit of NAD(P) transhydrogenase subunit alpha, encoded by pntA, locus JW1595, having an amino acid sequence as set forth in SEQ ID NO: 101 and NADP transhydrogenase subunit beta, encoded by pntB, locus JW1594, with an amino acid sequence as set forth in SEQ ID NO: 102.

II. NADPH

NADPH serves as an electron donor in reductive (especially fatty acid) biosynthesis. Three parallel methods are used, singly or in combination, to maintain sufficient NADPH levels for photoautotrophy. Methods 1 and 2 are described in WO2001/007626, Methods for producing L-amino acids by increasing cellular NADPH. Method 3 is described in U.S. Pub. No. 2005/0196866, Increasing intracellular NADPH availability in E. coli.

A. Increasing the Flux Through the Pentose Phosphate Pathway

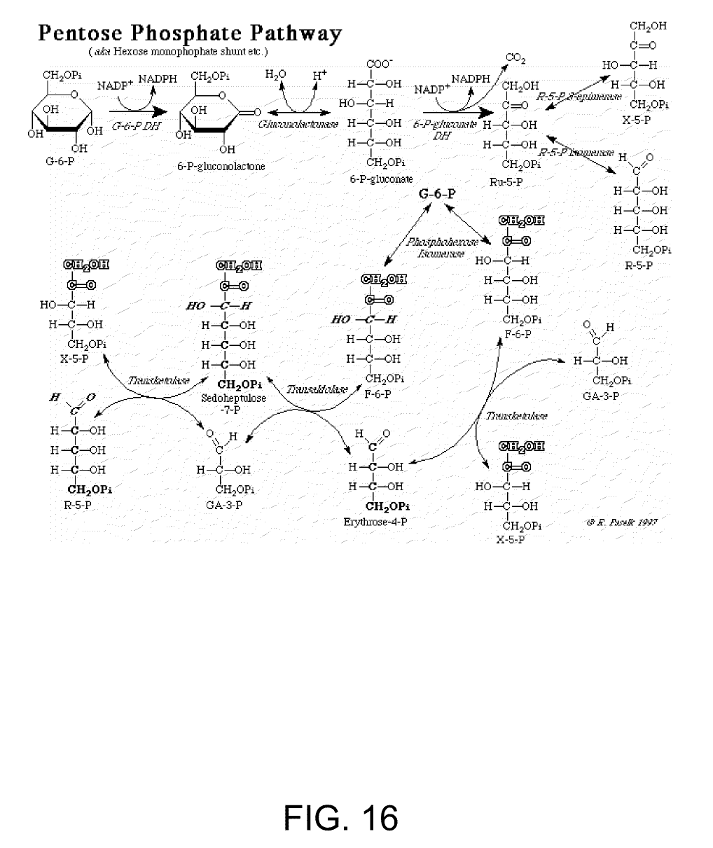

Increasing the flux through the Pentose Phosphate Pathway generates 2 molecules of NADPH per molecule of glucose (FIG. 16).

The inactivation of the E. coli phosphoglucose isomerase, pgi, locus JW3985, is known to force glucose through the pentose phosphate pathway. This therefore provides one approach for increasing intracellular NADPH pools [Kabir, M M. Shimizu, K. Appl. Microbiol. Biotechnol. (2003):Fermentation characteristics and protein expression patterns in a recombinant Escherichia coli mutant lacking phosphoglucose isomerase for poly(3-hydroxybutyrate) production.” 62:244-255; Kabir M M, Shimizu K. J. Biotechnol (2003). “Gene expression patterns for metabolic pathway in pgi knockout Escherichia coli with and without phb genes based on RT-PCR” 105(1-2):11-31.]

Overexpression of glucose-6-phosphate dehydrogenase (EC 1.1.1.49), which generates NADPH and 6-phospho-gluconolactone from glucose-6-phosphate and NADP⁺, provides another way to increase NADPH levels. An exemplary enzyme is that encoded by E. coli glucose-6-phosphate dehydrogenase, zwf locus JW1841 and having an amino acid sequence as set forth in SEQ ID NO: 95.

Overexpression of 6-phosphogluconolactonase (EC 3.1.1.31), which generates 6-phosphogluconate from 6-phosphoglucolactone and water, provides another approach for increasing flux through the pentose phosphate pathway. An exemplary enzyme is that encoded by the E. coli 6-phosphogluconolactonase, pgl, locus JW0750, having an amino acid sequence as set forth in SEQ ID NO: 96.

Overexpression of 6-phosphogluconate dehydrogenase (EC 1.1.1.44) generates ribose-5-phosphate, CO₂, and NADPH from 6-phosphogluconate and NADP⁺. This also can be used to increase NADPH levels by increasing flux through the pentose phosphate pathway. An exemplary enzyme is the encoded by E. coli 6-phosphogluconate dehydrogenase, gnd, locus JW2011, having an amino acid sequence as set forth in SEQ ID NO: 97.

B. Expression of NADP⁺-Dependent Enzymes

NADP⁺-dependent enzymes can be expressed in lieu of or in addition to NAD-dependent enzymes.

Overexpression of isocitrate dehydrogenase (EC 1.1.1.42) generates 2-oxoglutarate, CO₂, and NADPH from isocitrate and NADP⁺. An exemplary enzyme is encoded by the E. coli isocitrate dehydrogenase, icd, locus JW1122, and has an amino acid sequence as set forth in SEQ ID NO: 98.

Overexpression of malic enzyme (EC 1.1.1.40) generates pyruvate, CO₂, and NADPH from malate and NADP⁺. An exemplary NADP-dependent enzyme is the E. coli malic enzyme, encoded by maeB, locus JW2447, having an amino acid sequence as set forth in SEQ ID NO: 99.

C. Expression of Pyridine Nucleotide Transhydrogenase

Expression of pyridine nucleotide transhydrogenase (EC 1.6.1.1) generates NADPH and NAD⁺ from NADH and NADP⁺. An exemplary enzyme is the E. coli soluble pyridine nucleotide transhydrogenase, encoded by sthA (also known as udhA), locus JW551, having an amino acid sequence as set forth in SEQ ID NO: 100. An alternate exemplary enzyme is the membrane bound E. coli pyridine nucleotide transhydrogenase, encoded by the multisubunit of NAD(P) transhydrogenase subunit alpha, encoded by pntA, locus JW1595, having an amino acid sequence as set forth in SEQ ID NO: 101 and NADP transhydrogenase subunit beta, encoded by pntB, locus JW1594, with an amino acid sequence as set forth in SEQ ID NO: 102.

### Example 10

### Engineering Carbon Acetyl-coA Flux

In some embodiments of the present invention, methods may be employed to overexpress pantothenate kinase, encoded by panK, locus AAC76952 and/or pyruvate dehydrogenase, encoded by aceE, locus AAC73225 and aceF, locus NP_{—}414657 as a means of raising acetyl-CoA levels and, optionally, increasing overall fatty acid production [Vadali R V, Bennett G N, San K Y. Applicability of CoA/acetyl-CoA manipulation system to enhance isoamyl acetate production in Escherichia coli. Metab Eng. 2004 October; 6(4):294-9]. Additional approaches may include the downregulation, inhibition, or knocking out of acyl coenzyme A dehydrogenase, encoded by fadE, locus NP_{—}414756, biosynthetic glycerol 3-phosphate dehydrogenase, GpsA, locus BAE77684, lactate dehydrogenase, encoded by ldhA. Locus NP_{—}415898, formate acetyltransferase 1, encoded by pflb, locus NP_{—}415-423, alcohol dehydrogenase, encoded by adhE, locus NP_{—}415757. phosphotransacetylase, encoded by PTA, locus NP_{—}416800, pyruvate oxidase, encoded by poxB, locus AAB31180, and acetate kinase, encoded by ackA and ackB, locus NP_{—}416799. Additional methods include overexpressing accABCD (encoding acetyl co-A carboxylase), aceEF (encoding the E1p dehydrogase component and the E2p dihydrolipoamide acyltransferase component of the pyruvate and 2-oxoglutarate dehydrogenase complexes), fabH/fabD/fabG/acpP/fabF (encoding FAS), fatty-acyl-coA reductases and aldehyde decarbonylases as well as limiting the cellular supply of glycerol (to less than 1% w/v of the medium). In some embodiments, such methods may increase expression of a heterologous DNA sequence in the host cell by 2-fold, as compared with the wild-type host cell. In other embodiments, such methods may increase expression of a heterologous DNA sequence in the host cell by 5-fold. In further embodiments, such methods may increase expression of a heterologous DNA sequence in the host cell by 10-fold. In other embodiments, such methods may increase expression of a heterologous DNA sequence in the host cell by 100-fold. In further embodiments, such methods may increase expression of a heterologous DNA sequence in the host cell by 1000-fold.

In other embodiments, methods may be employed to increase or improve fatty acid production in a synthetophototrophic cell. Increased flux through acetyl-CoA and malonyl-CoA maximizes hydrocarbon and/or hydrocarbon precursor production.

A series of modifications are carried out in order to obtain acetyl CoA/malonyl CoA/fatty acid overproducers. For example, to increase flux through acetyl-CoA, a biosynthetic pathway is introduced via a plasmid, cosmid, fosmid, or BAC that encodes PDH, PanK, aceEF, (encoding the E1p dehydrogenase component and the E2p dihydrolipoamide acyltransferase component of the pyruvate and 2-oxoglutarate dehydrogenase complexes), fabH/fabD/fabG/acpP/fabF (encoding FAS), and potentially additional DNA encoding fatty-acyl-coA reductases and aldehyde decarbonylases, each under the control of a constitutive promoter, from Codon Devices (Cambridge, Mass.). The sequences of all these genes can be found at http://www.ncbi.nlm.nih.gov/entrez/query.fcgi?CMD=search&DB=nucleotide). Subsequently, FadE, GpsA, LdhA, pflb, adhE, PTA, poxB, ackA, and/or ackB may be knocked out of the engineered microbe by transformation with plasmids containing null mutations of the corresponding genes or other methods known to those skilled in the art. The sequences of all these genes can be found at http://www.ncbi.nlm.nih.gov/entrez/query.fcgi?CMD=search&DB=nucleotide).

The resulting synthetophototrophic organisms may be grown in the presence of light and carbon dioxide under conditions to sufficient to synthesize hydrocarbon products or precursors. As such, these microorganisms will have increased acetyl CoA production levels. Malonyl CoA overproduction may be effected by engineering the microorganism as described above, with DNA encoding accABCD (acetyl CoA carboxylase) included in the plasmid synthesized de novo. Fatty acid overproduction may be achieved by further including DNA encoding lipase in the plasmid synthesized de novo. For various length precursors, specific other genes may be knocked out. For C18, AF503757 (which uses C20-ACP) may be knocked out and POADA1 (which uses C16-ACP) may be included in the synthesized plasmid. For C16, AF503757 and POADA1 may be knocked out and Q39473 (which uses C14-ACP) may be included in the synthesized plasmid. For C14, Q39473, AF503757 and POADA1 may be knocked out, and AAA34215 (which uses C12-ACP) may be included in the synthesized plasmid. Acetyl CoA, malonyl CoA, and/or fatty acid overproduction can be verified by using radioactive precursors, HPLC, and GC-MS subsequent to cell lysis.

Knocking out lactate and acetate production in Clostridium thermocellum has been demonstrated to increase the total amount of ethanol production without reducing the total carbon progressing through the common biosynthetic pathway (Shaw, J., et al., “Metabolic Engineering of the Xylose Utilizing Thermophile Thermoanaerobacterium saccharolyticum JW/SL-YS485 for Ethanol Production.” presented at AICHE Annual Meeting).

In some embodiments Acetyl-CoA carboxylase (ACC) or Malonyl-CoA decarboxylase may be overexpressed in order to increase the intracellular concentration thereof by at least 2-fold. In a preferred embodiment, Acetyl-CoA carboxylase (ACC) or Malonyl-CoA decarboxylase may be overexpressed in order to increase the intracellular concentration thereof by at least 5-fold. In a more preferred embodiment, Acetyl-CoA carboxylase (ACC) or Malonyl-CoA decarboxylase may be overexpressed so as to increase the intracellular concentration thereof by at least 10-fold.

In some embodiments, the intracellular concentration (e.g., the concentration of the intermediate in the genetically modified host cell) of the biosynthetic pathway intermediate may be increased to further boost the yield of the final product. The intracellular concentration of the intermediate can be increased in a number of ways, including, but not limited to, increasing the concentration in the culture medium of a substrate for a biosynthetic pathway; increasing the catalytic activity of an enzyme that is active in the biosynthetic pathway; increasing the intracellular amount of a substrate (e.g., a primary substrate) for an enzyme that is active in the biosynthetic pathway; and the like.

Table 4, which follows, briefly describes each of the sequences in the formal sequence listing filed with this application.

TABLE 4

| SEQ ID NO: | Description of Sequence |
|---|---|
| 1 | Amino acid sequence of a proteorhodopsin (locus ABL60988) |
| 2 | Amino acid sequence of a bacteriorhodopsin (locus NP_280292) |
| 3 | Amino acid sequence of a deltarhodopsin (locus AB009620) |
| 4 | Amino acid sequence of a xanthorhodopsin (locus ABC44767) |
| 5 | Amino acid sequence of a isopentenyl-diphosphate delta-isomerase (Idi) (locus |
| | ABL60982) |
| 6 | Amino acid sequence of a 15,15′-beta-carotene dioxygenase (Blh) (locus ABL60983) |
| 7 | Amino acid sequence of a lycopene cyclase (CrtY) (locus ABL60984) |
| 8 | Amino acid sequence of a phytoene synthase (CrtB) (EC 2.5.1.32) (locus ABL60985) |
| 9 | Amino acid sequence of a phytoene dehydrogenase (CrtI) (locus ABL60986) |
| 10 | Amino acid sequence of a geranylgeranyl pyrophosphate synthetase (CrtE) (locus |
| | ABL60987) |
| 11 | Amino acid sequence of a beta-carotene ketolase (CrtO) (locus SRU_1502) |
| 12 | Amino acid sequence of a acetyl-CoA carboxylase subunit alpha (AccA) (locus |
| | AAA70370) |
| 13 | Amino acid sequence of a acetyl-CoA carboxylase subunit beta (accD) (locus AAA23807) |
| 14 | Amino acid sequence of a biotin-carboxyl carrier protein (AccB) (locus ECOACOAC) |
| 15 | Amino acid sequence of a biotin carboxylase (AccC) (locus AAA23748) |
| 16 | Amino acid sequence of a malonyl-CoA reductase (Mcr) (locus AY530019) |
| 17 | Amino acid sequence of a propionyl-CoA synthase (Pcs) (locus AF445079) |
| 18 | Amino acid sequence of a propionyl-CoA carboxylase alpha subunit (PccA) (locus |
| | RD1_2032) |
| 19 | Amino acid sequence of a propionyl-CoA carboxylase beta subunit (PccB) (RD1_2028) |
| 20 | Amino acid sequence of a methylmalonyl-CoA epimerase (EC 5.1.99.1) (locus CP000661) |
| 21 | Amino acid sequence of a methylmalonyl-CoA mutase (EC 5.1.99.2) (YliK) (locus |
| | NC000913.2) |
| 22 | Amino acid sequence of a succinyl-CoA:L-malate CoA transferase (SmtA) (locus |
| | DQ472736.1) |
| 23 | Amino acid sequence of a succinyl-CoA:L-malate CoA transferase (SmtB) (locus |
| | DQ472737.1) |
| 24 | Amino acid sequence of a fumarate reductase (EC 1.3.1.6) (FrdA fumarate reductase |
| | flavoprotein subunit) (AAA23437.1) |
| 25 | Amino acid sequence of a fumarate reductase (EC 1.3.1.6) (FrdB, fumarate reductase iron- |
| | sulfur subunit) (EAY46226.1) |
| 26 | Amino acid sequence of a fumarate reductase (EC 1.3.1.6) (g15 subunit) (locus |
| | NP_290787.1) |
| 27 | Amino acid sequence of a fumarate reductase (EC 1.3.1.6) (g13 subunit) (locus |
| | NP_757087.1) |
| 28 | Amino acid sequence of a fumarate hydratase (EC 4.2.1.2) (class I aerobic fumarate |
| | hydratase) (FumA) (locus CAA25204) |
| 29 | Amino acid sequence of a fumarate hydratase (EC 4.2.1.2) (class I anaerobic fumarate |
| | hydratase) (FumB) (locus AAA23827) |
| 30 | Amino acid sequence of a fumarate hydratase (EC 4.2.1.2) (class II fumarate hydratase) |
| | (FumC) (locus CAA27698) |
| 31 | Amino acid sequence of a L-malyl-CoA lyase (EC 4.2.1.2) (MclA) (locus NC_008209.1) |
| 32 | Amino acid sequence of a ATP-citrate lyase (EC. 2.3.3.8) (ATP citrate lyase subunit 1) |
| | (locus CY1089) |
| 33 | Amino acid sequence of a ATP-citrate lyase (EC. 2.3.3.8) (ATP citrate lyase subunit 2) |
| | (locus CT1088) |
| 34 | Amino acid sequence of a citryl-CoA synthetase (large subunit, CcsA) (locus BAD17844) |
| 35 | Amino acid sequence of a citryl-CoA synthetase (small subunit, CcsB) (locus BAD17846) |
| 36 | Amino acid sequence of a citryl-CoA ligase (CcI) (locus BAD17841) |
| 37 | Amino acid sequence of a malate dehydrogenase (EC 1.1.1.37) (locus CAA56810) |
| 38 | Amino acid sequence of a fumarase (also known as fumarate hydratase) (EC 4.2.1.2) |
| | (fumarase hydratase class I) (aerobic isozyme) (FumA) (JW1604) |
| 39 | Amino acid sequence of a fumarase (also known as fumarate hydratase) (EC 4.2.1.2) |
| | (fumarate hydratase class I) (anaerobic isozyme) (FumB) (JW4083) |
| 40 | Amino acid sequence of a fumarase (also known as fumarate hydratase) (EC 4.2.1.2) |
| | (fumarate hydratase class II) (FumC) (JW1603) |
| 41 | Amino acid sequence of a succinate dehydrogenase (EC 1.3.99.1) (SdhA flavoprotein |
| | subunit) (locus NP_415251) |
| 42 | Amino acid sequence of a succinate dehydrogenase (EC 1.3.99.1) (SdhB iron-sulfur |
| | subunit) (locus NP_415252) |
| 43 | Amino acid sequence of a succinate dehydrogenase (EC 1.3.99.1) (SdhC membrane anchor |
| | subunit) (locus NP_415249) |
| 44 | Amino acid sequence of a succinate dehydrogenase (EC 1.3.99.1) (SdhD membrane |
| | anchor subunit) (locus NP_415250) |
| 45 | Amino acid sequence of an acetyl-CoA:succinate CoA transferase (also known as |
| | succinyl-CoA synthetase) (EC 6.2.1.5) (succinyl-CoA synthetase subunit alpha) (SucD) |
| | (locus AAA23900) |
| 46 | Amino acid sequence of a an acetyl-CoA:succinate CoA transferase (also known as |
| | succinyl-CoA synthetase) (EC 6.2.1.5) (succinyl-CoA synthetase subunit alpha) (SucC) |
| | (locus AAA23899) |
| 47 | Amino acid sequence of a 2-oxoketoglutarate synthase (also known as alpha-ketoglutarate |
| | synthase) (EC 1.2.7.3) (KorA) (locus AB046568) |
| 48 | Amino acid sequence of a 2-oxoketoglutarate synthase (also known as alpha-ketoglutarate |
| | synthase) (EC 1.2.7.3) (KorB) (locus AB046568) |
| 49 | Amino acid sequence of a isocitrate dehydrogenase (EC 1.1.1.42) (Idh) (locus EAM42635) |
| 50 | Amino acid sequence of a NAD-dependent isocitrate dehydrogenase (EC 1.1.1.41) |
| | (Subunit 1, Idh1) (locus YNL037C) |
| 51 | Amino acid sequence of a NAD-dependent isocitrate dehydrogenase (EC 1.1.1.41) |
| | (Subunit 2, Idh2) (locus YOR136W) |
| 52 | Amino acid sequence of an aconitate hydrase 1 (AcnA) (locus b1276) |
| 53 | Amino acid sequence of an aconitate hydratase 2 (AcnB) (locus b0118) |
| 54 | Amino acid sequence of a pyruvate synthase (subunit PorA) (locus AA036986) |
| 55 | Amino acid sequence of a pyruvate synthase (subunit PorB) (locus AA036985) |
| 56 | Amino acid sequence of a pyruvate synthase (subunit PorC) (locus AA036988) |
| 57 | Amino acid sequence of a pyruvate synthase (subunit PorD) (locus AA036987) |
| 58 | Amino acid sequence of a phosphoenolpyruvate synthase (PpsA) (locus AAA24319) |
| 59 | Amino acid sequence of a phosphoenolpyruvate carboxylase (PpC) (locus CAA29332) |
| 60 | Amino acid sequence of a NADP-dependent formate dehydrogenase (EC 1.2.1.4.3) (Mt- |
| | FdhA) (locus AAB18330) |
| 61 | Amino acid sequence of a NADP-dependent formate dehydrogenase (EC 1.2.1.4.3) (beta |
| | subunit, Mt-FdhB) (locus AAB18329) |
| 62 | Amino acid sequence of a formate tetrahydrofolate ligase (EC 6.3.4.3) (locus M21507) |
| 63 | Amino acid sequence of a methenyltetrahydrofolate cyclohydrolase (also known as 5,10- |
| | methylene-tetrahydrofolate dehydrogenase) (EC 3.5.4.9 and 1.5.1.5) (locus AAA23803) |
| 64 | Amino acid sequence of a methylene tetrahydrofolate reductase (EC 1.5.1.20) (MetF) |
| | (locus CAA24747) |
| 65 | Amino acid sequence of a 5-methyltetrahydrofolate corrinoid/iron sulfur protein |
| | methyltransferase (AcsE) (locus AAA53548) |
| 66 | Amino acid sequence of a carbon monoxide dehydrogenase (subunit beta) (locus |
| | AAA23228) |
| 67 | Amino acid sequence of an acetyl-CoA synthase (subunit alpha) (locus AAA23229) |
| 68 | Amino acid sequence of a malate synthase (EC 2.3.3.9) (locus JW3974) (AceB) |
| 69 | Amino acid sequence of a malate synthase enzyme (locus JW2943) (malate synthase G) |
| | (GlcB) |
| 70 | Amino acid sequence of an isocitrate lyase (EC 4.1.3.1) (locus JW3975) (AceA) |
| 71 | Amino acid sequence of a malate dehydrogenase (EC 1.1.1.37) (locus JW3205) (Mdh) |
| 72 | Amino acid sequence of a pyruvate carboxylase (EC 6.4.4.1) (locus YGL062W) (Pyc1) |
| 73 | Amino acid sequence of a phosphoenolpyruvate carboxykinase (EC 4.1.1.49) (locus |
| | JW3366) (PckA) |
| 74 | Amino acid sequence of a fructose-1,6-bisphosphatase (EC 3.1.3.11) (locus JW4191) |
| | (Fbp) |
| 75 | Amino acid sequence of a glucose-6-phosphatase (EC 3.1.3.68) (locus YHR044C) (Dog1) |
| 76 | Amino acid sequence of a glucose-6-phosphatase (locus YHR043C) (Dog2) |
| 77 | Amino acid sequence of a pyruvate ferredoxin oxidoreductase (locus Moth_0064) |
| 78 | Amino acid sequence of a NAD⁺-dependent isocitrate dehydrogenase (EC 1.1.1.41) (locus |
| | YNL037C) (Idh1) |
| 79 | Amino acid sequence of a NAD⁺-dependent isocitrate dehydrogenase (EC 1.1.1.41) (locus |
| | YOR136W) (Idh2) |
| 80 | Amino acid sequence of a malate dehydrogenase (EC 1.1.1.37) (locus JW3205) (Mdh) |
| 81 | Amino acid sequence of a nuo operon gene (locus AF029365) (NuoA, accession |
| | AAC24985.1) |
| 82 | Amino acid sequence of a nuo operon gene (locus AF029365) (NuoB, accession |
| | AAC24986.1) |
| 83 | Amino acid sequence of a nuo operon gene (locus AF029365) (NuoC, accession |
| | AAC24987.1) |
| 84 | Amino acid sequence of a nuo operon gene (locus AF029365) (NuoD, accession |
| | AAC24988.1) |
| 85 | Amino acid sequence of a nuo operon gene (locus AF029365) (NuoE, accession |
| | AAC24989.1) |
| 86 | Amino acid sequence of a nuo operon gene (locus AF029365) (NuoF, accession |
| | AAC24991.1) |
| 87 | Amino acid sequence of a nuo operon gene (locus AF029365) (NuoG, accession |
| | AAC24995.1) |
| 88 | Amino acid sequence of a nuo operon gene (locus AF029365) (NuoH, accession |
| | AAC24997.1) |
| 89 | Amino acid sequence of a nuo operon gene (locus AF029365) (NuoI, accession |
| | AAC24999.1) |
| 90 | Amino acid sequence of a nuo operon gene (locus AF029365) (NuoJ, accession |
| | AAC25001.1) |
| 91 | Amino acid sequence of a nuo operon gene (locus AF029365) (NuoK, accession |
| | AAC25002.1) |
| 92 | Amino acid sequence of a nuo operon gene (locus AF029365) (NuoL, accession |
| | AAC25003.1) |
| 93 | Amino acid sequence of a nuo operon gene (locus AF029365) (NuoM, accession |
| | AAC25004.1) |
| 94 | Amino acid sequence of a nuo operon gene (locus AF029365) (NuoN, accession |
| | AAC25005.1) |
| 95 | Amino acid sequence of a glucose-6-phosphate dehydrogenase (EC 1.1.1.49) (Zwf) (locus |
| | JW1841) |
| 96 | Amino acid sequence of a 6-phosphogluconolactonase (EC 3.1.1.31) (Pgi) (locus JW0750) |
| 97 | Amino acid sequence of a 6-phosphogluconate dehydrogenase (EC 1.1.1.44) (Znd) (locus |
| | JW2011) |
| 98 | Amino acid sequence of a isocitrate dehydrogenase (EC 1.1.1.42) (Icd) (locus JW1122) |
| 99 | Amino acid sequence of a malic enzyme (EC 1.1.1.40) (MaeB) (locus JW2447) |
| 100 | Amino acid sequence of a pyridine nucleotide transhydrogenase (EC 1.6.1.1) (SthA or |
| | UdhA) (locus NP_418397.2) |
| 101 | Amino acid sequence of a pyridine nucleotide transhydrogenase (multisubunit of NAD(P) |
| | transhydrogenase subunit alpha) (PntA) (locus JW1595) |
| 102 | Amino acid sequence of a pyridine nucleotide transhydrogenase (NADP transhydrogenase |
| | subunit beta) (PntB) (locus JW1594) |
| 103 | Amino acid sequence of a eukaryotic light-activated proton pump (opsin) (accession |
| | AAG01180) |
| 104 | Amino acid sequence of a beta-carotene ketolase (CrtO) (locus AY705709) |
| 105 | Amino acid sequence of a succinyl-CoA synthetase subunit beta (SucC) (locus |
| | AAM71626) |
| 106 | Amino acid sequence of a succinyl-CoA synthetase, alpha subunit (SucD) (locus |
| | AAM71515) |
| 107 | Amino acid sequence of a 2-oxoglutarate synthase (EC 1.2.7.3) (locus EAM42575) |
| 108 | Amino acid sequence of a 2-oxoglutarate synthase (EC 1.2.7.3) (locus EAM42574) |
| 109 | Amino acid sequence of a 2-oxoglutarate synthase (EC 1.2.7.3) (locus EAM42853) |
| 110 | Amino acid sequence of a 2-oxoglutarate synthase (EC 1.2.7.3) (locus EAM42852) |
| 111 | Amino acid sequence of a isocitrate dehydrogenase (Icd) (EC 1.1.1.42) (locus CAE06681) |
| 112 | Amino acid sequence of a phosphoenolpyruvate synthase (PpsA) (EC 2.7.9.2) (locus |
| | AAC07865) |
| 113 | Amino acid sequence of a formyl-tetrahydrofolate synthetase (EC 6.3.4.3) (locus |
| | AAB49329) |
| 114 | Amino acid sequence of a formate-tetrahydrofolate ligase (EC 6.3.4.3) (locus BA000016) |
| 115 | Amino acid sequence of a methenyltetrahydrofolate cyclohydrolase (FolD) (EC 3.5.4.9) |
| | (locus ABC19825) |
| 116 | Amino acid sequence of a methylenetetrahydrofolate dehydrogenase (FolD) (EC 1.5.1.5 or |
| | 3.5.4.9) (locus AAO36126) |
| 117 | Amino acid sequence of a methylenetetrahydrofolate dehydrogenase (FolD) (EC 3.5.4.9) |
| | (locus BAB81529) |
| 118 | Amino acid sequence of a 5,10 methylenetetrahydrofolate reductase (MetF) (locus |
| | AAC23094) |
| 119 | Amino acid sequence of a 5,10 methylenetetrahydrofolate reductase (MetF) (locus |
| | CAA30531) |
| 120 | Amino acid sequence of a 5-methyltetrahydrofolate corrinoid/iron sulfur protein |
| | methyltransferase (AcsE) (locus ABB15216) |
| 121 | Amino acid sequence of a acetyl-CoA decarbonylase/synthase complex subunit beta |
| | (AcsB) (EC 1.2.99.2) (locus YP_360060) |
| 122 | Amino acid sequence of a beta-carotene ketolase (CrtO) with sequence homology to |
| | phytoene dehydrogenase (locus NP_293819) |
| 123 | Wild type nucleotide sequence for Proteorhodopsin 19p19 |
| 124 | Wild type nucleotide sequence for Proteorhodopsin 25f10 |
| 125 | Wild type nucleotide sequence for Proteorhodopsin BAC46A06 |
| 126 | Wild type nucleotide sequence for Proteorhodopsin BAC17h8 |
| 127 | Wild type nucleotide sequence for Candidatus Pelagibacter ubique HTCC1062 |
| | bacteriorhodopsin |
| 128 | Wild type nucleotide sequence for Salinibacter ruber DSM 13855 bacteriorhodopsin |
| 129 | Wild type nucleotide sequence for GGPP synthase crtE 25f10 |
| 130 | Wild type nucleotide sequence for GGPP synthase crtE 19p19 |
| 131 | Wild type nucleotide sequence for GGPP BAC46A06 |
| 132 | Wild type nucleotide sequence for GGPP BAC17H8 |
| 133 | Wild type nucleotide sequence for Pyrobaculum arsenaticum DSM 13514 Geranylgeranyl |
| | phosphate synthase |
| 134 | Wild type nucleotide sequence for Thermosynechococcus elongatus BP-1 geranylgeranyl |
| | pyrophosphate synthase |
| 135 | Wild type nucleotide sequence for Picrophilus torridus DSM 9790 GGPS |
| 136 | Wild type nucleotide sequence for Phytoene synthase 19p19 |
| 137 | Wild type nucleotide sequence for Phytoene synthase 25f10 |
| 138 | Wild type nucleotide sequence for Phytoene synthase BAC46A06 |
| 139 | Wild type nucleotide sequence for Phytoene syntase BAC17H8 |
| 140 | Wild type nucleotide sequence for Thermosynechococcus elongatus BP-1 Phytoene |
| | synthase |
| 141 | Wild type nucleotide sequence for Picrophilus torridus DSM 9790 Phytoene synthase |
| 142 | Wild type nucleotide sequence for Salinibacter ruber DSM 13855 phytoene synthase |
| 143 | Wild type nucleotide sequence for Phytoene dehydrogenase crtI 19p19 |
| 144 | Wild type nucleotide sequence for Phytoene dehydrogenase crtI 25F10 |
| 145 | Wild type nucleotide sequence for Phytoene dehydrogenase BAC46A06 |
| 146 | Wild type nucleotide sequence for Phytoene dehydrogenase BAC17H8 |
| 147 | Wild type nucleotide sequence for Pyrobaculum arsenaticum DSM 13514 Phytoene |
| | dehydrogenase |
| 148 | Wild type nucleotide sequence for Thermosynechococcus elongatus BP-1 Phytoene |
| | dehydrogenase |
| 149 | Wild type nucleotide sequence for Picrophilus torridus DSM 9790 Phytoene |
| | dehygrogenase |
| 150 | Wild type nucleotide sequence for Salinibacter ruber DSM 13855 Phytoene dehydrogenase |
| 151 | Wild type nucleotide sequence for Lycopene cyclase crtY 19p19 |
| 152 | Wild type nucleotide sequence for Lycopene cyclase crtY 25f10 |
| 153 | Wild type nucleotide sequence for BAC46A06 Lycopene cyclase |
| 154 | Wild type nucleotide sequence for Lycopene cyclase BAC17H8 |
| 155 | Wild type nucleotide sequence for Picrophilus torridus DSM 9790 Lycopene cyclase |
| 156 | Wild type nucleotide sequence for Carotene dehydrogenase blh 19p19 |
| 157 | Wild type nucleotide sequence for Carotene dehydrogenase blh 25f10 |
| 158 | Wild type nucleotide sequence for Carotene dehydrogenase BAC46A06 |
| 159 | Wild type nucleotide sequence for Carotene dehydrogenase BAC17H8 |
| 160 | Wild type nucleotide sequence for Picrophilus torridus DSM 9790 Carotene hydroxylase |
| 161 | Wild type nucleotide sequence for Salinibacter ruber DSM 13855 beta carotene 15 15 |
| | deoxygenase |
| 162 | Wild type nucleotide sequence for IPP delta isomerase 19p19 |
| 163 | Wild type nucleotide sequence for IPP delta isomerase 25f10 |
| 164 | Wild type nucleotide sequence for IPP isomerase BAC46A06 |
| 165 | Wild type nucleotide sequence for IPP delta isomerase BAC17H8 |
| 166 | Wild type nucleotide sequence for Picrophilus torridus DSM 9790 IPP |
| 167 | Wild type nucleotide sequence for IPP Delta Isomerase Pyrobaculum arsenaticum DSM |
| | 13514 |
| 168 | Wild type nucleotide sequence for Salinibacter ruber DSM 13855 IPP |
| 169 | Optimized amino acid sequence for Salinibacter ruber DSM 13855 IPP |
| 170 | Optimized nucleotide sequence for Salinibacter ruber DSM 13855 IPP |
| 171 | Optimized amino acid sequence for IPP Delta Isomerase Pyrobaculum arsenaticum DSM |
| | 13514 |
| 172 | Optimized nucleotide sequence for IPP Delta Isomerase Pyrobaculum arsenaticum DSM |
| | 13514 |
| 173 | Optimized amino acid sequence for Picrophilus torridus DSM 9790 IPP |
| 174 | Optimized nucleotide sequence for Picrophilus torridus DSM 9790 IPP |
| 175 | Optimized amino acid sequence for IPP delta isomerase BAC17H8 |
| 176 | Optimized nucleotide sequence for IPP delta isomerase BAC17H8 |
| 177 | Optimized amino acid sequence for IPP isomerase BAC46A06 |
| 178 | Optimized nucleotide sequence for IPP isomerase BAC46A06 |
| 179 | Optimized amino acid sequence for IPP delta isomerase 25f10 |
| 180 | Optimized nucleotide sequence for IPP delta isomerase 25f10 |
| 181 | Optimized amino acid sequence for IPP delta isomerase 19p19 |
| 182 | Optimized nucleotide sequence for IPP delta isomerase 19p19 |
| 183 | Optimized amino acid sequence for Salinibacter ruber DSM 13855 beta carotene 15 15 |
| | deoxygenase |
| 184 | Optimized nucleotide sequence for Salinibacter ruber DSM 13855 beta carotene 15 15 |
| | deoxygenase |
| 185 | Optimized amino acid sequence for Picrophilus torridus DSM 9790 Carotene hydroxylase |
| 186 | Optimized nucleotide sequence for Picrophilus torridus DSM 9790 Carotene hydroxylase |
| 187 | Optimized amino acid sequence for Carotene dehydrogenase BAC17H8 |
| 188 | Optimized nucleotide sequence for Carotene dehydrogenase BAC17H8 |
| 189 | Optimized amino acid sequence for Carotene dehydrogenase BAC46A06 |
| 190 | Optimized nucleotide sequence for Carotene dehydrogenase BAC46A06 |
| 191 | Optimized amino acid sequence for Carotene dehydrogenase blh 25f10 |
| 192 | Optimized nucleotide sequence for Carotene dehydrogenase blh 25f10 |
| 193 | Optimized amino acid sequence for Carotene dehydrogenase blh 19p19 |
| 194 | Optimized nucleotide sequence for Carotene dehydrogenase blh 19p19 |
| 195 | Optimized amino acid sequence for Picrophilus torridus DSM 9790 Lycopene cyclase |
| 196 | Optimized nucleotide sequence for Picrophilus torridus DSM 9790 Lycopene cyclase |
| 197 | Optimized amino acid sequence for Lycopene cyclase BAC17H8 |
| 198 | Optimized nucleotide sequence for Lycopene cyclase BAC17H8 |
| 199 | Optimized amino acid sequence for BAC46A06 Lycopene cyclase |
| 200 | Optimized nucleotide sequence for BAC46A06 Lycopene cyclase |
| 201 | Optimized amino acid sequence for Lycopene cyclase crtY 25f10 |
| 202 | Optimized nucleotide sequence for Lycopene cyclase crtY 25f10 |
| 203 | Optimized amino acid sequence for Lycopene cyclase crtY 19p19 |
| 204 | Optimized nucleotide sequence for Lycopene cyclase crtY 19p19 |
| 205 | Optimized amino acid sequence for Salinibacter ruber DSM 13855 Phytoene |
| | dehydrogenase |
| 206 | Optimized nucleotide sequence for Salinibacter ruber DSM 13855 Phytoene |
| | dehydrogenase |
| 207 | Optimized amino acid sequence for Picrophilus torridus DSM 9790 Phytoene |
| | dehygrogenase |
| 208 | Optimized nucleotide sequence for Picrophilus torridus DSM 9790 Phytoene |
| | dehygrogenase |
| 209 | Optimized amino acid sequence for Thermosynechococcus elongatus BP-1 Phytoene |
| | dehydrogenase |
| 210 | Optimized nucleotide sequence for Thermosynechococcus elongatus BP-1 Phytoene |
| | dehydrogenase |
| 211 | Optimized amino acid sequence for Pyrobaculum arsenaticum DSM 13514 Phytoene |
| | dehydrogenase |
| 212 | Optimized nucleotide sequence for Pyrobaculum arsenaticum DSM 13514 Phytoene |
| | dehydrogenase |
| 213 | Optimized amino acid sequence for Phytoene dehydrogenase BAC17H8 |
| 214 | Optimized nucleotide sequence for Phytoene dehydrogenase BAC17H8 |
| 215 | Optimized amino acid sequence for Phytoene dehydrogenase BAC46A06 |
| 216 | Optimized nucleotide sequence for Phytoene dehydrogenase BAC46A06 |
| 217 | Optimized amino acid sequence for Phytoene dehydrogenase crtI 25F10 |
| 218 | Optimized nucleotide sequence for Phytoene dehydrogenase crtI 25F10 |
| 219 | Optimized amino acid sequence for Phytoene dehydrogenase crtI 19p19 |
| 220 | Optimized nucleotide sequence for Phytoene dehydrogenase crtI 19p19 |
| 221 | Optimized amino acid sequence for Salinibacter ruber DSM 13855 phytoene synthase |
| 222 | Optimized nucleotide sequence for Salinibacter ruber DSM 13855 phytoene synthase |
| 223 | Optimized amino acid sequence for Picrophilus torridus DSM 9790 Phytoene synthase |
| 224 | Optimized nucleotide sequence for Picrophilus torridus DSM 9790 Phytoene synthase |
| 225 | Optimized amino acid sequence for Thermosynechococcus elongatus BP-1 Phytoene |
| | synthase |
| 226 | Optimized nucleotide sequence for Thermosynechococcus elongatus BP-1 Phytoene |
| | synthase |
| 227 | Optimized amino acid sequence for Phytoene syntase BAC17H8 |
| 228 | Optimized nucleotide sequence for Phytoene syntase BAC17H8 |
| 229 | Optimized amino acid sequence for Phytoene synthase BAC46A06 |
| 230 | Optimized nucleotide sequence for Phytoene synthase BAC46A06 |
| 231 | Optimized amino acid sequence for Phytoene synthase 25f10 |
| 232 | Optimized nucleotide sequence for Phytoene synthase 25f10 |
| 233 | Optimized amino acid sequence for Phytoene synthase 19p19 |
| 234 | Optimized nucleotide sequence for Phytoene synthase 19p19 |
| 235 | Optimized amino acid sequence for Picrophilus torridus DSM 9790 GGPS |
| 236 | Optimized nucleotide sequence for Picrophilus torridus DSM 9790 GGPS |
| 237 | Optimized amino acid sequence for Thermosynechococcus elongatus BP-1 GGPS |
| 238 | Optimized nucleotide sequence for Thermosynechococcus elongatus BP-1 GGPS |
| 239 | Optimized amino acid sequence for Pyrobaculum arsenaticum DSM 13514 GGPS |
| 240 | Optimized nucleotide sequence for Pyrobaculum arsenaticum DSM 13514 GGPS |
| 241 | Optimized amino acid sequence for GGPP BAC17H8 |
| 242 | Optimized nucleotide sequence for GGPP BAC17H8 |
| 243 | Optimized amino acid sequence for GGPP BAC46A06 |
| 244 | Optimized nucleotide sequence for GGPP BAC46A06 |
| 245 | Optimized amino acid sequence for GGPP synthase crtE 19p19 |
| 246 | Optimized nucleotide sequence for GGPP synthase crtE 19p19 |
| 247 | Optimized amino acid sequence for GGPP synthase crtE 25f10 |
| 248 | Optimized nucleotide sequence for GGPP synthase crtE 25f10 |
| 249 | Optimized amino acid sequence for Salinibacter ruber DSM 13855 bacteriorhodopsin |
| 250 | Optimized nucleotide sequence for Salinibacter ruber DSM 13855 bacteriorhodopsin |
| 251 | Optimized amino acid sequence for Candidatus Pelagibacter ubique HTCC1062 |
| | bacteriorhodopsin |
| 252 | Optimized nucleotide sequence for Candidatus Pelagibacter ubique HTCC1062 |
| | bacteriorhodopsin |
| 253 | Optimized amino acid sequence for Proteorhodopsin BAC17h8 |
| 254 | Optimized nucleotide sequence for Proteorhodopsin BAC17h8 |
| 255 | Optimized amino acid sequence for Proteorhodopsin BAC46A06 |
| 256 | Optimized nucleotide sequence for Proteorhodopsin BAC46A06 |
| 257 | Optimized amino acid sequence for Proteorhodopsin 25f10 |
| 258 | Optimized nucleotide sequence for Proteorhodopsin 25f10 |
| 259 | Optimized amino acid sequence for Proteorhodopsin 19p19 |
| 260 | Optimized nucleotide sequence for Proteorhodopsin 19p19 |
| 261 | Optimized amino acid sequence for Salinibacter ruber DSM 13855 fructose-bisphosphate |
| | aldolase |
| 262 | Optimized nucleotide sequence for Salinibacter ruber DSM 13855 fructose-bisphosphate |
| | aldolase |
| 263 | Wild type nucleotide sequence for Salinibacter ruber DSM 13855 fructose-bisphosphate |
| | aldolase |
| 264 | Optimized amino acid sequence for Synechococcus sp. PCC 7002 fructose-bisphosphate |
| | aldolase, class I |
| 265 | Optimized nucleotide sequence for Synechococcus sp. PCC 7002 fructose-bisphosphate |
| | aldolase, class I |
| 266 | Wild type nucleotide sequence for Synechococcus sp. PCC 7002 fructose-bisphosphate |
| | aldolase, class I |
| 267 | Optimized nucleotide sequence for Synechococcus elongatus PCC 7942 sedoheptulose- |
| | 1,7-bisphosphatase |
| 268 | Wild type nucleotide sequence for Synechococcus elongatus PCC 7942 sedoheptulose-1,7- |
| | bisphosphatase |
| 269 | Optimized nucleotide sequence for Thermosynechococcus elongatus BP-1 sedoheptulose- |
| | 1,7-bisphosphatase |
| 270 | Wild type nucleotide sequence for Thermosynechococcus elongatus BP-1 sedoheptulose- |
| | 1,7-bisphosphatase |
| 271 | Optimized nucleotide sequence for phosphoribulokinase gene prkA from Synechococcus |
| | sp. PCC7942 (Genbank: AB035257) |
| 272 | Wild type nucleotide sequence rbcL gene (enzyme ribulose-bisphosphate-carboxylase, EC |
| | 4.1.1.39) from Synechococcus PCC6301 |
| 273 | Wild type amino acid sequence rbcL gene (enzyme ribulose-bisphosphate-carboxylase, EC |
| | 4.1.1.39) from Synechococcus PCC6301 |
| 274 | Optimized nucleotide sequence for the rbcL gene |
| 275 | Wild type nucleotide sequence Synechococcus PCC6301 for the rbcS gene (enzyme |
| | ribulose-bisphosphate-carboxylase, EC 4.1.1.39) |
| 276 | Wild type amino acid sequence Synechococcus PCC6301 for the rbcS gene (enzyme |
| | ribulose-bisphosphate-carboxylase, EC 4.1.1.39) |
| 277 | Optimized nucleotide sequence for the rbcS gene |

All references to publications, including scientific publications, treatises, pre-grant patent publications, and issued patents are hereby incorporated by reference in their entirety for all purposes. The teachings of the specification are intended to exemplify but not limit the invention, the scope of which is determined by the following claims.

What is claimed is:

1. An engineered cell comprising at least two engineered nucleic acids, wherein at least one engineered nucleic acid is selected from a group consisting of a light capture nucleic acid, a carbon dioxide fixation pathway nucleic acid, a NADH pathway nucleic acid, and a NADPH pathway nucleic acid; and wherein a second engineered nucleic acid is selected from a distinct member of said group.

2. The cell of claim 1, wherein said cell is light dependent or fixes carbon.

3. The cell of claim 1, wherein said cell has engineered phototrophic activity.

4. The cell of claim 1, wherein said cell is synthetophototrophic.

5. The cell of claim 1, wherein said cell fixes carbon and is synthetophototrophic.

6. The cell of claim 1, wherein said cell is photoautotrophic in the presence of light and heterotrophic in the absence of light.

7. The cell of claim 1, wherein said cell is a microorganism selected from the group consisting of Acetobacter aceti, Bacillus subtilis, Clostridium ljungdahlii, Clostridium thermocellum, Escherichia coli, Penicillium chrysogenum, Pichia pastoris, Saccharomyces cerevisiae, Schizosaccharomyces pombe, Pseudomonas fluorescens and Zymomonas mobilis.

8. The cell of claim 7, wherein said cell is an Escherichia coli cell.

9. The cell of claim 1, wherein said at least one engineered nucleic acid is an exogenous nucleic acid.

10. The cell of claim 1, wherein said at least one engineered nucleic acid is a modified endogenous gene.

11. The cell of claim 1, further comprising an additional modified endogenous gene.

12. The cell of claim 1, wherein said engineered nucleic acids are selected from at least three members of the group consisting of a light capture nucleic acid, a carbon dioxide fixation pathway nucleic acid, a NADH pathway nucleic acid, and a NADPH pathway nucleic acid.

13. The cell of claim 1, wherein said cell comprises at least one engineered light capture nucleic acid, at least one engineered carbon dioxide fixation pathway nucleic acid, at least one engineered NADH pathway nucleic acid, and at least one engineered NADPH pathway nucleic acid.

14. The cell of claim 1, wherein said cell comprises at least one engineered light capture nucleic acid and at least one engineered carbon dioxide fixation pathway nucleic acid.

15. The cell of claim 1, wherein at least one engineered nucleic acid is a light capture nucleic acid selected from the group consisting of proteorhodopsin, bacteriorhodopsin, deltarhodopsin, xanthorhodopsin, Leptosphaeria maculans opsin, isopentenyl-diphosphate delta-isomerase, 15,15′-beta-carotene dioxygenase, lycopene cyclase, phytoene synthase, phytoene dehydrogenase, geranylgeranyl pyrophosphate synthetase, beta-carotene ketolase, photosystem P840 reaction center large subunit, pscA, photosystem P840 reaction center iron-sulfur protein, pscB, photosystem P840 reaction center cytochrome c-551, pscC, photosystem P840 reaction center protein, pscD, bacteriochlorophyl a binding protein, Fenna-Mathews-Olson protein, FMO, Photosystem I P700 chlorophyll A apoproptein A1, psaA, Photosystem I P700 chlorophyll A apoproptein A2, psaB, Photosystem I iron-sulfur center subunit VII, psaC, Photosystem I reaction center subunit II, psaD, Photosystem I reaction centre subunit IV PsaE, Photosystem I reaction centre subunit IX PsaJ, Photosystem I reaction centre subunit III precursor (PSI-F), Photosystem I reaction centre subunit XII PsaM, Photosystem I reaction center subunit PsaK, Photosystem I assembly protein, Photosystem I subunit VIII PsaI, Photosystem I reaction centre subunit XI PsaL, Photosystem II protein X PsbX, Photosystem II reaction center D1, Photosystem II manganese-stabilizing protein PsbO, Photosystem II 10 kDa phosphoprotein PsbH, Photosystem II reaction center N protein PsbN, Photosystem II protein PsbI, Photosystem II protein PsbK, Photosystem II stability/assembly factor, Cytochrome b559 alpha subunit PsbE, Cytochrome b559 beta chain PsbF, Photosystem II protein L PsbL, Photosystem II protein J PsbJ, PucC protein, Photosystem II reaction center T PsbT, Photosystem II chlorophyll a-binding protein CP47 homolog, Photosystem II protein M PsbM, Photosystem II protein Psb27, Photosystem II protein Y PsbY, Photosystem II reaction centre W protein, Photosystem TI protein P PsbP, Flavodoxin, IsiB, Photosystem II reaction center D2, Photosystem II chlorophyll a-binding protein CP43 homolog, and a Homolog of PsbF protein.

16. The cell of claim 15, wherein at least one engineered nucleic acid is proteorhodopsin.

17. The cell of claim 15 or 16, wherein said cell generates proton motive force, and wherein said proton motive force promotes the growth of said cell in a light-dependent manner.

18. The cell of claim 17, wherein the growth of said cell is in the presence of salt.

19. The cell of claim 17, wherein said proton motive force is generated by proteorhodopsin.

20. The cell of claim 16, further comprising engineered rbcL nucleic acid, engineered rbcS nucleic acid, and engineered phosphoribulokinase.

21. The cell of claim 1, wherein at least one engineered nucleic acid is a carbon dioxide fixation pathway nucleic acid selected from the group consisting of a functional hydroxyproprionate cycle nucleic acid, a reductive TCA cycle nucleic acid, a reductive acetyl coenzyme A pathway nucleic acid, a reductive pentose phosphate cycle nucleic acid, a glyoxylate shunt pathway nucleic acid, a Calvin cycle nucleic acid and a gluconeogenesis pathway nucleic acid.

22. The cell of claim 21, wherein at least one engineered nucleic acid is a carbon dioxide fixation pathway nucleic acid selected from the group consisting of acetyl-CoA carboxylase (subunit alpha), acetyl-CoA carboxylase (subunit beta), biotin-carboxyl carrier protein (accB), biotin-carboxylase, malonyl-CoA reductase, 3-hydroxypropionyl-CoA synthase, propionyl-CoA carboxylase (subunit alpha), propionyl-CoA carboxylase (subunit beta), methylmalonyl-CoA epimerase, methylmalonyl-CoA mutase, succinyl-CoA:L-malate CoA transferase (subunit alpha), succinyl-CoA:L-malate CoA transferase (subunit beta), fumarate reductase—frdA-flavoprotein subunit, fumarate reductase iron-sulfur subunit-frdb, g15 subunit [fumarate reductase subunit c], g13 subunit [fumarate reductase subunit D], fumarate hydratase—class I aerobic (fumA), L-malyl-CoA lyase, ATP-citrate lyase, subunit 1, ATP-citrate lyase, subunit 2, citryl-CoA synthase (large subunit, citryl-CoA synthase (small subunit), citryl-CoA ligase, malate dehydrogenase, fumarase hydratase (aerobic isozyme, fumA), succinate dehydrogenase (flavoprotein subunit—SdhA), SdhB iron-sulfur subunit, SdhC membrane anchor subunit, SdhD membrane anchor subunit, succinyl-CoA synthetase subunit alpha (sucD), succinyl-CoA synthetase subunit beta (sucC), alpha-ketoglutarate subunit alpha-korA, alpha-ketoglutarate subunit beta-korB, isocitrate dehydrogenase—NADP dependent, isocitrate dehydrogenase—NAD dependent Subunit 1, isocitrate dehydrogenase—NAD depend. Subunit 2, aconitate hydratase 1 (acnA), aconitate hydratase 2 (acnB), pyruvate synthase, subunit A porA, pyruvate synthase, subunit B porB, pyruvate synthase, subunit C porC, pyruvate synthase, subunit D porD, phosphoenolpyruvate synthase—ppsA, PEP carboxylase, ppC, NADP-dependent formate dehydrogenase—subunit A Mt-fdhA, NADP-dependent formate dehydrogenase—subunit B Mt-fdhB, formate tetrahydrofolate ligase, methenyltetrahydrofolate cyclohydrolase, methylene tetrahydrofolate reductase, metF, 5-methyltetrahydrofolate corrinoid/iron sulfur protein methyltransferase, acsE, carbon monoxide dehydrogenase/acetyl-CoA synthase—subunit alpha, carbon monoxide dehydrogenase/acetyl-CoA synthase—subunit beta, malate synthase—aceB, isocitrate lyase—aceA, malate dehydrogenase, pyruvate carboxylase, phosphoenolpyruvate carboxykinase, fructose-1,6-bisphosphatase, glucose-6-phosphatase—dog1, pyruvate ferredoxin:oxidoreductase with pyruvate synthase activity, fructose-1,6-bisphosphatase (FBPase) and sedoheptulose-1,7-bisphosphatase (SBPase), bifunctional, cbbF, glyceraldehyde-3-phosphate dehydrogenase (GAPDH), cbbG, phosphoribulokinase (PRK), cbbP, CP12, transketolase, cbbT, fructose 1,6-bisphosphate aldolase, cbbA, pentose-5-phosphate-3-epimerase, cbbE, ribose 5-phosphate isomerase, phosphoglycerate kinase, triosephosphate isomerase, tpiA, Ribulose-1,5-bisphosphate carbyxlase/oxygenase (RubisCo)-small subunit—cbbS, Ribulose-1,5-bisphosphate carbyxlase/oxygenase (RubisCo)-large subunit cbbL, Rubisco activase, rbcL, rbcS, Salinibacter fructose-bisphosphate aldolase, Synechococcus sp. 7002 fructose-bisphosphate aldolase (class I), Synechococcus elongatus PCC 7942 sedoheptulose-1,7-bisphosphatase, and T. elongatus BP-1 sedoheptulose-1,7-bisphosphatase.

23. The cell of claim 22 wherein at least one engineered nucleic acid is a codon-optimized carbon dioxide fixation pathway nucleic acid selected from the group consisting of Salinibacter fructose-bisphosphate aldolase, Synechococcus sp. 7002 fructose-bisphosphate aldolase (class I), Synechococcus elongatus PCC 7942 sedoheptulose-1,7-bisphosphatase, and T. elongatus BP-1 sedoheptulose-1,7-bisphosphatase.

24. The cell of claim 22 or 23, wherein said cell generates proton motive force, and wherein said proton motive force promotes the growth of said cell in a light-dependent manner.

25. The cell of claim 24, wherein said growth is in the presence of salt.

26. The cell of claim 24, wherein said proton motive force is generated by proteorhodopsin.

27. The cell of claim 26, wherein said cell comprises engineered rbcL nucleic acid, engineered rbcS nucleic acid, and engineered phosphoribulokinase.

28. The cell of claim 22, wherein said carbon dioxide fixation pathway nucleic acid is a Woods-Ljungdahl pathway nucleic acid.

29. The cell of claim 27, further comprising an engineered glyoxylate shunt pathway nucleic acid and an exogenous gluconeogenesis pathway nucleic acid.

30. The cell of claim 1, wherein at least one engineered nucleic acid is a NADH pathway nucleic acid selected from the group consisting of soluble pyridine nucleotide transhydrogenase—udhA, membrane-bound pyridine nucleotide transhydrogenase—pntAB, NAD+-dependent isocitrate dehydrogenase—idh, NAD+-dependent isocitrate dehydrogenase—idh2, malate dehydrogenase, and NADH:ubiquinone oxidoreductase—OPERON (a-n).

31. The cell of claim 1, wherein at least one engineered nucleic acid is an endogenous NADH pathway nucleic acid selected from the group consisting of a nuo gene, a ndh gene, cytochrome bo, and cytochrome bd.

32. The cell of claim 31, wherein said endogenous NADH pathway nucleic acid comprises a deletion or modification that disrupts said pathway.

33. The cell of claim 30, comprising at least two engineered NADH pathway nucleic acids, wherein said at least two engineered NADH pathway nucleic acids include a soluble pyridine nucleotide dehydrogenase and a NAD⁺-dependent iso citrate dehydrogenase.

34. The cell of claim 1, wherein at least one engineered nucleic acid is a NADPH pathway nucleic acid selected from the group consisting of glucose-6-phosphate dehydrogenase, zwf, 6-phosphogluconolactonase -pgi, 6-phosphogluconate dehydrogenase, gnd, NADP-dependent isocitrate dehydrogenase, NADP-dependent malic enzyme, soluble pyridine nucleotide transhydrogenase—udhA, or membrane-bound pyridine nucleotide transhydrogenase, subunit alpha, pntA and subunit beta, pntB.

35. The cell of claim 34, comprising at least two engineered NADPH pathway nucleic acids, wherein said at least two NADPH pathway nucleic acids include a soluble nucleotide dehydrogenase and a glucose-6-phosphate dehydrogenase.

36. The cell of claim 1, wherein one or more acetyl-CoA flux nucleic acids are expressed or inhibited.

37. A host cell generating proton motive force, wherein said proton motive force promotes the light-dependent growth of said cell.

38. The host cell of claim 37, wherein the growth of said cell is in the presence of salt.

39. The cell of claim 38, wherein said salt concentration is about 0.3M.

40. A host cell, wherein said host cell is engineered to capture light and fix carbon dioxide.

41. A method for producing carbon products, wherein said products comprise biological sugars, hydrocarbon products, solid forms of carbon, fuels, biofuels or pharmaceutical agents, comprising culturing the cell of any of claims 1, 37 or 40 under conditions sufficient to promote the generation of said carbon products; and collecting or separating the carbon product produced by said cell.

42. The method of claim 41, wherein said cell is cultivated in a bioreactor supplied with a concentrated carbon dioxide source.

43. The method of claim 42, wherein said concentrated carbon dioxide source is offgas from one or more sources selected from the group consisting of a coal plant, refinery, cement production facility, brewery, or natural gas facility.
